(19) United States
(12) Patent Application Publication
(10) Pub. No.: US 2018/0325085 A1
(43) Pub. Date: Nov. 15, 2018

(54) COMPOSITIONS AND METHODS RELATING TO TUMOR ANALYSIS

(71) Applicant: The Jackson Laboratory, Bar Harbor, ME (US)

(72) Inventors: Michael V. Wiles, Mount Desert, ME (US); Vishnu Hosur, Bar Harbor, ME (US); Leonard D. Shultz, Bar Harbor, ME (US)

(21) Appl. No.: 15/771,219

(22) PCT Filed: Oct. 31, 2016
(86) PCT No.: PCT/US2016/059711

§ 371 Date: Apr. 27, 2018

Related U.S. Application Data
(60) Provisional application No. 62/248,417, filed on Oct. 30, 2015.

(51) Int. Cl. A01K 67/027; A61K 49/00

(52) U.S. Cl. CPC A01K 67/0276; G01N 33/502; A61K 49/0008

(57) ABSTRACT

A genetically modified NOD.Cg-Prkdc^{scid} Il2rg^{tm1Wjl}/SzJ mouse is provided by the present invention wherein the genome of the mouse includes a mutated Rhbdf2 gene such that the mouse expresses a mutant iRhom2 protein, wherein the mutant iRhom2 protein differs from wild-type iRhom2 protein due to one or more mutations selected from p.I156T, p.D158N and p.P159L, and wherein the mouse is characterized by hairless phenotype and increased growth of an exogenous tumor compared to a mouse of the same genetic background which express wild-type iRhom2 protein.

### REFERENCE TO RELATED APPLICATION

This application claims priority from U.S. Provisional Patent Application Ser. No. 62/248,417, filed Oct. 30, 2015, the entire content of which is incorporated herein by reference.

### FIELD OF THE INVENTION

According to specific aspects, genetically modified NOD.Cg-Prkdc^{scid} Il2rg^{tm1Wjl}/SzJ (NSG) mice are provided by the present invention wherein the genome of the mice includes a mutated Rhbdf2 gene such that the mice express a mutant iRhom2 protein, wherein the mutant iRhom2 protein differs from wild-type iRhom2 protein due to one or more mutations selected from p.I156T, p.D158N and p.P159L, and wherein the mice are characterized by hairless phenotype and increased growth of an exogenous tumor compared to mice of the same genetic background which express wild-type iRhom2 protein.

### BACKGROUND OF THE INVENTION

While significant advances have been made in diagnosis and treatment of cancer in recent years, the disease continues to be common and widespread. The US National Cancer Institute's Surveillance Epidemiology and End Results (SEER) Database indicates that 1 in 2 men and 1 in 3 women in the United States are considered at risk of developing some type of cancer based on incidence and mortality data for the United States from 2010 through 2012. The same database indicates that 1 in 4 men and 1 in 5 women in the United States are considered at risk of dying due to some type of cancer.

Advances in diagnosis and treatment of cancer are required to improve chances of survival. However, most cancer models used for study and evaluation cancer and new drugs consist of cell lines in vitro and analyses of such in vitro models can be of limited value given the complexity of in vivo physiological processes. Current in vivo cancer models are frequently limited in application, particularly where analysis is desirable as soon as possible, such as analysis of patient-derived tumor cells in xenograft tumors grown in vivo.

Thus, there is a continuing need for in vivo cancer models and methods for identifying an anti-tumor compositions and treatments using the in vivo cancer models.

### SUMMARY OF THE INVENTION

A genetically modified NSG mouse is provided according to aspects of the present invention wherein the genome of the mouse includes a mutated Rhbdf2 gene such that the mouse expresses mutant iRhom2 protein which differs from wild-type mouse iRhom2 protein due to one or more mutations selected from: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein.

A genetically modified NSG mouse is provided according to aspects of the present invention wherein the genome of the mouse includes a mutated Rhbdf2 gene such that the mouse expresses mutant iRhom2 protein which differs from wild-type mouse iRhom2 protein due to one or more mutations selected from: p.I156T, p.D158N and p.P159L, wherein the genetically modified NSG mouse is characterized by hairless phenotype and increased growth of a xenogeneic tumor compared to a mouse of the same genetic background which express wild-type iRhom2 protein, and wherein the genetically modified NSG mouse includes a xenogeneic tumor cell.

A genetically modified NSG mouse is provided according to aspects of the present invention wherein the genome of the mouse includes a mutated Rhbdf2 gene such that the mouse expresses mutant iRhom2 protein which differs from wild-type mouse iRhom2 protein due to one or more mutations selected from: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein, and wherein the genetically modified NSG mouse includes a xenogeneic tumor cell obtained from a tumor of a human subject.

A genetically modified NOD.Cg-Prkdc^{scid}Il2rg^{tm1Wjl}Rhbdf^{P159L}/SzJ mouse is provided according to aspects of the present invention. A genetically modified NOD.Cg-Prkdc^{scid}Il2rg^{tm1Wjl}Rhbdf^{P159L}/SzJ mouse is provided according to aspects of the present invention which includes a xenogeneic tumor cell. A genetically modified NOD.Cg-Prkdc^{scid}Il2rg^{tm1Wjl}Rhbdf^{P159L}/SzJ mouse is provided according to aspects of the present invention which includes a xenogeneic tumor cell obtained from a tumor of a human subject.

According to aspects, the present invention provides a method for producing a mouse model system for assessment of a xenogeneic tumor cell, which includes providing a genetically modified NSG mouse, wherein the genome of the mouse includes a mutated Rhbdf2 gene such that the mouse expresses mutant iRhom2 protein which differs from wild-type mouse iRhom2 protein due to one or more mutations in the N-terminal region of iRhom2 selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein; providing a xenogeneic tumor cell; and administering the xenogeneic tumor cell to the genetically modified NSG mouse, thereby producing a mouse model system for assessment of a xenogeneic tumor cell.

According to aspects, the present invention provides a method for producing a mouse model system for assessment of a xenogeneic tumor cell obtained from a tumor of a human subject which includes providing a genetically modified NSG mouse, wherein the genome of the mouse includes a mutated Rhbdf2 gene such that the mouse expresses mutant iRhom2 protein which differs from wild-type mouse iRhom2 protein due to one or more mutations in the N-terminal region of iRhom2 selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein; providing a xenogeneic tumor cell obtained from a tumor of a human subject; and administering the xenogeneic tumor cell to the genetically modified NSG mouse, thereby producing a mouse model system for assessment of a xenogeneic tumor cell obtained from a tumor of a human subject.

According to aspects, the present invention provides a method for producing a mouse model system for assessment of a xenogeneic tumor cell which includes providing a NOD.Cg-Prkdc^{scid}Il2rg^{tm1Wjl}Rhbdf^{P159L}/SzJ mouse, wherein the NOD.Cg-Prkdc^{scid}Il2rg^{tm1Wjl}Rhbdf^{P159L}/SzJ mouse is characterized by hairless phenotype and increased growth of a xenogeneic tumor compared to a mouse of the same genetic background which expresses wild-type iRhom2 protein; providing a xenogeneic tumor cell; and administering the xenogeneic tumor cell to the NOD.Cg-Prkdc^{scid}Il2rg^{tm1Wjl}Rhbdf^{P159L}/SzJ mouse, thereby producing a mouse model system for assessment of a xenogeneic tumor cells.

According to aspects, the present invention provides a method for producing a mouse model system for assessment of a xenogeneic tumor cell which includes providing a NOD.Cg-Prkdc^{scid}Il2rg^{tm1Wjl}Rhbdf^{P159L}/SzJ mouse, wherein the NOD.Cg-Prkdc^{scid}Il2rg^{tm1Wjl}Rhbdf^{P159L}/SzJ mouse is characterized by hairless phenotype and increased growth of a xenogeneic tumor compared to a mouse of the same genetic background which expresses wild-type iRhom2 protein; providing a xenogeneic tumor cell obtained from a tumor of a human subject; and administering the xenogeneic tumor cell to the NOD.Cg-Prkdc^{scid}Il2rg^{tm1Wjl}Rhbdf^{P159L}/SzJ mouse, thereby producing a mouse model system for assessment of a xenogeneic tumor cell.

According to aspects, the present invention provides a method for identifying an anti-tumor composition which includes providing a genetically modified NSG mouse, wherein the genome of the mouse includes a mutated Rhbdf2 gene such that the mouse expresses mutant iRhom2 protein which differs from wild-type mouse iRhom2 protein due to one or more mutations selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein; providing a xenogeneic tumor cell; administering the xenogeneic tumor cell to the genetically modified NSG mouse, producing a genetically modified NSG mouse including xenogeneic tumor cells; administering a test substance to the genetically modified NSG mouse including xenogeneic tumor cells; assaying a response of a xenogeneic tumor cell to the test substance following administration of the test substance to the genetically modified NSG mouse including a xenogeneic tumor cell; and comparing the response to a standard to determine the effect of the test substance on the xenogeneic tumor cells, wherein an inhibitory effect of the test substance on the xenogeneic tumor cell identifies the test substance as an anti-tumor composition.

According to aspects, the present invention provides a method for identifying an anti-tumor composition which includes providing a genetically modified NSG mouse, wherein the genome of the mouse includes a mutated Rhbdf2 gene such that the mouse expresses mutant iRhom2 protein which differs from wild-type mouse iRhom2 protein due to one or more mutations in the N-terminal region of iRhom2 selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein; providing a xenogeneic tumor cell obtained from a tumor of a human subject; administering the xenogeneic tumor cell obtained from a tumor of a human subject to the genetically modified NSG mouse, producing a genetically modified NSG mouse including a xenogeneic tumor cell obtained from a tumor of a human subject; administering a test substance to the genetically modified NSG mouse having the administered xenogeneic tumor cell obtained from a tumor of a human subject; assaying a response of a xenogeneic tumor cell obtained from a tumor of a human subject in the mouse to the test substance following administration of the test substance to the genetically modified NSG mouse including a xenogeneic tumor cell obtained from a tumor of a human subject; and comparing the response to a standard to determine the effect of the test substance on a xenogeneic tumor cell obtained from a tumor of a human subject, wherein an inhibitory effect of the test substance on a xenogeneic tumor cell obtained from a tumor of a human subject identifies the test substance as an anti-tumor composition.

According to aspects, the present invention provides a method for identifying an anti-tumor composition which includes providing a NOD.Cg-Prkdc^{scid}Il2rg^{tm1Wjl}Rhbdf^{P159L}/SzJ mouse, wherein the NOD.Cg-Prkdc^{scid}Il2rg^{tm1Wjl}Rhbdf^{P159L}/SzJ mouse is characterized by hairless phenotype and increased growth of a xenogeneic tumor compared to a mouse of the same genetic background which expresses wild-type iRhom2 protein; providing a xenogeneic tumor cell; administering the xenogeneic tumor cell to the NOD.Cg-Prkdc^{scid}Il2rg^{tm1Wjl}Rhbdf^{P159L}/SzJ mouse, producing a NOD.Cg-Prkdc^{scid}Il2rg^{tm1Wjl}Rhbdf^{P159L}/SzJ mouse including a xenogeneic tumor cell; administering a test substance to the NOD.Cg-Prkdc^{scid}Il2rg^{tm1Wjl}Rhbdf^{P159L}/SzJ mouse including a xenogeneic tumor cell; assaying a response of a xenogeneic tumor cell to the test substance following administration of the test substance to the NOD.Cg-Prkdc^{scid}Il2rg^{tm1Wjl}Rhbdf^{P159L}/SzJ mouse including a xenogeneic tumor cell; and comparing the response to a standard to determine the effect of the test substance on a xenogeneic tumor cell, wherein an inhibitory effect of the test substance on a xenogeneic tumor cell identifies the test substance as an anti-tumor composition.

According to aspects, the present invention provides a method for identifying an anti-tumor composition which includes providing a NOD.Cg-Prkdc^{scid}Il2rg^{tm1Wjl}Rhbdf^{P159L}/SzJ mouse, wherein the NOD.Cg-Prkdc^{scid}Il2rg^{tm1Wjl}Rhbdf^{P159L}/SzJ mouse is characterized by hairless phenotype and increased growth of a xenogeneic tumor compared to a mouse of the same genetic background which expresses wild-type iRhom2 protein; providing a xenogeneic tumor cell obtained from a tumor of a human subject; administering the xenogeneic tumor cell obtained from a tumor of a human subject to the NOD.Cg-Prkdc^{scid}Il2rg^{tm1Wjl}Rhbdf^{P159L}/SzJ mouse, producing a NOD.Cg-Prkdc^{scid}Il2rg^{tm1Wjl}Rhbdf^{P159L}/SzJ mouse including a xenogeneic tumor cell obtained from a tumor of a human subject; administering a test substance to the NOD.Cg-Prkdc^{scid}Il2rg^{tm1Wjl}Rhbdf^{P159L}/SzJ mouse including a xenogeneic tumor cell obtained from a tumor of a human subject; assaying a response of a xenogeneic tumor cell obtained from a tumor of a human subject to the test substance following administration of the test substance to the NOD.Cg-Prkdc^{scid}Il2rg^{tm1Wjl}Rhbdf^{P159L}/SzJ mouse including a xenogeneic tumor cell obtained from a tumor of a human subject; and comparing the response to a standard to determine the effect of the test substance on a xenogeneic tumor cell obtained from a tumor of a human subject, wherein an inhibitory effect of the test substance on a xenogeneic tumor cell obtained from a tumor of a human subject identifies the test substance as an anti-tumor composition.

### BRIEF DESCRIPTION OF THE DRAWINGS

FIG. 1 shows a schematic diagram of mouse iRhom2 (SEQ ID NO:1), showing the N-terminal region (amino acids 1-373) and C-terminal region (amino acids 374-827) and indicating the relative location of amino acid 159;

FIG. 2A is a graphic output of a DNA sequencing instrument showing results of DNA sequencing of a region of the mouse wild-type (WT) Rhbdf2 gene encoding iRhom2 at amino acid 159 (top, arrow) compared with results of DNA sequencing of a region of the mouse p.P159L mutant Rhbdf2 gene encoding p.P159L mutant iRhom2 at amino acid 159 (bottom, arrow);

FIG. 2B is graphic output of a DNA sequencing instrument showing results of DNA sequencing of a region of the mouse p.I156T mutant Rhbdf2 gene encoding p.I156T mutant iRhom2 at amino acid 156 codon mutated from ATT (wild-type) to ACT (shaded C);

FIG. 2C is graphic output of a DNA sequencing instrument showing results of DNA sequencing of a region of the mouse p.D158N mutant Rhbdf2 gene encoding p.D158N mutant iRhom2 at amino acid 158 codon mutated from GAT (wild-type) to AAT (arrow);

FIG. 3 is an image of a 6-week-old mouse carrying the Rhbdf2 p.P159L mutation (NSG-Bald) and characterized by a hairless phenotype (left) and a normal white haired littermate control mouse (right) carrying a wildtype Rhbdf2 allele;

FIG. 4 is a graph showing increased xenogeneic tumor growth in an NSG mouse carrying the Rhbdf2 p.P159L mutation (NSG-Bald) and expressing iRhom2 with the p.P159L mutation in the N-terminal region;

FIG. 5A is a set of images showing the results of immunostaining of MDA-MB 231 human breast cancer cell-derived tumors for vascular marker CD31 in an NSG mouse;

FIG. 5B is a set of images showing the results of immunostaining of MDA-MB 231 human breast cancer cell-derived tumors for vascular marker CD31 in a mouse expressing iRhom2 with a mutation in the N-terminal region;

FIG. 6 is a comparison of mouse iRhom1 and iRhom2 amino acid sequences

FIG. 7 is an image showing that mice heterozygous for Rhbdf1 gene deletion (Rhbdf1^{+/-}) mice are normal in size (right) and mice homozygous for Rhbdf1 gene deletion (Rhbdf1^{-/-}) are smaller in size (left);

FIG. 8 is a graph showing that mice heterozygous for Rhbdf1 gene deletion (Rhbdf1^{+/-}) mice display normal percent survival (top line) while mice homozygous for Rhbdf1 gene deletion (Rhbdf1^{-/-}) die by 3-4 weeks of age (bottom line);

FIG. 9 is a set of three images of hematoxylin and eosin stained sections of hearts isolated from Rhbdf1^{-/-} homozygous mice with severe cardiac fibrosis (marked with “o”) which leads to death at around 3-4 weeks of age, scale bar 1 mm; and

FIG. 10 is an image of a hematoxylin and eosin stained section of a heart isolated from a Rhbdf1^{-/+} heterozygous mouse which shows no cardiac fibrosis, scale bar 1 mm.

### DETAILED DESCRIPTION OF THE INVENTION

Rhomboid proteins exist in almost all species. Rhomboid proteases are intramembrane serine proteases responsible for cleavage events important for cellular regulation. The active site of these intramembrane proteases is buried in the lipid bilayer of cell membranes, and they cleave other transmembrane proteins within their transmembrane domains.

Inactive rhomboids (iRhoms) are highly conserved intramembrane proteins that were previously thought to be proteolytically inactive. Wild-type iRhoms are characterized by a cytosolic N-terminal domain, a conserved cysteine-rich inactive rhomboid homology domain (IRHD) and a dormant proteolytic site lacking an active-site serine residue within the peptidase domain.

iRhoms participate in a diverse range of functions in a variety of species, including regulation of epidermal growth factor receptor (EGFR) signaling in Drosophila melanogaster, survival of human squamous epithelial cancer cells, misfolded protein clearance from endoplasmic reticulum membranes in mammalian cell lines, induction of migration in primary mouse keratinocytes, secretion of soluble TNFα in mice, and regulation of substrate selectivity of stimulated ADAM17-mediated metalloprotease shedding in mouse embryonic fibroblasts. EGF-like ligands may act as substrates for iRhom family members.

Additional aspects of iRhom function are continuing to be elucidated. iRhom2 knockout mice were found to be “viable and fertile, show no obvious defects, have a normal life-span, and exhibit a normal immune cell distribution” as described in McIlwain et al., Science, 2012, 335(6065):229-232.

Surprisingly, according to aspects of the present invention described herein it is found that one or more mouse iRhom2 mutations selected from p.I156T, p.D158N and p.P159L result in a hairless phenotype and an increase in growth of xenogeneic tumors in NSG mice. NSG mice are useful for study of xenotransplants of various types but they are characterized by extremely slow xenogeneic tumor growth and imaging xenogeneic tumors can be difficult due to hair on skin covering a xenogeneic tumor, such that these phenotypes provide significant advantages that contribute to development of anti-tumor drugs and treatments in humans as well as other mammals.

Accordingly, genetically a modified immunodeficient mouse is provided by the present invention wherein the genome of the immunodeficient mouse includes a mutated Rhbdf2 gene such that the genetically modified immunodeficient mouse express a mutant iRhom2 protein, wherein the mutant iRhom2 protein differs from wild-type iRhom2 protein due to one or more mouse iRhom2 mutations selected from p.I156T, p.D158N and p.P159L, and wherein the genetically modified immunodeficient mouse is characterized by a hairless phenotype and increased growth of xenogeneic tumor cells.

Genetically modified immunodeficient mice, methods and compositions of the present invention have various utilities such as, but not limited to, models of xenogeneic tumor cell growth, in vivo methods of assay of response of engrafted xenogeneic tumors to drugs and/or therapeutic treatments, testing of agents affecting the innate immune system and testing agents that affect signaling and activity relating to iRhom2.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer (Eds) 2002, Manipulating the Mouse Embryo: A Laboratory Manual, 3^{rd} edition, Cold Spring Harbor Laboratory Press, ISBN-10: 0879695919; and K. Turksen (Ed.), Embryonic stem cells: methods and protocols in Methods Mol Biol. 2002; 185, Humana Press; Current Protocols in Stem Cell Biology, ISBN: 9780470151808.

The singular terms “a,” “an,” and “the” are not intended to be limiting and include plural referents unless explicitly stated otherwise or the context clearly indicates otherwise.

The term “nucleic acid” as used herein refers to RNA or DNA molecules having more than one nucleotide in any form including single-stranded, double-stranded, oligonucleotide or polynucleotide. The term “nucleotide sequence” is used to refer to the ordering of nucleotides in an oligonucleotide or polynucleotide in a single-stranded form of nucleic acid.

The terms “duplex” and “double-stranded” are used to refer to nucleic acids characterized by binding interaction of complementary nucleotide sequences. A duplex includes a “sense” strand and an “antisense” strand. Such duplexes include RNA/RNA, DNA/DNA or RNA/DNA types of duplexes.

The term “oligonucleotide” is used herein to describe a nucleotide sequence having from 2-1000 linked nucleotides, while the term “polynucleotide” is used to describe a nucleotide sequence having more than 1000 nucleotides.

The term “nucleotide” is used herein as a noun to refer to individual nucleotides or varieties of nucleotides as opposed to a nucleotide sequence.

The term “genetically engineered mouse” as used herein refers to a mouse that contains one or more DNA modifications introduced into the individual mouse genome or mouse strain genome by means of molecular biology techniques, i.e., recombinant DNA technology. The term “genetically engineered mouse” encompasses offspring of a mouse that contains one or more DNA modifications introduced into the individual mouse genome wherein those offspring also contain the one or more DNA modifications.

The term “wild-type” refers to an unmutated mouse, protein or nucleic acid.

The mouse wild-type Rhbdf2 gene encodes an inactive rhomboid protease iRhom2, containing a cytosolic N-terminal region (amino acids 1-347), a conserved homology domain and a dormant peptidase domain.

Wild-type mouse iRhom2 protein has the amino acid sequence shown herein as SEQ ID NO: 1, encoded by wild-type Rhbdf2 gene sequence SEQ ID NO:2.

Wild-type mouse iRhom1 protein has the amino acid sequence shown herein as SEQ ID NO:3.

The terms “expression,” “expressing,” “expresses” and grammatical equivalents refer to transcription of a gene to produce a corresponding mRNA and/or translation of the mRNA to produce the corresponding protein. The terms “express,” “expression,” “expressing” and “expresses” with reference to the mutated Rhbdf2 gene refer to transcription of the mutated Rhbdf2 gene to produce a corresponding mRNA and/or translation of the mRNA to produce a corresponding mutant iRhom2 protein.

In particular aspects of the present invention, a mutation is introduced into the Rhbdf2 gene of an immunodeficient mouse, generating a genetically engineered immunodeficient mouse characterized by expression of a mutant iRhom2 protein wherein the mutant iRhom2 protein differs from wild-type iRhom2 protein due to one or more mouse iRhom2 mutations selected from p.I156T, p.D158N and p.P159L, and further characterized by a hairless phenotype and increased xenogeneic tumor growth compared to immunodeficient mice of the of the same genetic background which express wild-type iRhom2 protein.

The term “hairless phenotype” as used herein refers to a genetically engineered immunodeficient mouse expressing an iRhom2 protein with one or more mutations in the N-terminal region, wherein the genetically engineered immunodeficient mouse has 95% or less hair compared to a mouse of the same genetic background which expresses wild-type iRhom2. According to particular aspects, a genetically engineered NSG mouse expressing an iRhom2 protein with one or more mouse iRhom2 mutations selected from p.I156T, p.D158N and p.P159L, has 95% or less hair compared to a mouse of the same genetic background which expresses wild-type iRhom2.

The term “increased xenogeneic tumor growth” as used herein refers to a characteristic of a genetically engineered immunodeficient mouse expressing an iRhom2 protein with one or more mutations in the N-terminal region, wherein a xenogeneic tumor in the genetically engineered immunodeficient mouse increases in volume more quickly compared to a xenogeneic tumor in a mouse of the same genetic background which expresses the corresponding wild-type iRhom2. According to particular aspects, a genetically engineered NSG mouse expressing an iRhom2 protein with one or more mouse iRhom2 mutations selected from p.I156T, p.D158N and p.P159L, is characterized by increased xenogeneic tumor growth compared to a mouse of the same genetic background which expresses wild-type iRhom2.

The term “immunodeficient mouse” refers to a mouse characterized by one or more of: a lack of functional immune cells, such as T cells and B cells; a DNA repair defect; a defect in the rearrangement of genes encoding antigen-specific receptors on lymphocytes; and a lack of immune functional molecules such as IgM, IgG1, IgG2a, IgG2b, IgG3 and IgA. Immunodeficient mice can be characterized by one or more deficiencies in a gene involved in immune function, such as Rag1 and Rag2 (Oettinger, M. A et al., Science, 248:1517-1523, 1990; and Schatz, D. G. et al., Cell, 59:1035-1048, 1989) Immunodeficient mice may have any of these or other defects which result in abnormal immune function in the mice.

Particularly useful immunodeficient mouse strains are NOD.Cg-Prkdc^{scid} Il2rg^{tm1Wjl}Rhbdf^{P159L}/SzJ, commonly referred to as NOD scid gamma (NSG) mice, described in detail in Shultz L D et al, 2005, J. Immunol, 174:6477-89, NOD.Cg-Rag1^{tm1Mom} Il2rg^{tm1Wjl}/SzJ, Shultz L D et al, 2008 Clin Exp Immunol 154(2):270-84 commonly referred to as NRG mice, and NOD.Cg-Prkdc^{scid} Il2rg^{tm1Sug}/JicTac, commonly referred to as NOG mice, described in detail in Ito, M. et al., Blood 100, 3175-3182 (2002).

The terms “NOD scid gamma” and “NSG” are used interchangeably herein to refer to a well-known immunodeficient mouse strain NOD.Cg-Prkdc^{scid} Il2rg^{tm1Wjl}/SzJ. NSG mice combine multiple immune deficits from the NOD/ShiLtJ background, the severe combined immune deficiency (scid) mutation, and a complete knockout of the interleukin-2 receptor gamma chain. As a result, NSG mice lack mature T, B and NK cells, and are deficient in cytokine signaling. NSG mice are characterized by lack of IL2R-γ (gamma c) expression, no detectable serum immunoglobulin, no hemolytic complement, no mature T lymphocytes, and no mature natural killer cells.

The term “severe combined immune deficiency (SCID)” refers to a condition characterized by absence of T cells and lack of B cell function.

Common forms of SCID include: X-linked SCID which is characterized by gamma chain gene mutations in the IL2RG gene and the lymphocyte phenotype T(−) B(+) NK(−); and autosomal recessive SCID characterized by Jak3 gene mutations and the lymphocyte phenotype T(−) B(+) NK(−), ADA gene mutations and the lymphocyte phenotype T(−) B(−) NK(−), IL-7R alpha-chain mutations and the lymphocyte phenotype T(−) B(+) NK(+), CD3 delta or epsilon mutations and the lymphocyte phenotype T(−) B(+) NK(+), RAG1/RAG2 mutations and the lymphocyte phenotype T(−) B(−) NK(+), Artemis gene mutations and the lymphocyte phenotype T(−) B(−) NK(+), CD45 gene mutations and the lymphocyte phenotype T(−) B(+) NK(+).

A genetically modified immunodeficient mouse according to aspects of the present invention has the severe combined immunodeficiency mutation (Prkdc^{scid}), commonly referred to as the scid mutation. The scid mutation is well-known and located on mouse chromosome 16 as described in Bosma, et al., Immunogenetics 29:54-56, 1989. Mice homozygous for the scid mutation are characterized by an absence of functional T cells and B cells, lymphopenia, hypoglobulinemia and a normal hematopoetic microenvironment. The scid mutation can be detected, for example, by detection of markers for the scid mutation using well-known methods, such as PCR or flow cytometry.

A genetically modified immunodeficient mouse according to aspects of the present invention has an IL2 receptor gamma chain deficiency. The term “IL2 receptor gamma chain deficiency” refers to decreased IL2 receptor gamma chain. Decreased IL2 receptor gamma chain can be due to gene deletion or mutation. Decreased IL2 receptor gamma chain can be detected, for example, by detection of IL2 receptor gamma chain gene deletion or mutation and/or detection of decreased IL2 receptor gamma chain expression using well-known methods.

Genetically modified immunodeficient mice having severe combined immunodeficiency or an IL2 receptor gamma chain deficiency in combination with severe combined immunodeficiency are provided according to aspects of the present invention whose genome includes a mutated Rhbdf2 gene.

Genetically modified immunodeficient mice having the scid mutation or an IL2 receptor gamma chain deficiency in combination with the scid mutation are provided according to aspects of the present invention whose genome includes a mutated Rhbdf2 gene such that the mice express mutant iRhom2 protein which differs from wild-type iRhom2 protein due to one or more mouse iRhom2 mutations selected from p.I156T, p.D158N and p.P159L, and wherein the mice are characterized by a hairless phenotype and increased growth of xenogeneic tumor cells.

Genetically modified immunodeficient mice having the scid mutation or an IL2 receptor gamma chain deficiency in combination with the scid mutation are provided according to aspects of the present invention whose genome includes a mutated Rhbdf2 gene such that the mice express mutant iRhom2 protein which differs from wild-type iRhom2 protein due to one or more mouse iRhom2 mutations selected from p.I156T, p.D158N and p.P159L, and wherein the mice are characterized by a hairless phenotype and increased growth of xenogeneic tumor cells.

Genetically modified NSG mice are provided according to aspects of the present invention whose genome includes a mutated Rhbdf2 gene such that the mice express mutant iRhom2 protein which differs from wild-type iRhom2 protein due to one or more mouse iRhom2 mutations selected from p.I156T, p.D158N and p.P159L, and wherein the mice are characterized by a hairless phenotype and increased growth of xenogeneic tumor cells.

“Mutation” of the Rhbdf2 gene refers to genetic modification of the gene such that a mouse having the mutation of the Rhbdf2 gene expresses a mutant iRhom2 protein which differs from wild-type iRhom2 protein due to one or more mutations in the N-terminal region, and wherein the mouse is characterized by a hairless phenotype and increased growth of xenogeneic tumor cells.

According to aspects of the present invention, a genetically modified immunodeficient mouse includes a mutated Rhbdf2 gene such that the genetically modified immunodeficient mouse expresses mutant iRhom2 protein which differs from wild-type iRhom2 protein due to in frame deletion of at least a portion of the N-terminal region extending from amino acid 1-373 of SEQ ID NO:1, and wherein the genetically modified immunodeficient mouse is characterized by a hairless phenotype and increased growth of xenogeneic tumor cells. Amino acids 374-827 of the mutant iRhom2 are identical to or substantially similar to the wild-type iRhom2 protein of SEQ ID NO: 1.

According to aspects of the present invention, a genetically modified NSG mouse includes a mutated Rhbdf2 gene such that the genetically modified NSG mouse expresses mutant iRhom2 protein which differs from wild-type iRhom2 protein due to in frame deletion of at least a portion of the N-terminal region extending from amino acid 1-373 of SEQ ID NO:1, and wherein the genetically modified NSG mouse is characterized by a hairless phenotype and increased growth of xenogeneic tumor cells. Amino acids 374-827 of the mutant iRhom2 are identical to or substantially similar to the wild-type iRhom2 protein of SEQ ID NO: 1.

The term “substantially similar” with reference to amino acids 374-827 the wild-type iRhom2 protein of SEQ ID NO:1 refers to an amino acid sequence having identity of 70%, 75%, 80%, 81%, 82%, 83%, 84%, 85%, 86%, 87%, 88%, 89%, 90%, 91%, 92%, 93%, 94%, 95%, 96%, 97%, 98% or 99% or greater while retaining the function of amino acids 374-827 of the wild-type iRhom2.

According to aspects of the present invention, genetically modified immunodeficient mouse having includes a mutated Rhbdf2 gene such that the mouse expresses mutant iRhom2 protein which differs from wild-type iRhom2 protein of SEQ ID NO:1 due to one, two or three mutations in the N-terminal region at amino acid isoleucine 156, aspartic acid 158 and proline 159, wherein amino acids 374-827 of the mutant iRhom2 are identical to or substantially similar to the wild-type iRhom2 protein of SEQ ID NO:1 and wherein the mouse is characterized by a hairless phenotype and increased growth of xenogeneic tumor cells.

One, two or three amino acids selected from: isoleucine 156, aspartic acid 158 and proline 159 of iRhom2 can be mutated to any other amino acid, or deleted, by genetic modification of the mouse Rhbdf2 gene to produce a genetically modified immunodeficient mouse having a mutated Rhbdf2 gene, wherein the mouse expresses a mutant iRhom2 protein having one or more mouse iRhom2 mutations selected from p.I156T, p.D158N and p.P159L, wherein amino acids 374-827 of the mutant iRhom2 are identical to or substantially similar to the wild-type iRhom2 protein of SEQ ID NO:1 and wherein the mouse is characterized by a hairless phenotype and increased growth of xenogeneic tumor cells.

According to aspects of the present invention, codon 156 of the Rhbdf2 gene sequence encoding iRhom2 is mutated from ATT (wild-type) to ACT or other substitutions or deletion of the codon such that the open reading frame is intact.

According to aspects of the present invention, codon 158 of the Rhbdf2 gene sequence encoding iRhom2 is mutated from GAT to AAT or AAC or other substitutions or deletion of the codon such that the open reading frame is intact.

According to aspects of the present invention, codon 159 of the Rhbdf2 gene sequence encoding iRhom2 is mutated from CCA to CTA or other substitutions or deletion of the codon such that the open reading frame is intact.

It is appreciated that due to the degenerate nature of the genetic code, more than one nucleic acid sequence encodes a particular iRhom2 polypeptide, and that such nucleic acid sequences produce the desired iRhom2.

A genetically modified NSG mouse is provided according to aspects of the present invention whose genome includes a mutated Rhbdf2 gene is a NOD.Cg-Prkdc^{scid}Il2rg^{tm1Wjl}Rhbdf^{P159L}/SzJ (NSG-BALDP159L) mouse with a mutated Rhbdf2 gene at the proline 159, wherein the mouse is characterized by a hairless phenotype and increased growth of xenogeneic tumor cells.

A genetically modified NSG mouse is provided according to aspects of the present invention whose genome includes a mutated Rhbdf2 gene is a NOD.Cg-Prkdc^{scid}Il2rg^{tm1Wjl}Rhbdf^{P159L}/SzJ (NSG-BALDP159X) mouse with a mutated Rhbdf2 gene at the proline 159, where X is any amino acid, wherein the mouse is characterized by a hairless phenotype and increased growth of xenogeneic tumor cells.

A genetically modified NSG mouse is provided according to aspects of the present invention whose genome includes a mutated Rhbdf2 gene is a NOD.Cg-Prkdc^{scid}Il2rg^{tm1Wjl}Rhbdf^{P159L}/SzJ (NSG-BALDI156T) mouse with a mutated Rhbdf2 gene at the isoleucine 156, wherein the mouse is characterized by a hairless phenotype and increased growth of xenogeneic tumor cells.

A genetically modified NSG mouse is provided according to aspects of the present invention whose genome includes a mutated Rhbdf2 gene is a NOD.Cg-Prkdc^{scid}Il2rg^{tm1Wjl}Rhbdf^{P159L}/SzJ (NSG-BALDP156X) mouse with a mutated Rhbdf2 gene at the isoleucine 156, where X is any amino acid, wherein the mouse is characterized by a hairless phenotype and increased growth of xenogeneic tumor cells.

A genetically modified NSG mouse is provided according to aspects of the present invention whose genome includes a mutated Rhbdf2 gene is a NOD.Cg-Prkdc^{scid}Il2rg^{tm1Wjl}Rhbdf^{P159L}/SzJ (NSG-BALDD158N) mouse with a mutated Rhbdf2 gene at the aspartic acid 158, wherein the mouse is characterized by a hairless phenotype and increased growth of xenogeneic tumor cells.

A genetically modified NSG mouse is provided according to aspects of the present invention whose genome includes a mutated Rhbdf2 gene is a NOD.Cg-Prkdc^{scid}Il2rg^{tm1Wjl}Rhbdf^{P159L}/SzJ (NSG-BALDP158X) mouse with a mutated Rhbdf2 gene at the aspartic acid 158, where X is any amino acid, wherein the mouse is characterized by a hairless phenotype and increased growth of xenogeneic tumor cells.

While aspects of inventive genetically modified mice and their uses are described with particular reference to one or more mouse iRhom2 mutations selected from p.I156T, p.D158N and p.P159L, one or more different or additional mutations in the N-terminal region of iRhom2 are contemplated and it is intended to embrace all such alternatives, modifications and variations that fall within the spirit and broad scope of the present disclosure and claims.

Any of various methods can be used to mutate the Rhbdf2 gene to produce a genetically modified immunodeficient mouse whose genome includes a mutation of the Rhbdf2 gene. The Rhbdf2 gene is mutated in the genome of genetically modified immunodeficient mice according to standard methods of genetic engineering such as, but not limited to, genome editing, chemical mutagenesis, irradiation, homologous recombination and transgenic expression of antisense RNA.

Methods for generating genetically modified animals whose genome includes a mutated gene include, but are not limited to, those described in J. P. Sundberg and T. Ichiki, Eds., Genetically Engineered Mice Handbook, CRC Press; 2006; M. H. Hofker and J. van Deursen, Eds., Transgenic Mouse Methods and Protocols, Humana Press, 2002; A. L. Joyner, Gene Targeting: A Practical Approach, Oxford University Press, 2000; Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 978047015180; Meyer et al. PNAS USA, vol. 107 (34), 15022-15026.

Genetic Modification Methods

Homology-based recombination gene modification strategies can be used, such as homing endonucleases, integrases, meganucleases, transposons, nuclease-mediated processes using a zinc finger nuclease (ZFN), a Transcription Activator-Like (TAL), a Clustered Regularly Interspaced Short Palindromic Repeats (CRISPR)-Cas, or a Drosophila Recombination-Associated Protein (DRAP) approach. Briefly, the process includes introducing into ES or iPS cells RNA molecules encoding a targeted TALEN or ZFN or CRISPR or DRAP and at least one oligonucleotide, then selecting for an ES or iPS cell with the corrected gene.

Mutation of the gene may be accomplished directly in the fertilized oocyte (zygotes) or embryo. For this, homing endonucleases, integrases, meganucleases, transposons, nuclease-mediated processes, such as zinc finger nuclease, TALEN, CRISPR-Cas or DRAP can be applied. Preferred approaches are TALEN, ZFN, CRISPR-Cas or DRAP. Briefly, the method includes introducing into a fertilized oocyte or an embryo or a cell at least one nucleic acid molecule encoding a targeted TALEN, ZFN, CRISPR-Cas or DRAP and, at least one oligonucleotide. The method further includes incubating the fertilized oocyte, embryo or cell to allow expression of the TALEN, ZFN, CRISPR-Cas or DRAP, wherein a double-stranded break introduced into the targeted chromosomal sequence by the TALEN, ZFN, CRISPR-Cas or DRAP is repaired by a homology-directed DNA repair process. The nucleic acid encoding TALEN, ZFN, CRISPR-Cas or DRAP can be DNA, as an expression vector, or RNA. Instead of nucleic acid encoding TALEN, ZFN, CRISPR-Cas or DRAP, a TALEN, ZFN, CRISPR-Cas or DRAP protein may be delivered to the fertilized oocyte or an embryo or a cell. The DRAP technology has been described in U.S. Pat. No. 6,534,643, U.S. Pat. No. 6,858,716 and U.S. Pat. No. 6,830,910 and Watt et al, 2006.

As used herein, the terms “target site” and “target sequence” refer to a nucleic acid sequence that defines a portion of a chromosomal sequence to be edited and to which a nuclease is engineered to recognize and bind, provided sufficient conditions for binding exist.

Nucleases

Nucleases, including TALEN, ZFN, and homing endonucleases such as I-SceI, engineered to specifically bind to target sites have been successfully used for genome modification in a variety of different species.

TAL (Transcription Activator-Like) Effectors

The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like (TAL) effectors or TALE (transcription activator-like effector) which mimic plant transcriptional activators and manipulate the plant transcript, see Kay et al 2007, Science, 318:648-651. These proteins contain a DNA binding domain and a transcriptional activation domain. One of the most well characterized TAL-effectors is AvrBs3 from Xanthomonas campestris pv. vesicatoria, (see Bonas et al 1989, Mol Gen Genet 218: 127-136 and WO2010079430). TAL effectors contain a centralized domain of tandem repeats, each repeat containing approximately 34 amino acids, which are key to the DNA binding specificity of these proteins. In addition, they contain a nuclear localization sequence and an acidic transcriptional activation domain, for a review see Schornack et al 2006, J Plant PhysioI163(3): 256-272; Scholze and Boch 2011, Curr Opin Microbiol, 14:47-53. In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI 1000 and in the biovar 4 strain RS1000, see Heuer et al. 2007, Appl and Envir Micro 73(13): 4379-4384. These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TAL effectors depends on the sequences found in the tandem repeats. The repeated sequence includes approximately 102 bp and the repeats are typically 91-100% homologous with each other (Bonas et al, 1989, Mol Gen Genet 218: 127-136). Polymorphism of the repeats is usually located at positions 12 and 13 and there appears to be a one-to-one correspondence between the identity of the hypervariable diresidues at positions 12 and 13 with the identity of the contiguous nucleotides in the TAL-effector's target sequence, see Moscou and Bogdanove 2009, Science 326: 1501; and Boch et al 2009, Science 326:1509-1512. The two hypervariable residues are known as repeat variable diresidues (RVDs), whereby one RVD recognizes one nucleotide of DNA sequence and ensures that the DNA binding domain of each TAL-effector can target large recognition sites with high precision (15-30 nt). Experimentally, the code for DNA recognition of these TAL-effectors has been determined such that an HD sequence at positions 12 and 13 leads to a binding to cytosine (C), NG binds to T, NI to A, C, G or T, NN binds to A or G, and IG binds to T. These DNA binding repeats have been assembled into proteins with new combinations and numbers of repeats, to make artificial transcription factors that are able to interact with new sequences and activate the expression of a reporter gene in plant cells (Boch et al 2009, Science 326:1509-1512). These DNA binding domains have now been shown to have general applicability in the field of targeted genomic editing or targeted gene regulation in all cell types (Gaj et al, 2013). Moreover, engineered TAL effectors have been shown to function in association with exogenous functional protein effector domains such as a nuclease, not naturally found in natural Xanthomonas TAL-effect or proteins in mammalian cells. TAL nucleases (TALNs or TALENs) can be constructed by combining TALs with a nuclease, e.g. FokI nuclease domain at the N-terminus or C-terminus, Kim et al. 1996, PNAS 93:1156-1160; Christian et al 2010, Genetics 186:757-761; Li et al, 2011; and Miller et al, 2011. The functionality of TALENs to cause deletions by NHEJ has been shown in rat, mouse, zebrafish, Xenopus, medaka, rat and human cells, Ansai et al, 2013; Carlson et al, 2012; Hockemeyer et al, 2011; Lei et al, 2012; Moore et al, 2012; Stroud et al, 2013; Sung et al, 2013; Wefers et al, 2013.

For TALEN, methods of making such are further described in the U.S. Pat. No. 8,420,782, U.S. Pat. No. 8,450,471, U.S. Pat. No. 8,450,107, U.S. Pat. No. 8,440,432, U.S. Pat. No. 8,440,431 and US patent applications US20130137161, US20130137174.

Other useful endonucleases may include, for example, HhaI, HindIII, NotI, BbvCI, EcoRI, Bg/I, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

In some embodiments, the TALEN may further include a nuclear localization signal or sequence (NLS). A NLS is an amino acid sequence that facilitates targeting the TALEN nuclease protein into the nucleus to introduce a double stranded break at the target sequence in the chromosome.

Nuclear localization signals are known in the art, see, for example, Makkerh et al. 1996, Curr Biol. 6:1025-1027. NLS include the sequence PKKKRKV (SEQ ID NO: 5) from SV40 Large T-antigen, Kalderon 1984, Cell 39: 499-509; RPAATKKAGQAKKK (SEQ ID NO: 6) from nucleoplasmin, Dingwallet al., 1988, J Cell Biol. 107, 841-9. Further examples are described in McLane and Corbett 2009, IUBMB Life 61, 697-70; Dopie et al. 2012, PNAS 109, E544-E552.

The cleavage domain may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388. Additional enzymes that cleave DNA are known, e.g., SI Nuclease; mung bean nuclease; pancreatic DNase I; micrococcal nuclease; yeast HO endonuclease. See also Linn et al. (eds.) Nucleases, Cold Spring Harbor Laboratory Press, 1993. One or more of these enzymes, or functional fragments thereof, may be used as a source of cleavage domains.

Zinc Finger-Mediated Genome Editing

The use of zinc finger nucleases (ZFN) for gene editing, especially for creating deletions, has been well established. For example see Carbery et al, 2010; Cui et al, 2011; Hauschild et al, 2011; Orlando et al, 2010; and Porteus & Carroll, 2005. ZFNs can be used to generate knockouts by introducing non-homologous end joining (NHEJ)-mediated deletions or for targeted insertion via a homology-directed repair process.

Components of the zinc finger nuclease-mediated process include a zinc finger nuclease with a DNA binding domain and a cleavage domain. Such are described for example in Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr Opin. Biotechnol. 12:632-637; and Choo et al. (2000) Curr Opin. Struct. Biol. 10:411-416; and U.S. Pat. Nos. 6,453,242 and 6,534,261. Methods to design and select a zinc finger binding domain to a target sequence are known in the art, see for example Biochemistry 2002, 41, 7074-7081; U.S. Pat. Nos. 6,607,882; 6,534,261 and 6,453,242. In some embodiments, the zinc finger nuclease may further include a nuclear localization signal or sequence (NLS). A NLS is an amino acid sequence that facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027. The cleavage domain may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388. Additional enzymes that cleave DNA are known (e.g., SI Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains. A cleavage domain also may be derived from an enzyme or portion thereof, as described above, that requires dimerization for cleavage activity. Two zinc finger nucleases may be required for cleavage, as each nuclease includes a monomer of the active enzyme dimer. Alternatively, a single zinc finger nuclease may include both monomers to create an active enzyme dimer. Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) PNAS 89:4275-4279; Li et al. (1993) PNAS 90:2764-2768; Kim et al. (1994) PNAS 91:883-887; Kim et al. (1994) J. Biol. Chem. 269:31, 978-31, 982. Thus, a zinc finger nuclease may include the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31: 418-420. An exemplary Type IIS restriction enzyme, whose cleavage domain is separable from the binding domain, is FokI. This particular enzyme is active as a dimer (Bitinaite et al. 1998, PNAS 95: 10,570-10,575). Accordingly, for the purposes of the present disclosure, the portion of the Fold enzyme used in a zinc finger nuclease is considered a cleavage monomer. Thus, for targeted double stranded cleavage using a FokI cleavage domain, two zinc finger nucleases, each including a FokI cleavage monomer, may be used to reconstitute an active enzyme dimer. Alternatively, a single polypeptide molecule containing a zinc finger binding domain and two FokI cleavage monomers may also be used. In certain embodiments, the cleavage domain may include one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537 and 538 of FokI are all targets for influencing dimerization of the Fold cleavage half-domains. Exemplary engineered cleavage monomers of FokI that form obligate heterodimers include a pair in which a first cleavage monomer includes mutations at amino acid residue positions 490 and 538 of Fold and a second cleavage monomer that includes mutations at amino-acid residue positions 486 and 499. Thus, in one embodiment, a FokI mutation at amino acid position 490 replaces Glu (E) with Lys (K); a mutation at amino acid residue 538 replaces Ile (I) with Lys (K); a mutation at amino acid residue 486 replaces Gln (Q) with Glu (E); and a mutation at position 499 replaces Ile (I) with Lys (K). Specifically, the engineered cleavage monomers of FokI may be prepared by mutating positions 490 from E to K and 538 from I to K in one cleavage monomer to produce an engineered cleavage monomer designated “E490K:I538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (FokI) as described in U.S. Patent Publication No. 20050064474.

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100 or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45 or 50 nucleotides away from the site of integration. In yet other embodiments, the double stranded break may be up to 50, 100 or 1000 nucleotides away from the site of integration.

CRISPR-Cas System

CRISPRs (Clustered Regularly Interspaced Short Palindromic Repeats) are loci containing multiple short direct repeats that are found in the genomes of approximately 40% of sequenced bacteria and 90% of sequenced archaea and confer resistance to foreign DNA elements, see Horvath, 2010, Science 327: 167-170; Barrangou et al, 2007; and Makarova et al, 2011. CRISPR repeats range in size from 24 to 48 base pairs. They usually show some dyad symmetry, implying the formation of a secondary structure such as a hairpin, but are not truly palindromic. CRISPR repeats are separated by spacers of similar length.

The CRISPR-associated (cas) genes are often associated with CRISPR repeat-spacer arrays. More than forty different Cas protein families have been described (Haft et al. 2005, PLoS Comput Biol. 1 (6): e60). Particular combinations of cas genes and repeat structures have been used to define 8 CRISPR subtypes, some of which are associated with an additional gene module encoding repeat-associated mysterious proteins (RAMPs).

There are diverse CRISPR systems in different organisms, and one of the simplest is the type II CRISPR system from Streptococcus pyogenes: only a single gene encoding the Cas9 protein and two RNAs, a mature CRISPR RNA (crRNA) and a partially complementary trans-acting RNA (tracrRNA), are necessary and sufficient for RNA-guided silencing of foreign DNAs (Gasiunas et al, 2012; Jinek et al, 2012). Maturation of crRNA requires tracrRNA and RNase III (Deltcheva et al, 2011). However, this requirement can be bypassed by using an engineered small guide RNA (sgRNA) containing a designed hairpin that mimics the tracrRNA-crRNA complex (Jinek et al., 2012). Base pairing between the sgRNA and target DNA causes double-strand breaks (DSBs) due to the endonuclease activity of Cas9. Binding specificity is determined by both sgRNA-DNA base pairing and a short DNA motif (protospacer adjacent motif [PAM] sequence: NGG) juxtaposed to the DNA complementary region (Marraffini & Sontheimer, 2010). For example, the CRISPR system requires a minimal set of two molecules, the Cas9 protein and the sgRNA, and therefore can be used as a host-independent gene-targeting platform. Recently, it has been demonstrated that the Cas9/CRISPR can be harnessed for site-selective RNA-guided genome editing (Carroll, 2012; Chang et al, 2013; Cho et al, 2013; Cong et al, 2013; Hwang et al, 2013; Jiang et al, 2013; Mali et al, 2013; Qi et al, 2013; Shen et al, 2013; Wang et al, 2013). Wang et al. 2013 have shown that a targeted insertion is possible with the CRISPR/Cas9system when combining it with oligonucleotides.

A nucleic acid encoding one or more nucleases and/or one or more other peptides or proteins, such as TALs, can be cloned into an expression vector for transformation into prokaryotic or eukaryotic cells and expression of the encoded peptides and/or protein(s). As used herein, “expression vectors” are defined as polynucleotides which, when introduced into an appropriate host cell, e.g. an expression system, can be transcribed and translated into a polypeptide(s). An in vivo “expression system” is a suitable host cell containing an expression vector that can function to yield a desired expression product. Expression vectors may also be used to produce the encoded proteins in vitro, such as in in vitro expression systems.

Expression vectors can be prokaryotic vectors, e.g., plasmids, or shuttle vectors, insect vectors, or eukaryotic vectors.

Nuclease expression constructs can be readily designed using methods known in the art. See, e.g., US Patent Publications 20030232410; 20050208489; 20050026157; 20050064474; 20060188987; 20060063231; 20080182332; 2009011188 and International Publication WO 07/014,275. Expression of the nuclease may be under the control of a constitutive promoter or an inducible promoter. Additional suitable bacterial and eukaryotic promoters are well known in the art and described, e.g., in Sambrook et al., Molecular Cloning, A Laboratory Manual (2nd ed. 1989; 3rd ed., 2001); Kriegler, Gene Transfer and Expression: A Laboratory Manual (1990); and Ausubel et al., Current Protocols in Molecular Biology. Bacterial expression systems for expressing the protein are available in, e.g., E. coli, Bacillus sp., and Salmonella, Palva et al. (1983) Gene 22:229-235.

In addition to the promoter, the expression vector typically contains a transcription unit or expression cassette that contains all the additional elements required for the expression of the nucleic acid in host cells, either prokaryotic or eukaryotic, for example signal sequences, enhancer elements, and transcription termination sequences. A typical expression cassette thus contains a promoter operably linked, e.g., to a nucleic acid sequence encoding the nuclease, and signals required, e.g., for efficient polyadenylation of the transcript, transcriptional termination, ribosome binding sites, or translation termination. Additional elements of the cassette may include, e.g., enhancers, heterologous splicing signals, and/or a nuclear localization signal (NLS).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, cytomegalovirus immediate early promoter; herpes simplex virus thymidine kinase promoter; early and late SV40 promoters; phosphoglycerate kinase (PGK) promoter; promoter present in long terminal repeats from a retrovirus; mouse metallothionein-I promoter; and various art-known tissue specific promoters.

In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL 10 promoter, an ADH2 promoter, a PH05 promoter, a CUP 1 promoter, a GAL7 promoter, a MET25 promoter, a MET3 promoter, a CYC1 promoter, a HIS 3 promoter, an ADH1 promoter, a PGK promoter, a GAPDH promoter, an ADC1 promoter, a TRP 1 promoter, a URA3 promoter, a LEU2 promoter, an ENO promoter, a TP1 promoter, and AOX1, e.g., for use in Pichia. Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter, see e.g., U.S. Patent Publication No. 20040131637; a pagC promoter, see Pulkkinen and Miller, J. Bacteriol, 1991: 173(1): 86-93; Alpuche-Aranda et al., PNAS, 1992; 89(21): 10079-83; a nirB promoter, see Harborne et al. (1992) Mol. Micro. 6:2805-2813; and the like, see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2; see, e.g., WO96/17951); an actA promoter, see, e.g., Shetron-Rama et al. (2002) Infect. Immun. 70: 1087-1096; an rps M promoter; see, e.g., Valdivia and Falkow (1996). Mol. Microbiol. 22:367); a Tet (Tetracycline) promoter, see, e.g., Hillen, W. and Wissmann, A. (1989) in Saenger, W. and Heinemann, U. (eds), Topics in Molecular and Structural Biology, Protein-Nucleic Acid Interaction. Macmillan, London, UK, Vol. 10, pp. 143-162; an SP6 promoter, see, e.g., Melton et al. (1984) Nucl. Acids Res. 12:7035; and the like. Suitable strong promoters for use in prokaryotes such as Escherichia coli include, but are not limited to Trc, Tac, T5, T7, and pLambda. Non-limiting examples of operators for use in bacterial host cells include a lactose promoter operator (Lac1 repressor protein changes conformation when contacted with lactose, thereby preventing the Lac1 repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator, see for example deBoer et al. (1983) PNAS 80:21-25.

Kits for such expression systems are commercially available. Eukaryotic expression systems for mammalian cells, are well known by those of skill in the art and are also commercially available.

Any of the well-known procedures for introducing foreign nucleotide sequences into host cells may be used. These include the use of calcium phosphate transfection, polybrene, protoplast fusion, electroporation, ultrasonic methods (e.g., sonoporation), liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

For example, Cas9 and dCas9 genes are cloned from the vectors pMJ806 and pMJ841 as described in Jinek et al., 2012. The genes are PCR amplified and inserted into a vector containing a Tc-inducible promoter PLtetO-1, (Lutz and Bujard, 1997, Nucleic Acids Res. 25:1203-10), a chloramphenicol-selectable marker, and a p15A replication origin. The sgRNA template is cloned into a vector containing a minimal synthetic promoter (J23119) with an annotated transcription start site, an ampicillin-selectable marker, and a ColE1 replication origin. Inverse PCR is used to generate sgRNA cassettes with new 20 bp complementary regions. Expression systems are described for example in Cong et al, 2013; and Jinek et al, 2012.

Donor oligonucleotides are used in combination with gene editing systems including TAL, ZFN, CRISPR, and DRAP according to aspects of the present invention.

The method for editing chromosomal sequences of the Rhbdf2 gene to produce a mutation includes introducing at least one donor oligonucleotide including a sequence to introduce a mutation of the Rhbdf2 gene into a fertilized oocyte, an embryo or cell. A donor oligonucleotide includes at least three components: the sequence coding the sequence of the Rhbdf2 mutation, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor oligonucleotide will be DNA. The donor oligonucleotide may be a DNA plasmid, a linear piece of DNA, a PCR fragment, a naked nucleic acid, a single strand nucleic acid, a synthetic oligonucleotide or a nucleic acid complexed with a delivery vehicle such as a liposome or poloxamer. In a preferred embodiment, the donor oligonucleotide is single stranded.

Particular oligonucleotides useful to mutate the mouse Rhbdf2 gene are provided by the present invention.

A donor oligonucleotide for introduction of point mutation p.P159L in wild-type Rhbdf2 gene sequence (SEQ ID NO:2) is:

(SEQ ID NO: 4)
CGTGCAAGATGCCCAAGGTGGGCCCCCTGGAGGTGATGGGCAGCAAGCGG
CTCTCCCAGGGTCTGGGCAACATTGTTCACCCACATCTCTTGCAGATTGT
GGATCTACTGGCTCGGGGTAGGGCCTTCCGCCATCCAGATGAGGTGGACC
GGCCTCACGCTGCCCACCCACCTCTGACTCCAGGGGTCCTGTCTCTCAC.

Variants of the donor oliognucleotide of SEQ ID NO:4 may be used to introduce the point mutation p.P159L in wild-type Rhbdf2 gene sequence. For example, donor oligonucleotide variants used to introduce point mutation p.P159L in wild-type Rhbdf2 gene sequence to produce a mutant iRhom2 with mutations in the N-terminus in addition to P159L.

According to aspects of the present invention a donor oligonucleotide has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity, or greater identity, to any of SEQ ID NO:4, wherein the variant encodes an amino acid sequence identical to the amino acid sequence encoded by SEQ ID NO:4.

The donor oligonucleotide for introduction of point mutation p.P159L (SEQ ID NO:4) was also found to be operative to introduce point mutations p.I156T and p.D158N. Further, the donor oligonucleotide of SEQ ID NO:4 was also found to be operative to introduce multiple point mutations into the genome of immunodeficient mice, particularly combinations of two or more of p.P159L, p.I156T and p.D158N; or all three of these mutations.

Donor nucleotide variants include those which are not identical to those disclosed herein but which, due to the degeneracy of the genetic code, encode the desired portion of wild-type or mutant iRhom2.

When comparing a reference protein to a putative homologue, amino acid similarity may be considered in addition to identity of amino acids at corresponding positions in an amino acid sequence. “Amino acid similarity” refers to amino acid identity and conservative amino acid substitutions in a putative homologue compared to the corresponding amino acid positions in a reference protein.

Conservative amino acid substitutions can be made in reference proteins to produce variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

With regard to nucleic acids, it will be appreciated by those of skill in the art that due to the degenerate nature of the genetic code, multiple nucleic acid sequences can encode a particular protein, and that such alternate nucleic acids may be used in compositions and methods of the present invention.

Percent identity is determined by comparison of amino acid or nucleic acid sequences, including a reference amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). The two sequences compared are generally the same length or nearly the same length.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis. and publicly available from the National Center for Biotechnology Information.

A non-limiting example of a mathematical algorithm utilized for the comparison of two sequences is the algorithm of Karlin and Altschul, 1990, PNAS 87:2264-2268, modified as in Karlin and Altschul, 1993, PNAS. 90:5873-5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, word length=12 to obtain nucleotide sequences homologous to a nucleic acid molecules of the present invention. BLAST protein searches are performed with the XBLAST program parameters set, e.g., to score 50, word length=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389-3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more nucleic acid or amino acid mutations can be introduced without altering the functional properties of a given nucleic acid or protein, respectively.

Generation of a genetically modified immunodeficient mouse whose genome includes a mutation of the Rhbdf2 gene can be achieved by introduction of a gene targeting vector into a preimplantation embryo or stem cells, such as embryonic stem (ES) cells or induced pluripotent stem (iPS) cells.

The term “gene targeting vector” refers to a double-stranded recombinant DNA molecule effective to recombine with and mutate a specific chromosomal locus, such as by insertion into or replacement of the targeted gene.

For targeted gene mutation, a gene targeting vector is made using recombinant DNA techniques and includes 5′ and 3′ sequences which are homologous to the stem cell endogenous Rhbdf2 gene. The gene targeting vector optionally and preferably further includes a selectable marker such as neomycin phosphotransferase, hygromycin or puromycin. Those of ordinary skill in the art are capable of selecting sequences for inclusion in a gene targeting vector and using these with no more than routine experimentation. Gene targeting vectors can be generated recombinantly or synthetically using well-known methodology.

For methods of DNA injection of a gene targeting vector into a preimplantation embryo, the gene targeting vector is linearized before injection into non-human preimplantation embryos. Preferably, the gene targeting vector is injected into fertilized oocytes. Fertilized oocytes are collected from superovulated females the day after mating (0.5 dpc) and injected with the expression construct. The injected oocytes are either cultured overnight or transferred directly into oviducts of 0.5-day p.c. pseudopregnant females. Methods for superovulation, harvesting of oocytes, gene targeting vector injection and embryo transfer are known in the art and described in Manipulating the Mouse Embryo: A Laboratory Manual, 3rd edition, Cold Spring Harbor Laboratory Press; Dec. 15, 2002, ISBN-10: 0879695919. Offspring can be tested for the presence of Rhbdf2 gene mutation by DNA analysis, such as PCR, Southern blot or sequencing. Mice having a mutation of the Rhbdf2 gene can be tested for iRhom protein expression such as by using ELISA or Western blot analysis and/or mRNA expression such as by RT-PCR.

Alternatively the gene targeting vector may be transfected into stem cells (ES cells or iPS cells) using well-known methods, such as electroporation, calcium-phosphate precipitation and lipofection.

Mouse ES cells are grown in media optimized for the particular line. Typically ES media contains 15% fetal bovine serum (FBS) or synthetic or semi-synthetic equivalents, 2 mM glutamine, 1 mM Na Pyruvate, 0.1 mM non-essential amino acids, 50 U/ml penicillin and streptomycin, 0.1 mM 2-mercaptoethanol and 1000 U/ml LIF (plus, for some cell lines chemical inhibitors of differentiation) in Dulbecco's Modified Eagle Media (DMEM). A detailed description is known in the art (Tremml et al., 2008, Current Protocols in Stem Cell Biology, Chapter 1:Unit 1C.4). For review of inhibitors of ES cell differentiation, see Buehr, M., et al., 2003. Genesis of embryonic stem cells. Philosophical Transactions of the Royal Society B: Biological Sciences 358, 1397-1402.

The cells are screened for Rhbdf2 gene mutation by DNA analysis, such as PCR, Southern blot or sequencing. Cells with the correct homologous recombination event resulting in mutation of the Rhbdf2 gene can be tested for iRhom protein expression such as by using ELISA or Western blot analysis and/or mRNA expression such as by RT-PCR. If desired, the selectable marker can be removed by treating the stem cells with Cre recombinase. After Cre recombinase treatment the cells are analyzed for the presence of the nucleic acid encoding a mutant iRhom2.

Selected stem cells with the correct genomic event mutating the Rhbdf2 gene can be injected into preimplantation embryos. For microinjection, ES or iPS cell are rendered to single cells using a mixture of trypsin and EDTA, followed by resuspension in ES media. Groups of single cells are selected using a finely drawn-out glass needle (20-25 micrometer inside diameter) and introduced through the embryo's zona pellucida and into the blastocysts cavity (blastocoel) using an inverted microscope fitted with micromanipulators. Alternatively to blastocyst injection, stem cells can be injected into early stage embryos (e.g. 2-cell, 4-cell, 8-cell, premorula or morula). Injection may be assisted with a laser or piezo pulses drilled opening the zona pellucida. Approximately 9-10 selected stem cells (ES or iPS cells) are injected per blastocysts, or 8-cell stage embryo, 6-9 stem cells per 4-cell stage embryo, and about 6 stem cells per 2-cell stage embryo. Following stem cell introduction, embryos are allowed to recover for a few hours at 37° C. in 5% CO₂, 5% O₂ in nitrogen or cultured overnight before transfer into pseudopregnant recipient females. In a further alternative to stem cell injection, stem cells can be aggregated with morula stage embryos. All these methods are well established and can be used to produce stem cell chimeras. For a more detailed description see Manipulating the Mouse Embryo: A Laboratory Manual, 3rd edition, Nagy et al., Cold Spring Harbor Laboratory Press; Dec. 15, 2002, ISBN-10: 0879695919, 1990, Development 110, 815-821; U.S. Pat. No. 7,576,259; U.S. Pat. No. 7,659,442; U.S. Pat. No. 7,294,754; and Kraus et al., 2010, Genesis 48, 394-399.

Pseudopregnant embryo recipients are prepared using methods known in the art. Briefly, fertile female mice between 6-8 weeks of age are mated with vasectomized or sterile males to induce a hormonal state conductive to supporting surgically introduced embryos. At 2.5 days post coitum (dpc) up to 15 of the stem cell containing blastocysts are introduced into the uterine horn very near to the uterus-oviduct junction. For early stage embryos and morula, such embryos are either cultured in vitro into blastocysts or implanted into 0.5 dpc or 1.5 dpc pseudopregnant females according to the embryo stage into the oviduct. Chimeric pups from the implanted embryos are born 16-20 days after the transfer depending on the embryo age at implantation. Chimeric males are selected for breeding. Offspring can be analyzed for transmission of the ES cell genome by appearance of reduced hair, i.e. hairless phenotype, and/or nucleic acid analysis, such as PCR, Southern blot or sequencing. Further the expression of mutant iRhom can be assayed by detection of mRNA encoding mutant iRhom or mutant protein expression such as by protein analysis, e.g. immunoassay, or functional assays, to confirm Rhbdf2 gene mutation. Offspring having the Rhbdf2 gene mutation are intercrossed to create non-human animals homozygous for the Rhbdf2 gene mutation. The transgenic mice are crossed to the immunodeficient mice to create a congenic immunodeficient strain with the Rhbdf2 gene mutation.

Methods of assessing a genetically modified non-human animal to determine whether the Rhbdf2 gene is mutated such that the mouse expresses the mutated Rhbdf2 gene are well-known and include standard techniques such as nucleic acid assays, spectrometric assays, immunoassays and functional assays.

One or more standards can be used to allow quantitative determination of an iRhom in a sample.

Assays for assessment of function of mutant iRhom2 in a mouse having a putative mutant Rhbdf2 gene can be performed. Assays for assessment of function putative mutant iRhom2 in a mouse having a putative Rhbdf2 gene mutation include assessment of hair of the mouse.

Optionally, genetically modified immunodeficient mice of the present invention are produced by selective breeding. A first parental strain of mouse which has a first desired genotype may be bred with a second parental strain of mouse which has a second desired genotype to produce offspring which are genetically modified mice having the first and second desired genotypes. For example, a first mouse which is immunodeficient may be bred with a second mouse which has a Rhbdf2 gene mutation to produce offspring which are immunodeficient and have a Rhbdf2 gene mutation and express the mutant iRhom2 encoded by the Rhbdf2 gene mutation, resulting in a hairless phenotype and an increase in growth of xenogeneic tumors in the genetically modified immunodeficient mice including one or more Rhbdf2 gene mutations compared to mice of the same background without the one or more Rhbdf2 gene mutations.

In further examples, a NOD.Cg-Prkdc^{scid} Il2rg^{tm1Wjl}/SzJ (NSG) mouse, NOD.Cg-Prkdc^{scid} Il2rg^{tm1Sug}/JicTac (NOG) or a NOD.Cg-Rag1^{tm1Mom} Il2rg^{tm1Wjl}/SzJ (NRG) mouse may be bred with a mouse which has a Rhbdf2 gene mutation to produce offspring which are immunodeficient and have a Rhbdf2 gene mutation and express the mutant iRhom encoded by the Rhbdf2 gene mutation, resulting in a hairless phenotype and an increase in growth of xenogeneic tumors of the immunodeficient offspring carrying the mutation.

Aspects of the invention provide genetically modified immunodeficient mice that include a Rhbdf2 gene mutation in substantially all of their cells, as well as genetically modified immunodeficient mice that include a Rhbdf2 gene mutation in some, but not all their cells.

According to aspects of the present invention, xenogeneic tumor cells are administered to a genetically modified immunodeficient mouse of the present invention which has a mutated Rhbdf2 gene such that the mouse expresses a corresponding mutant iRhom, resulting in a hairless phenotype and an increase in growth of xenogeneic tumors from xenogeneic tumor cells administered to the mouse, providing a tumor model of proliferative disease. According to aspects of the present invention, xenogeneic tumor cells are administered to a genetically modified NSG, NOG or NRG mouse of the present invention which has a mutated Rhbdf2 gene such that the mouse expresses a corresponding mutant iRhom2, resulting in a hairless phenotype and an increase in growth of xenogeneic tumors from xenogeneic tumor cells administered to the mouse.

According to aspects of the present invention, xenogeneic tumor cells are administered to a NOD.Cg-Prkdc^{scid}Il2rg^{tm1Wjl}Rhbdf^{I156}/SzJ, NOD.Cg-Prkdc^{scid}Il2rg^{tm1Wjl}Rhbdf^{D158X}/SzJ or NOD.Cg-Prkdc^{scid}Il2rg^{tm1Wjl}Rhbdf^{P159X}/SzJ mouse, wherein the mouse has a hairless phenotype and an increase in growth of xenogeneic tumors compared to mice of the same background without the Rhbdf2 gene mutations.

According to aspects of the present invention, xenogeneic tumor cells are administered to a NOD.Cg-Prkdc^{scid}Il2rg^{tm1Wjl}Rhbdf^{I156}/SzJ, NOD.Cg-Prkdc^{scid}Il2rg^{tm1Wjl}Rhbdf^{D158X}/SzJ or NOD.Cg-Prkdc^{scid}Il2rg^{tm1Wjl}Rhbdf^{P159X}/SzJ mouse, wherein the mouse has a hairless phenotype and an increase in growth of xenogeneic tumors compared to mice of the same background without the Rhbdf2 gene mutations.

Xenogeneic tumor cells administered to genetically modified immunodeficient mice of the present invention can be any of various tumor cells, including but not limited to, cells of a tumor cell line and primary tumor cells. The xenogeneic tumor cells may be derived from any of various organisms, preferably mammalian, including human, non-human primate, rat, guinea pig, rabbit, cat, dog, horse, cow, goat, pig and sheep.

According to specific aspects of the present invention, the xenogeneic tumor cells are human tumor cells. According to specific aspects of the present invention, the human tumor cells are present in a sample obtained from the human, such as, but not limited to, in a blood sample, tissue sample, or sample obtained by biopsy of a human tumor.

Tumor cells obtained from a human can be administered directly to a genetically modified immunodeficient mouse of the present invention or may be cultured in vitro prior to administration to the mouse.

As used herein, the term “tumor” refers to cells characterized by unregulated growth including, but not limited to, pre-neoplastic hyperproliferation, cancer in-situ, neoplasms, metastases and solid and non-solid tumors. Examples of tumors are those caused by cancer include, but are not limited to, lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer.

Tumor cells can be administered by various routes, such as, but not limited to, by subcutaneous injection, intraperitoneal injection or injection into the tail vein.

Engraftment of xenogeneic tumor cells can be assessed by any of various methods, such as, but not limited to, visual inspection of the mouse for signs of tumor formation.

The number of tumor cells administered is not considered limiting. A single tumor cell can expand into a detectable tumor in the genetically modified immunodeficient animals described herein. The number of administered tumor cells is generally in the range of 1000-1×10⁶ tumor cells, although more or fewer can be administered.

An increase in growth of xenogeneic tumors in a genetically modified immunodeficient mouse having a mutation of the Rhbdf2 gene of the present invention, wherein the immunodeficient mouse expresses a corresponding iRhom2 with a mutation in the N-terminal region, can be observed compared to an immunodeficient mouse of the same background without the Rhbdf2 gene mutation. The increase can be any detectable increase, such as, an increase in tumor volume of 1% or more compared to an immunodeficient mouse of the same background without the Rhbdf2 gene mutation over the same time period, such as 10% or more compared to an immunodeficient mouse of the same background without the Rhbdf2 gene mutation over the same time period, such as 20%, 30%, 40%, 50%, 60%, 70%, 80%, 90%, 100%, 200%, 300%, 400%, 500%, 600%, 700%, 800%, 900%, 1000% or greater increase in tumor volume compared to an immunodeficient mouse of the same background without the Rhbdf2 gene mutation over the same time period.

Any of various methods can be used to measure growth of xenogeneic tumors, including but not limited to, measurement in living mice, measurement of tumors excised from living mice or measurement of tumors in situ or excised from dead mice. Measurements can be obtained using a measuring instrument such as a caliper, measurement using one or more imaging techniques such as ultrasonography, computed tomography, positron emission tomography, fluorescence imaging, bioluminescence imaging, magnetic resonance imaging and combinations of any two or more of these or other tumor measurement methods. The number of tumor cells in a sample obtained from a mouse bearing xenogeneic tumor cells can be used to measure tumor growth, particularly for non-solid tumors. For example, the number of non-solid tumor cells in a blood sample can be assessed to obtain a measurement of growth of a non-solid tumor in a mouse.

Methods for identifying anti-tumor activity of a composition according to aspects of the present invention include providing a genetically modified immunodeficient mouse having a mutated Rhbdf2 gene such that the genetically modified immunodeficient mouse expresses a corresponding mutant iRhom, has a hairless phenotype and an increase in growth of xenogeneic tumors compared to mice of the same background without the Rhbdf2 gene mutations; administering xenogeneic tumor cells to the genetically modified immunodeficient mouse, wherein the xenogeneic tumor cells form a solid or non-solid tumor in the genetically modified immunodeficient mouse; administering a test compound to the genetically modified immunodeficient mouse; assaying a response of the xenogeneic tumor and/or tumor cells to the test compound, wherein an inhibitory effect of the test substance on the tumor and/or tumor cells identifies the test substance as having anti-tumor activity.

Methods for identifying anti-tumor activity of a composition according to aspects of the present invention include providing a genetically modified NSG, NOG or NRG mouse including a mutated Rhbdf2 gene such that the genetically modified immunodeficient mouse expresses a corresponding mutant iRhom has a hairless phenotype and an increase in growth of xenogeneic tumors compared to mice of the same background without the Rhbdf2 gene mutation; administering xenogeneic tumor cells to the genetically modified immunodeficient mouse, wherein the xenogeneic tumor cells form a tumor in the genetically modified immunodeficient mouse; administering a test compound to the genetically modified immunodeficient mouse; assaying a response of the xenogeneic tumor and/or tumor cells to the test compound, wherein an inhibitory effect of the test substance on the tumor and/or tumor cells identifies the test substance as having anti-tumor activity.

Methods for identifying anti-tumor activity of a composition according to aspects of the present invention include providing a genetically modified NSG mouse including a mutation in the Rhbdf2 gene such that the genetically modified NSG mouse expresses a corresponding mutant iRhom having a mutation in the N-terminal region, wherein the mouse has a hairless phenotype and an increase in growth of xenogeneic tumors compared to mice of the same background without the Rhbdf2 gene mutation; administering xenogeneic tumor cells to the genetically modified NSG mouse, wherein the xenogeneic tumor cells form a tumor in the genetically modified NSG mouse; administering a test compound to the genetically modified NSG mouse; assaying a response of the xenogeneic tumor and/or tumor cells to the test compound, wherein an inhibitory effect of the test substance on the tumor and/or tumor cells identifies the test substance as having anti-tumor activity.

Methods for identifying anti-tumor activity of a composition according to aspects of the present invention include providing a NOD.Cg-Prkdc^{scid}Il2rg^{tm1Wjl}Rhbdf^{I156}/SzJ, NOD.Cg-Prkdc^{scid}Il2rg^{tm1Wjl}Rhbdf^{D158X}/SzJ or NOD.Cg-Prkdc^{scid}Il2rg^{tm1Wjl}Rhbdf^{P159X}/SzJ mouse which has a hairless phenotype and an increase in growth of xenogeneic tumors compared to mice of the same background without the Rhbdf2 gene mutation; administering xenogeneic tumor cells to the mouse, wherein the xenogeneic tumor cells form a tumor in the mouse; administering a test compound to the mouse; assaying a response of the xenogeneic tumor and/or tumor cells to the test compound, wherein an inhibitory effect of the test substance on the tumor and/or tumor cells identifies the test substance as an anti-tumor composition.

Methods for identifying anti-tumor activity of a composition according to aspects of the present invention include providing a NOD.Cg-Prkdc^{scid}Il2rg^{tm1Wjl}Rhbdf^{I156}/SzJ, NOD.Cg-Prkdc^{scid}Il2rg^{tm1Wjl}Rhbdf^{D158X}/SzJ or NOD.Cg-Prkdc^{scid}Il2rg^{tm1Wjl}Rhbdf^{P159X}/SzJ which has a hairless phenotype and an increase in growth of xenogeneic tumors compared to mice of the same background without the Rhbdf2 gene mutation; administering xenogeneic tumor cells to the mouse, wherein the xenogeneic tumor cells form a tumor in the mouse; administering a test compound to the mouse; assaying a response of the xenogeneic tumor and/or tumor cells to the test compound, wherein an inhibitory effect of the test substance on the tumor and/or tumor cells identifies the test substance as having anti-tumor activity.

Assaying a response of the xenogeneic tumor and/or tumor cells to the test compound includes comparing the response to a standard to determine the effect of the test substance on the xenogeneic tumor cells according to aspects of methods of the present invention, wherein an inhibitory effect of the test substance on the xenogeneic tumor cells identifies the test substance as an anti-tumor composition. Standards are well-known in the art and the standard used can be any appropriate standard. In one example, a standard is a compound known to have an anti-tumor effect. In a further example, non-treatment of a comparable xenogeneic tumor provides a base level indication of the tumor growth without treatment for comparison of the effect of a test substance. A standard may be a reference level of expected tumor growth previously determined in an individual comparable mouse or in a population of comparable mice and stored in a print or electronic medium for recall and comparison to an assay result.

Assay results can be analyzed using statistical analysis by any of various methods to determine whether the test substance has an inhibitory effect on a tumor, exemplified by parametric or non-parametric tests, analysis of variance, analysis of covariance, logistic regression for multivariate analysis, Fisher's exact test, the chi-square test, Student's T-test, the Mann-Whitney test, Wilcoxon signed ranks test, McNemar test, Friedman test and Page's L trend test. These and other statistical tests are well-known in the art as detailed in Hicks, C M, Research Methods for Clinical Therapists: Applied Project Design and Analysis, Churchill Livingstone (publisher); 5th Ed., 2009; and Freund, R J et al., Statistical Methods, Academic Press; 3rd Ed., 2010.

The term “inhibitory effect” as used herein refers to an effect of the test substance to inhibit one or more of: tumor growth, tumor cell metabolism and tumor cell division.

A test substance used in a method of the present invention can be any chemical entity, illustratively including a synthetic or naturally occurring compound or a combination of a synthetic or naturally occurring compound, a small organic or inorganic molecule, a protein, a peptide, a nucleic acid, a carbohydrate, an oligosaccharide, a lipid or a combination of any of these.

According to aspects of the present invention, a test substance is an anti-cancer agent.

Anti-cancer agents are described, for example, in Brunton et al. (eds.), Goodman and Gilman's The Pharmacological Basis of Therapeutics, 12th Ed., Macmillan Publishing Co., 2011.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, cobimetinib, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, vemurafenib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

According to aspects of the present invention, an anti-cancer agent is an anti-cancer antibody. An anti-cancer antibody used can be any antibody effective to inhibit at least one type of tumor, particularly a human tumor. Anti-cancer antibodies include, but are not limited to, 3F8, 8H9, abagovomab, abituzumab, adalimumab, adecatumumab, aducanumab, afutuzumab, alacizumab pegol, alemtuzumab, amatuximab, anatumomab mafenatox, anetumab ravtansine, apolizumab, arcitumomab, ascrinvacumab, atezolizumab, bavituximab, belimumab, bevacizumab, bivatuzumab mertansine, brentuximab vedotin, brontictuzumab, cantuzumab mertansine, cantuzumab ravtansine, capromab pendetide, catumaxomab, cetuximab, citatuzumab bogatox, cixutumumab, clivatuzumab tetraxetan, coltuximab ravtansine, conatumumab, dacetuzumab, dalotuzumab, demcizumab, denintuzumab mafodotin, depatuxizumab mafodotin, durvalumab, dusigitumab, edrecolomab, elotuzumab, emactuzumab, emibetuzumab, enoblituzumab, enfortumab vedotin, enavatuzumab, epratuzumab, ertumaxomab, etaracizumab, farletuzumab, ficlatuzumab, figitumumab, flanvotumab, futuximab, galiximab, ganitumab, gemtuzumab, girentuximab, glembatumumab vedotin, ibritumomab tiuxetan, igovomab, imab362, imalumab, imgatuzumab, indatuximab ravtansine, indusatumab vedotin, inebilizumab, inotuzumab ozogamicin, intetumumab, ipilimumab, iratumumab, isatuximab, labetuzumab, lexatumumab, lifastuzumab vedotin, lintuzumab, lirilumab, lorvotuzumab mertansine, lucatumumab, lumiliximab, lumretuzumab, mapatumumab, margetuximab, matuzumab, milatuzumab, mirvetuximab soravtansine, mitumomab, mogamulizumab, moxetumomab pasudotox, nacolomab tafenatox, naptumomab estafenatox, narnatumab, necitumumab, nesvacumab, nimotuzumab, nivolumab, ocaratuzumab, ofatumumab, olaratumab, onartuzumab, ontuxizumab, oregovomab, oportuzumab monatox, otlertuzumab, panitumumab, pankomab, parsatuzumab, patritumab, pembrolizumab, pemtumomab, pertuzumab, pidilizumab, pinatuzumab vedotin, polatuzumab vedotin, pritumumab, racotumomab, radretumab, ramucirumab, rilotumumab, rituximab, robatumumab, sacituzumab govitecan, samalizumab, seribantumab, sibrotuzumab, siltuximab, sofituzumab vedotin, tacatuzumab tetraxetan, tarextumab, tenatumomab, teprotumumab, tetulomab, tigatuzumab, tositumomab, tovetumab, trastuzumab, tremelimumab, tucotuzumab celmoleukin, ublituximab, utomilumab, vandortuzumab vedotin, vantictumab, vanucizumab, varlilumab, vesencumab, volociximab, vorsetuzumab mafodotin, votumumab, zalutumumab and zatuximab.

Embodiments of inventive compositions and methods are illustrated in the following examples. These examples are provided for illustrative purposes and are not considered limitations on the scope of inventive compositions and methods.

### EXAMPLES

### Example 1

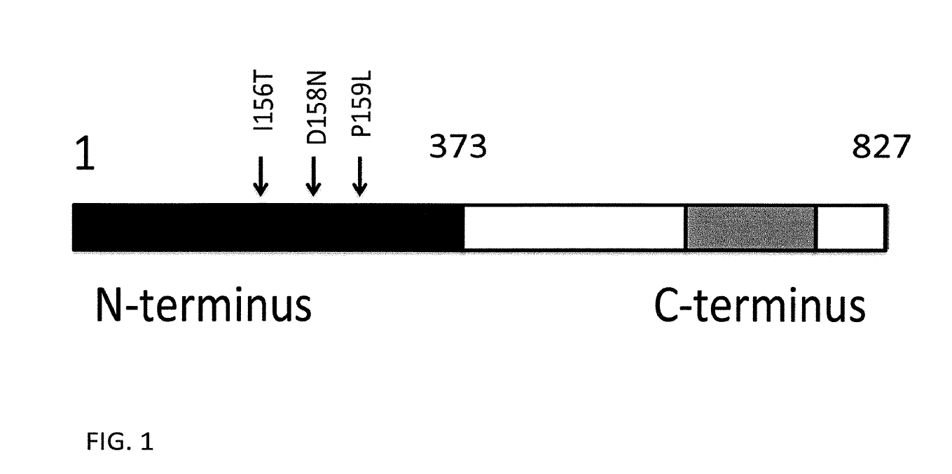

NSG-BALDP159L mice carrying a point mutation p.P159L in the Rhbdf2 gene were generated using CRISPR/Cas9 technology. The Rhbdf2 locus in NSG embryos was targeted by pronuclear microinjection of Cas9 mRNA, truncated guide RNA (sgRNA) and single stranded oligonucleotide DNA (ssDNA; CGTGCAAGATGCCCAAGGTGGGCCCCCTGGAGGTGATGGGCAGCAAGCGGC TCTCCCAGGGTCTGGGCAACATTGTTCACCCACATCTCTTGCAGATTGTGGAT CTACTGGCTCGGGGTAGGGCCTTCCGCCATCCAGATGAGGTGGACCGGCCTC ACGCTGCCCACCCACCTCTGACTCCAGGGGTCCTGTCTCTCAC (SEQ ID NO:4) to mutate the proline at amino acid 159 of SEQ ID NO:1 to leucine. The underlined codon CTA is the mutant codon being introduced into the genome. A schematic diagram of mouse iRhom2 (SEQ ID NO:1) is shown in FIG. 1.

sgRNA used to generate the mice (17mer): G CAG ATT GTG GAT CCA C (SEQ ID NO:7)

CRISPR/Cas9 Protocol:
1. Turn on PCR machine, and the centrifuge—close lid, set temp to 4° C.
2. RNase-Zap your work area.
3. Remove to ice from −80° C. (in labeled box right in the front, bottom shelf)
   a. sgRNA
   b. Cas9 mRNA
4. Spin guides and Cas9mRNA at 20,000×g for 15-30 min, at 4° C.
5. Removing from the top of each sample, make up dilutions of the sgRNA's and Cas9—
   a. 4 ul Tris/EDTA (TE)+4 ul guide.
      i. Mix well, then Biospek 2 ul (program=RNA 1 mm)
      ii. Calculate what volume of IDTE to add to the remaining 6 ul to bring the sgRNA's to a final concentration of 300 ng/ul, and the Cas9mRNA to a final concentration of 500 ng/ul.
      iii. After adding TE to dilute, mix well (vortex, quick spin).
6. Combine in a PCR tube:
   a. 16.25 ul of TE
   b. 3.00 ul of Cas9mRNA
   c. 1.25 ul sgRNA
   d. Mix, quick spin, place into PCR machine and perform “renature” program (under RNA folder)
7. Dilute Plasmid to concentration 125 ng/ul
   a. 2 ul TE+5 ul plasmid
      i. Mix well, then Biospek 2 ul (program=DNA_1 mm)
      ii. Calculate what volume of TE to add to the remaining 4 ul to bring this to a final concentration of 125 ng/ul.
      iii. After adding IDTE, mix well (vortex, quick spin).
8. Dilute RNASin 10-fold (1 ul in 9 ul IDTE)
9. After renaturation is complete, remove sample to ice. Once cooled, the sample is vortexed to mix and quick spin.
10. Finally, add the diluted plasmid (2 ul) and RNASin (1.25 ul). The volume should now be 25 ul. Mix and quick spin, then transfer 20 ul to a new tube. For microinjection into mouse zygotes, first inject the DNA/RNA material into the pronucleus and then into the cytoplasm upon withdrawal of the needle.

A total of 16 pups were born, and surprisingly, 8 of 16 pups exhibited a partial (95% or more) to complete (100%) hair loss. DNA obtained from tail cells of the pups was amplified using PCR, cleaned-up, sequenced to identify and confirm which of the pups carried the p.P159L mutation.

The following sequencing primers are used to identify the point mutation

Forward:
(SEQ ID NO: 8)
ACACACACATGTACCGCCAT
Reverse:
(SEQ ID NO: 9)
TTCTGGCCTTTAGGGTGTGC

PCR cycling conditions are shown in Table I:

TABLE I

| Step | Temperature (° C.) | Time | |
|---|---|---|---|
| 1 | 95 | 30 | sec |
| 2 | 95 | 15 | sec |
| 3 | 62.5 | 30 | sec |
| 4 | 68 | 1:00 | min |
| 5 | Go to step 2 | 35 | cycles |

| | | |
|---|---|---|
| 6 | 68 | 5:00 |
| 7 | 10 | hold |

Magnetic Bead Cleanup Protocol for PCR Products

Typical: 15 ul PCR+3 ul 6× dye->run 8 ul on gel, purify remaining 10 ul
1. Add 18 ul of well-mixed beads (ratio=1.8 ul beads per 1 ul PCR product).
2. Mix well, then quick spin (keep as short as possible, just bring liquid down, don't want to pellet/clump beads).
3. Let sit at RT for at least 5 minutes.
4. Place on magnet, let sit for at least 2 minutes.
5. Wash by adding 100 ul of 70% Ethanol to each sample. Invert onto paper towels. Repeat at least two more times, or until all evidence of the loading dye is gone.
6. A small amount of ethanol will remain at the bottom. After the last wash, use a gel loading pipet tip with filter to remove as much of that as possible while leaving the beads intact.
7. Allow ethanol to evaporate off of the samples while still on the magnet, typically ˜10 minutes.
8. Remove from magnet, check for complete evaporation of ethanol, then add 40 ul of water. Mix thoroughly followed by a quick spin (again, as short as possible).
9. Place samples back on the magnet and let sit for at least 2 minutes.
10. Using the gel loading pipet to avoid contact with the beads, remove 25 ul (or more, if you can do so without bringing beads with the sample) to a new tube.
11. The purified sample can now be used for sequencing (5 ul of purified sample+1 ul of 5 uM sequencing primers). If desired, samples can be run out on a gel to confirm cleanup was successful.

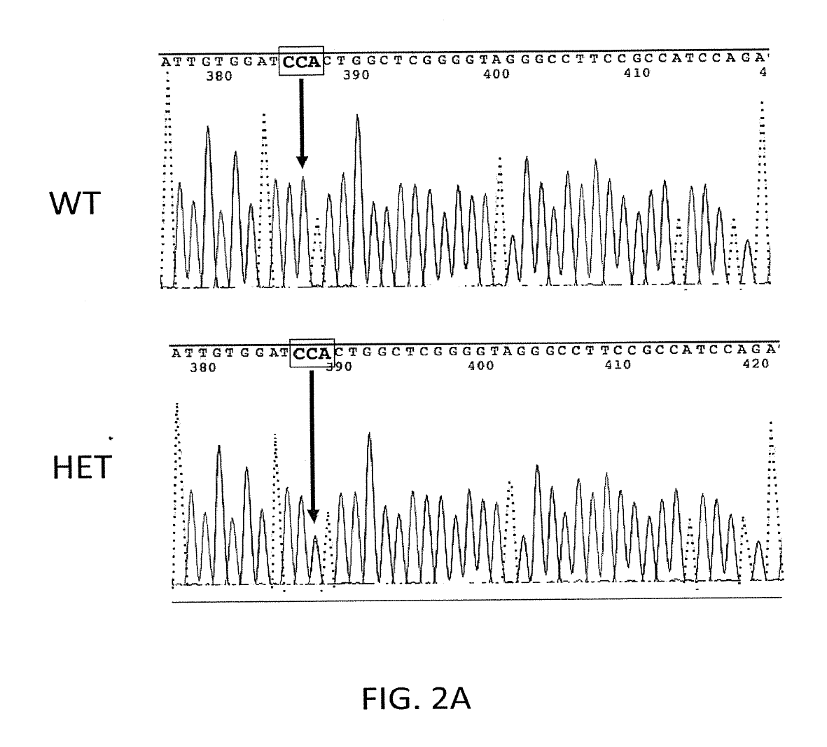

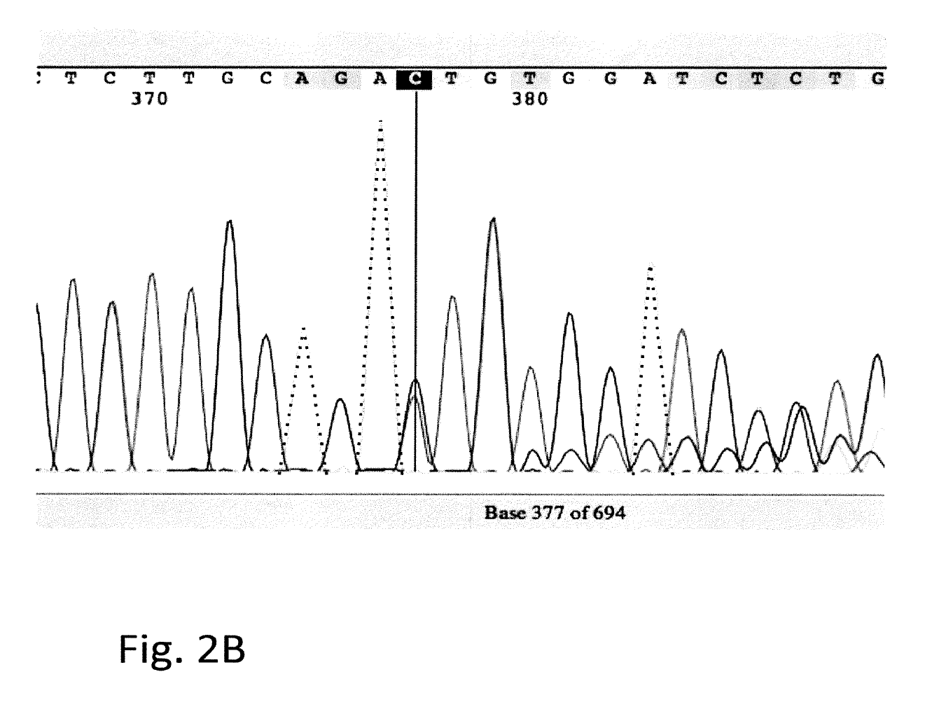

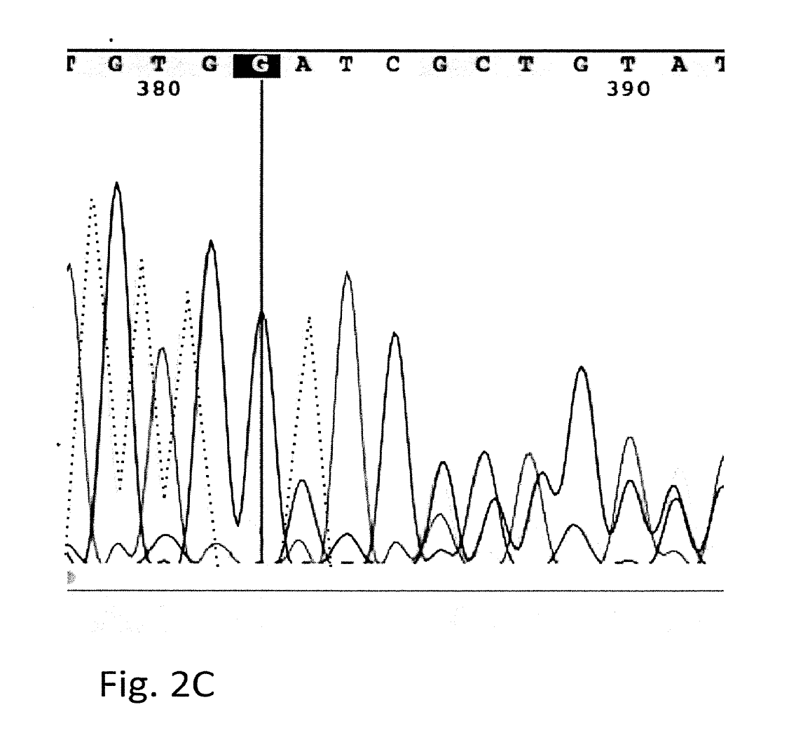

Results of sequencing distinguish a wild-type mouse from a mutated mouse heterozygous for the p.P159L mutation as shown in FIG. 2. Founder mice carrying the p.P159L mutation were backcrossed (N1) to NSG mice to check generate mutant mice by germline transmission. The resulting offspring (N1F1) heterozygous for the Rhbdf2^{P159L} allele were intercrossed to generate homozygous Rhbdf2^{P159L} mice. Mice homozygous for the Rhbdf2^{P159L} allele are characterized by a hairless phenotype. Mice homozygous for the Rhbdf2^{D158N} allele are characterized by a hairless phenotype. Mice homozygous for the Rhbdf2^{I156T} allele are characterized by a hairless phenotype. Mice homozygous for any two or more of: Rhbdf2^{I156T}, Rhbdf2^{D158N} and Rhbdf2^{P159L} allele are characterized by a hairless phenotype.

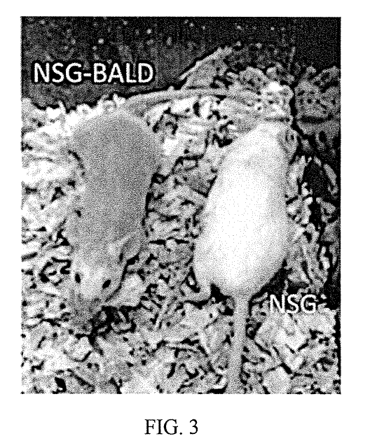

Loss of hair is clearly visible in the NSG-Bald mice and occurs by age 6 days. FIG. 3 shows a 6-week-old mouse carrying the Rhbdf2 p.P159L mutation (NSG-Bald) and characterized by a hairless phenotype (left) and a normal white haired littermate control mouse (right) carrying a wildtype Rhbdf2 allele.

A similar procedure is used to produce additional genetically engineered NSG mice with mutant iRhom2 including mutation at isoleucine 156 of SEQ ID NO:1, mutation at aspartic acid 158 of SEQ ID NO:1 and combinations of two or three mutations at 156, 158 and 159 using alternate oligonucleotides, producing genetically modified NSG mice, wherein the genome of the mice includes a mutated Rhbdf2 gene, the mice express mutant iRhom2 protein and are characterized by a hairless phenotype as shown in Table II.

TABLE II

| Mutation | Phenotype |
|---|---|
| p.I156T | hairless |
| p.D158N | hairless |
| p.P159L | hairless |
| p.I156T and/or p.D158N | more hairless than any of p.I156T, |
| and/or p.P159L | p.D158N or p.P159L alone |

Xenogeneic Tumor Cell Engraftment in NSG-BALD Mice

SKOV3 human ovarian carcinoma cells are obtained from ATCC (HTB-77) and cultured in RPMI medium. Six to eight week old NSG-BALD and NSG female mice (n=10) are injected with 0.5×10⁶ cells, suspended in 200 microliters of PBS, intraperitoneally. Body weight and tumor growth are monitored for 50 days, and at the end of the study period, necropsies are performed, tumor samples are fixed in 10% NBF, and paraffin-embedded for H&E staining.

Statistical Analysis

Kaplan-Meier survival curves are generated using GraphPad Prism. P value less than 0.05 is considered significant.

### Example 2

Animals

Six- to eight-week-old NSG-BALDP159L (n=5) and NSG female mice (n=4) were used to examine tumor growth. Mice were bred and maintained under specific pathogen free (SPF) conditions at The Jackson Laboratory. Food and acidified water were provided ad libitum.

Xenogeneic Tumor Cell Preparation and Administration

MDA-MB 231 human breast cancer cells were cultured in RPMI medium and grown at 37° C. MDA-MB 231 human breast cancer cells (3×10⁶) suspended in 200 μls of PBS were injected subcutaneously into NSG-BALDP159L and NSG mice.

Tumor Size

Subcutaneous xenograft tumor diameter was measured everyday using an external caliper. In order to determine tumor volume by external caliper, the greatest longitudinal diameter (length) and the greatest transverse diameter (width) were determined using the external caliper. Tumor volume was then calculated as: (length×width²)=tumor volume. At the end of the study, tumors were harvested and subjected to histological analysis.

Tumor Growth

Tumor growth was calculated using the formula: [tumor volume on a given day/tumor volume recorded on day 7]×100=percent increase in tumor volume.

Statistics

Data is represented as mean±standard error of the mean (SEM), and Student's t-test was used to calculate the differences in growth between NSG and NSG-BALDP159L mice. p-value less than 0.05 was considered to be significant. A significant difference in tumor growth was observed as early as day 18.

Results

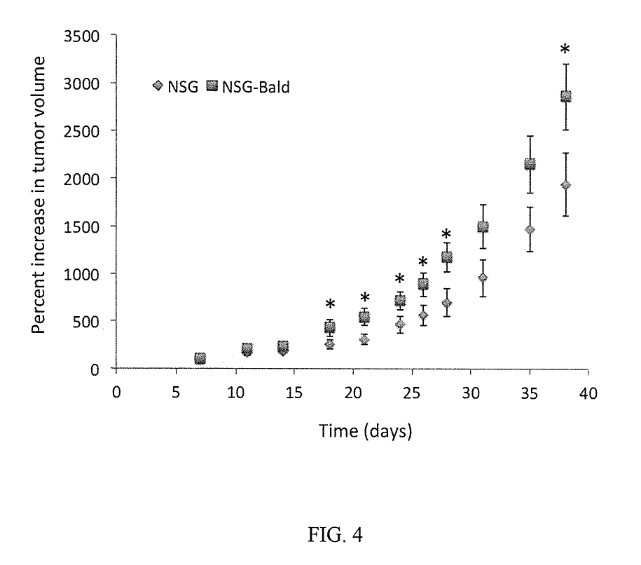

FIG. 4 is a plot showing the growth human breast tumor cell line MDA-MB 231 implanted into NSG mice (bottom line) and NOD.Cg-Prkdc^{scid}Il2rg^{tm1Wjl}Rhbdf^{P159L}/SzJ (NSG-BALDP159L) (top line) animals. The growth of the MDA-MB 231 human breast cancer cell tumors in NSG-BALDP159L (top line) mice is significantly faster compared with the control NSG mice, with an approximately two-fold increase in tumor volume observed in NSG-BALDP159L vs NSG mice.

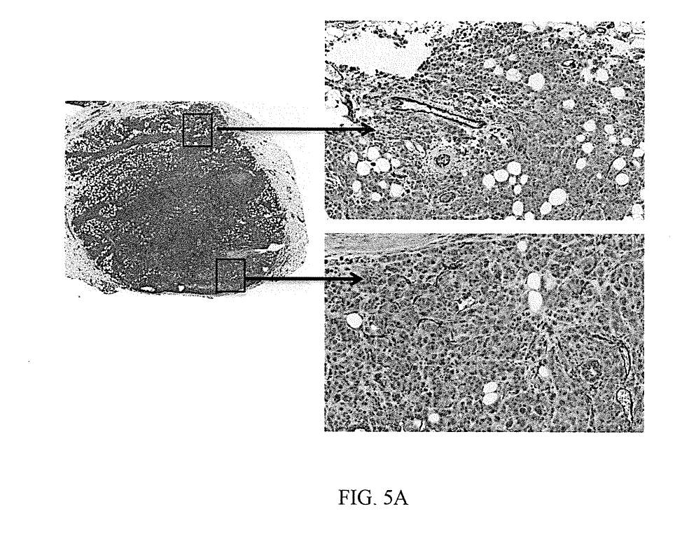

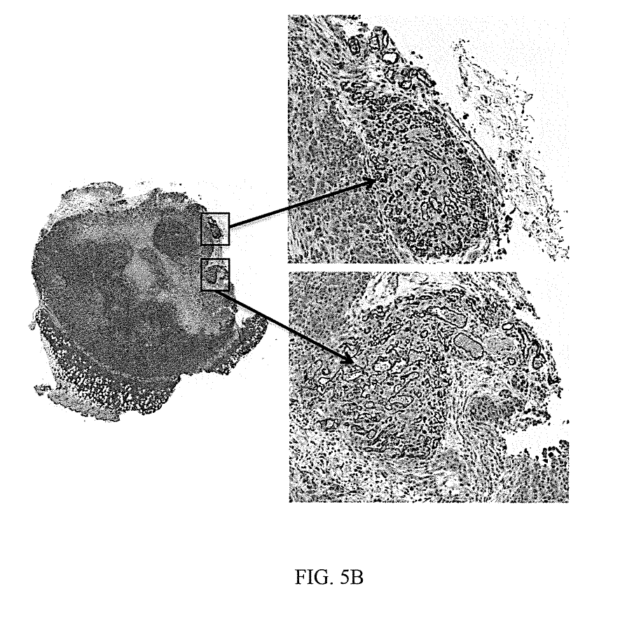

Immunostaining of MDA-MB 231 human breast cancer cell derived tumors for vascular marker CD31 in NSG (FIG. 5A) and NSG-BALDP159L (FIG. 5B) mice. Strong CD31 immunohistochemical staining is observed in xenogeneic tumors obtained from NSG-BALDP159L mice compared with xenogeneic tumors from NSG mice, indicating an increase in vascularization in xenogeneic tumors in the NSG-BALDP159L mice. Without being limited by theoretical considerations, it is appreciated that an increase in vascularization in xenogeneic tumors may support the increased growth of the tumors in a genetically engineered immunodeficient mouse according to aspects of the present invention expressing an iRhom2 protein with one or more mutations selected from the group consisting of: p.I156T, p.D158N and p.P159L.

### Example 3

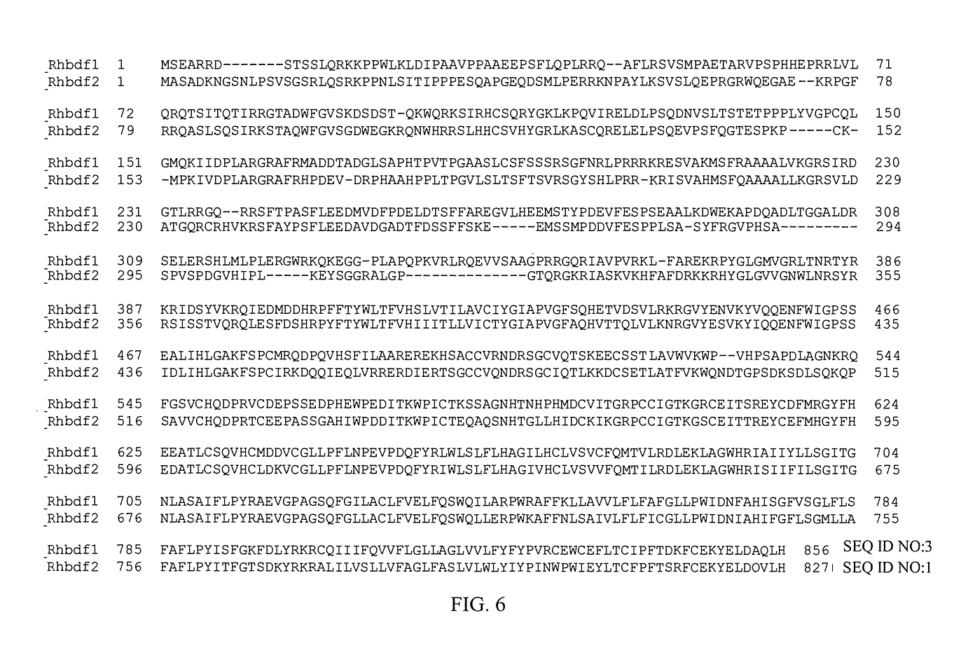

Mouse iRhom1 and mouse iRhom2 are highly related proteins as shown in FIG. 6 which is an alignment of SEQ ID NOs: 1 and 3. In view of the high degree of structural identity of mouse iRhom1 and mouse iRhom2, studies were performed to determine whether the functional effect of modifications in iRhom1 are analogous to those observed with modification of iRhom2.

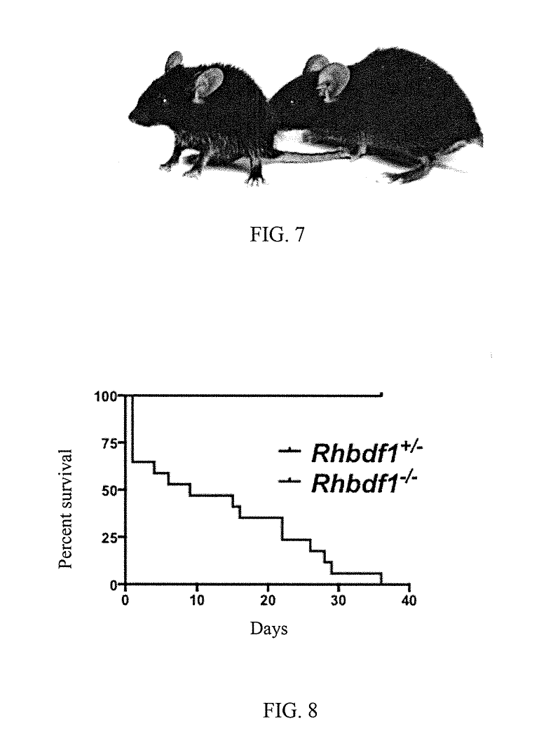

Rhbdf1 knockout C57BL/6 mice were generated, producing iRhom1 deficient mice and it was found that iRhom1 deficiency leads to weight loss. FIG. 7 demonstrates size differences between mice heterozygous for Rhbdf1 gene deletion (Rhbdf1^{+/-}) which are normal in size (right) compared to mice homozygous for Rhbdf1 gene deletion (Rhbdf1^{-/-}) which are smaller (left) due to weight loss;

Rhbdf1 knockout C57BL/6 mice die by 3-4 weeks of age as shown graphically in FIG. 8. Mice heterozygous for the Rhbdf1 gene deletion (Rhbdf1^{+/-}) display normal percent survival, top line of the graph in FIG. 8, and are viable and fertile, while mice homozygous for Rhbdf1 gene deletion (Rhbdf1^{-/-}) die by 3-4 weeks of age, bottom line, of the graph in FIG. 8;

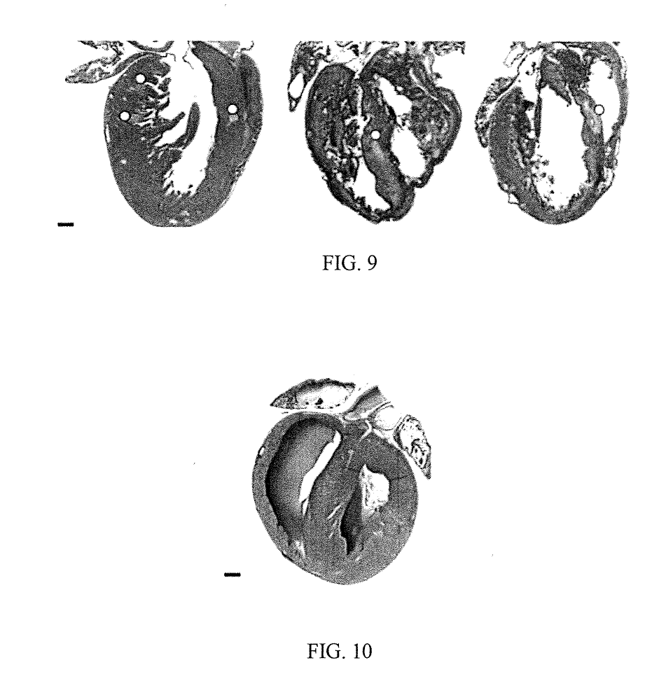

Rhbdf1 knockout C57BL/6 mice are characterized by severe cardiac fibrosis. Hearts of Rhbdf1 knockout C57BL/6 mice sectioned and stained hematoxylin and eosin show severe cardiac fibrosis, FIG. 9 marked with “∘”, which leads to death of these animals at around 3-4 weeks of age. In contrast, Rhbdf1^{-/+} heterozygous mice show no cardiac fibrosis, as exemplified in the image of a hematoxylin and eosin stained section of a heart isolated from a Rhbdf1^{-/+} heterozygous mouse, shown in FIG. 10.

Furthermore, it is not possible to grow tumors in these mice as Rhbdf1 knockout mice die shortly after birth. Still further, Rhbdf1 knockout C57BL/6 mice displayed no hairless phenotype, having a full coat of hair. Thus, surprisingly, in spite of the high degree of relatedness of mouse iRhom1 and mouse iRhom2, phenotypes associated with changes in these proteins are dissimilar.

Items

Item 1. A genetically modified immunodeficient mouse, wherein the genome of the mouse comprises a mutated Rhbdf2 gene such that the mouse expresses mutant iRhom2 protein which differs from wild-type mouse iRhom2 protein due to one or more mutations in the N-terminal region, and wherein the mouse is characterized by a hairless phenotype.

Item 2. The genetically modified immunodeficient mouse of item 1, wherein the mouse has severe combined immunodeficiency.

Item 3. The genetically modified immunodeficient mouse of item 1, wherein the mouse has an IL2 receptor gamma chain deficiency.

Item 4. The genetically modified immunodeficient mouse of any of items 1-3, wherein the mouse comprises the scid mutation.

Item 5. The genetically modified immunodeficient mouse of any of items 1-4, wherein the mouse is homozygous for the scid mutation.

Item 6. The genetically modified immunodeficient mouse of item 1, wherein the genetically modified immunodeficient mouse is a NOD.Cg-Prkdc^{scid}Il2rg^{tm1Wjl}/SzJ mouse comprising a mutated Rhbdf2 gene such that the mouse expresses mutant iRhom2 protein which differs from wild-type mouse iRhom2 protein due to one or more mutations in the N-terminal region, and wherein the mouse is characterized by a hairless phenotype.

Item 7. The genetically modified immunodeficient mouse of any of items 1-6, further comprising xenogeneic tumor cells.

Item 8. The genetically modified immunodeficient mouse of any of items 1-7, further comprising human tumor cells.

Item 9. The genetically modified immunodeficient mouse of any of items 1-8, wherein the one or more mutations in the N-terminal region is one or more mutations in the N-terminal region of iRhom2 selected from the group consisting of: substitution at amino acid 156, 158, 159 or two or more thereof.

Item 10. The genetically modified immunodeficient mouse of any of items 1-9, wherein the one or more mutations in the N-terminal region is one or more mutations in the N-terminal region of iRhom2 selected from the group consisting of: deletion of amino acid 156, 158, 159 or two or more thereof. Item 11. The genetically modified immunodeficient mouse of any of items 1-10, wherein the one or more mutations in the N-terminal region is deletion of all or part of the N-terminal region of iRhom2.

Item 12. The genetically modified immunodeficient mouse of any of items 1-11, wherein the mouse is homozygous for the mutation or deletion in the Rhbdf2 gene.

Item 13. The genetically modified immunodeficient mouse of any of items 1-12, wherein amino acids 374-827 of the mutant iRhom2 are identical to or substantially similar to the wild-type iRhom2 protein.

Item 14. The genetically modified immunodeficient mouse of any of items 1-12 having a mutation or deletion in the N-terminal region of the Rhbdf2 gene and expresses the corresponding mutant iRhom2, wherein the mouse is characterized by increased growth of an exogenous tumor compared to a mouse of the same genetic background which expresses the corresponding wild-type iRhom2 protein.

Item 15. A method for producing a mouse model system for response of xenogeneic tumor cells, comprising: providing a genetically modified immunodeficient mouse according to any of the preceding items; and administering xenogeneic tumor cells to the genetically modified immunodeficient mouse.

Item 16. A method for identifying an anti-tumor composition, comprising: providing a genetically modified immunodeficient mouse according to any of the preceding items; administering xenogeneic tumor cells to the genetically modified immunodeficient mouse; administering a test substance to the mouse; assaying a response of the xenogeneic tumor cells; and comparing the response to a standard to determine the effect of the test substance on the xenogeneic tumor cells, wherein an inhibitory effect of the test substance on the xenogeneic tumor cells identifies the test substance as an anti-tumor composition.

Sequences

Mouse iRhom2 protein sequence:
SEQ ID NO: 1
MASADKNGSNLPSVSGSRLQSRKPPNLSITIPPPESQAPGEQDSMLPERRKNPAYL
KSVSLQEPRGRWQEGAEKRPGFRRQASLSQSIRKSTAQWFGVSGDWEGKRQNW
HRRSLHHCSVHYGRLKASCQRELELPSQEVPSFQGTESPKPCKMPKIVDPLARGR
AFRHPDEVDRPHAAHPPLTPGVLSLTSFTSVRSGYSHLPRRKRISVAHMSFQAAA
ALLKGRSVLDATGQRCRHVKRSFAYPSFLEEDAVDGADTFDSSFFSKEEMSSMP
DDVFESPPLSASYFRGVPHSASPVSPDGVHIPLKEYSGGRALGPGTQRGKIMSK
VKHFAFDRKKRHYGLGVVGNWLNRSYRRSISSTVQRQLESFDSHRPYFTYWLTF
VHIIITLLVICTYGIAPVGFAQHVTTQLVLKNRGVYESVKYIQQENFWIGPSSIDLI
HLGAKFSPCIRKDQQIEQLVRRERDIERTSGCCVQNDRSGCIQTLIKKDCSETLATF
VKWQNDTGPSDKSDLSQKQPSAVVCHQDPRTCEEPASSGAHIWPDDITKWPICT
EQAQSNHTGLLHIDCKIKGRPCCIGTKGSCEITTREYCEFMHGYFHEDATLCSQV
HCLDKVCGLLPFLNPEVPDQFYRIWLSLFLHAGIVHCLVSVVFQMTILRDLEKLA
GWHRISIIFILS GITGNLASAIFLPYRAEVGPAGSQFGLLACLFVELFQSWQLLERP
WKAFFNLSAIVLFLFICGLLPWIDNIAHIFGFLSGMLLAFAFLPYITFGTSDKYRKR
ALILVSLLVFAGLFASLVLWLYIYPIWPWIEYLTCFPFTSRFCEKYELDQVLH
Mouse Rhbdf1 gene sequence: (bold indicates exons; underlined
indicates codons for amino acids 156, 158 and 159)
SEQ ID NO: 2
ACAGAAGCAGGCAGATCTCTGAGTTCAAGGATAGTCTGGTCTACAGGGCAAGTTCCAAGA
CTAGACAGAGAAACCCTGTATGGAAAAACAAACAAACAAACAAAAAAGTTGCAGGCCAGC
TGGGCGTGCTGGTTCACATCTTTAACCTCAGCTCCCAGGAGGCAGAGGCAGAGACAGGTG
AATCTCTGTGAATTTGAGACCAGTCTGGGCTAGATAGTAAATTCCAGGCCAGCCAAGGCT
ACATAGTGAAACTTTGTCTCAAGACAAGATAAGGGAATAAAAAAATATTCAAGCCAGTCT
GCTTTACAGAGCAAAGTACTGTCTCAATTTTTTCAGTATTTATTTCAATTTTTTACAGTT
TTTTTTAAGTTATAAAAACATAGCAAGTGTATGCATTCCACTTTTTGTTTTTCTACTCAA
GGGCATTTTGGAGATAATTCCCTATCAACACACAGTTACCTCATTGCTTTTAACAGCTGC
AATGTGACATTCCCACACGTTACACCCAACTGTTTCTCACTCTCCCACACCAGGCAGGAG
AGGTGGTTTACTGCAGCCAAGTCTGTGATCTCTCCATCTCCAGGGCTAGGGCGGCATGGC
ACAAGTCTCTGATGTCACCGAAGCATCATGCATATTCTCGTCCTTGGCCGTACAGGGGT
G
AGGACACAGCCCAGCCGTCCAGCAGTGAGGACACAGCCCAACCGTCCAGCAGTGAGGA
CA
CAGCCCAGCCCAGCCCAGCCCAGGTGCCAGAGTAGCATCTGCCAGGCTGAGAGGTGGA
TT
GGGCCTGAATGGTTCCAGCAGCCCCACGGTGCCTCTTCTGCCTGATGCTCTTTGCTGCC
A
TGGGACAGACAGAATGATCCTCACATGATGGAACTCACACGCATGCCAGGCAAGTCCAG
C
CTCCCACCTGCACCCCAACCCCCAGGGCTGGTTCTTTTGCCGCTCTCAGCATGGTTTCCT
AAGGATATGTGGAGGGGTCACTAAATGGCTCCCTGCCTGTGCTTCAGAGAAACAGCTCCC
AAGTTTGGGGTTATTATCTGTCTTTGCCCCAGCCTTGCCTTTCCGTGGCTGGAGACTGGG
AAGGAGAAGTTTGCCACCCTGCCTAATAGGAAGTAACTAAAGCTCTAAGCATGTGGTGCA
CACACATTTTATTTATTTATTTTTACATGAATTAGTGTTTTGCCTGTGTCATATATGTAT
GTATGTATGTATGTATGTATGTGTTAAGTATGTATTATGATGTACATGCCTGTGGAGGTA
TAAGAAGGGCATTTGATCTCTGAAACTGAGTTAAGGCTGTCTGCAAGATCCCAAGTGCAT
GCTGGGAACTGAACCTGGGTCTCTGCAACATCAGCAAAAGCTTCCAACCATATAATTATT
TCCCCTGCCCCACTTTTTTTTTTTTTTTTTTAAGATTTATTTATTGCCGGGCAGTGGTGG
CACACACCTTTAATCCCAGCACTTGGGAGGCAGGAGGATTTCTGAGTTGGAGGCCAGCCT
GGTCTACAGAGTGAGTTTCAGGACAGCCAGGGCTACACAGAGAAACACTATCTTGAAAAT
AAAAAAATAAAATAAATTTTAAAAATTCTTTTATTTATATGAGTCCACTATAGCTGTCAG
ACACACCAGAAGAGGGTATCAGATCCCATTACAGATGGTTGTGAGCCACCATGTGGTTGC
TGGGAGTTGAACTCAGGACCTCTGGAAGAGCAGTCAGTGCTTTTAACCTCTGCTCTACCC
AACCCACCAACGTGGCAGACTGGGGGCAGGGTGGTTAAGAGCAACAAGAGCCCAGAGACC
TGGCTCACCTCTGAAAGCAGCTCTGCTGCAGCGCCCCCTGGTGGTGGTCTCTCCATACTC
TCTGGCTGGGCGAGGACTTCAGAAAGAAAGACTGAGGCCATTGGTGCAGGGGCTGAGGAT
AGGGACTCCAGACCTGGGGGTACAGGTCTAGTTCGTTCCTCTGCCATTTCCTGCCTGCCG
TAAGTTTCCACATCAAATCCCAAGTGAGGGGCTAACCCAGGCCCTAGGCATCTGTATCAG
TGGCACCCCCTGCCCTTCTCCGGCCTGCTGTTCTTGTTCAGGAGCTGACAGGTCCGGCGA
GTGCTCGTAGGTGGGAGCATGGGAGTGTTGGACAGGGTGTCATAAATGTAGGCCTTCGTA
CAGGGCTAGGTACGCGAACATGAAGAGTGGTACTCTACCAGGAAGTGGGTAAGAACATCA
CAAGATGGCACCACCCAGAGCCGAGTTAAGGGAGGGATATCTGGGTCCAGGAGGGAGATG
AGGAGGCACAGCCCAGCTCCTATGGGCTCAAGGTGGCTAGAGACGTGGGCTAAGGAGGAT
AAAAGCCTGTGGCTTAAACTTGAGGGAGGGCCGGCCGTCACCACTACTAATAATAGCAAA
GATAACAGCAGCTGCTAGTTAGAGCCAGGGGCCCCACAAATGCTCCCTGTTACTGCTACC
ACACAGAGAGGGGAAAGCTGAGACCGAGGAGGCTTAGGGGATTCATCTAAGACCACAGGA
GCAGTCAATGGCAGATCAGGATTTGAACCCCCGGCTCTGTTAGCTGGAGTCATATATAGT
TATTTCTCATTACAGCCAACAAAGCAAGTTACTTGGTCAGTATTGGCCAGGCTAGAGTAT
CCAAAGCCTGGGCCTGGGGGCTGTTCATGACACGGAGATGTGGAGGGCCTTCCTCTGCAT
CTATTGCCAGTTACTGTGAGAGCCAGCTTTCAGCCTTAGCAAGAAGCCTCTGTGTCCTTG
GGTGCAGAGCAACATTCACAGTTTCCTAAGGGACAGTCCCAAGAACTAGCATATACCTCG
GTTGCCTTTCCAACTGCTCTGTGTTTAGTCCTGGGGTGGTTAAGGGGACAAGACCCAACT
TCCAGCAAGGACCCGGTCTGGCCTAGAAGGGATGCCAGGCCTGAGGAGAGATCATTCTAA
TGGACGAAGGAGAGACAGCAGCTAGAGAAGGCCAAGGCTTCCTAGACGATGTAGCTGCAG
CGATCGATCGGGGATTCTGGAAAGGATGCAAGCCTAGTCGAGGCTCCTGGAGTCAAAGGA
GCCTGAGGTCACACGAGAAAGAGAAGGGGAAATTGAGTGGTTTTTGCTTGGTTGTTATGG
GGGTGTGTCCGTGTGAGCGCGTGTGTTGCTGGAGATCGAACCTAAGATCTGTGTGCTAGG
CAAGTGTTTAAATGTGGCCATAAGTGGAGCAGCAGGAGCTACAAGCCCAGGCAGAAGCCG
CGGGCCGACCACGCCCCCCGAAGCACCGCCCACACACCAAGAAGCCCCGCCTCAATGAGG
CCCCGCCCACACCCAGTCCCCGCCCCTGCGCCCTCGCGCAGGTAGGGAAGAGGCGGAGCG
CTGGCGCTCAGCCTTGTAGCCGCCGCCCCGCCGCTGCCCACTCTGCTCTCAGCCGCTTCC
CGGGACGTGGGGCCTCCGAGAGGTGAGCACGGGGAATTGGGTGCGGCGGAGCTCGGGTCC
GCTAGGCCGCGGGTGGCCAGGGATTCACGGGGCTGCCCCGTTCGGCCGCGGGGAAGGTCG
GGGGCTGTGCGCCCCGCAGAGCGCCCTAGAGGCCGAGGCTGGACTCTGTGCCCGCGGGAC
CGCTGGATCCCTCCCGCAGATCCTTGGCCTCTGCTGGGACCAGGACGCCTAAAGGGGTTC
CCCGGGGCACAGTCACCAGATGTCTGGGCGCGTGGTTTGCGCAAAAGTTTAAAAGCCCAG
AGAGGAAGAGAGGGCACTGCCCAGTGTTGGAACATGCGACTCCGCCTCCAACCAGAAATC
CCTTTTAGACCTTAAGACTATATTCCCCTGTCTCCCGGGTGAACTTCCAAAGTCCTCGGG
CAGCGTTTTGTGCGTGGAGCTTCGCCGCCGTGGTAGTAACAGGTGCGGGGGTGGGGGATG
GGGAAGGCTCACACCGCCAGAGTAGTCCGCGGCTCAGAAAGTGTACTCAGGAGTCCTGGC
TTAAGGACCGAGGGGTTTGGAGAGTTGGGCCCCCAGCTGATGTTTCTGCATTGGATTGAA
AGTTAGGGAGCGAAAGGTCTGTAGGGCCCAGGTCTCTACCACAACCCCAGGGCAGAAGGG
AAGCCAGGTCCATACCTTGATTCAACTCCAGGAAACACCGAGCCGCGAGTCTGTAGGGCC
GGGACATAGAGAGCGAAGGTGAGGTGTACCTGAGGGATTGCCTCATAGGTGGAGCGGTTG
CTGTTTCTCAACCAGCTGCATTGGGGGTTCCAGTGTGGGTGACATCTTGTGGTGAAATAC
TGTCCCCAGCATCTATGTTGTGCTGTTGTGATTGTAGTCAGGGAGAAGAAAATGAAACTT
GGTTTCAAGCAGTGGTTCTCAGCTTAGGGGTGCTTTTGCCCGCTAAGGATATTTGACAAT
CCCTGGAGACACTTGGCCATCACAGGTCTGCCCCACAGTACAGAATCTGGCCCCTCAAAA
GTCAACATTGAGTGACCGTCCTATGACCACCAGCCCACTAGGGTACTTCTTGTGAATTTC
TCTCCCTACCTAAGTTTCTCCAGGGGCTGGGAAGGGCCAGGACGAATCTCAGTGAGAGAT
AAAGAGATAGGTGGGCAGGCTTCGCCCTGGCTGGCCACTGGCCCTGTGGAGGAGAAGCTG
GGAACAGTGGCTCTCTCGAAGCACAGGTCTGTAGTACTTTACCCAGGATAGCTTCAGACA
CAGGATAAGCTCAAGGTAAGCCAGAGTAGCCCTGGGGATGGAGGAAGGGCAGGACCAAGC
TCTGTTCCCATGGAGTTTCCCAAAGCTGGATGAGCTGAGGTCTCCCGGATAGCGGAATGC
CATGTGACCAACTGGAATTTTCCTTCTGAACACAGAACTGACTCCCCTAGCTATTTACAC
CAGGAAAGTAACATCCAAGGAATAACGGGTCCACTGATTCCCTGTGGCTCCTCTCTTTCC
TTCCAACAGGATGGGTTGCCCCTGGGGCGGTAGGAATCCTGGTCAGGGTGAGCTCAGGCC
CTGCTGTAGTATTTGCTGAGTGACAGTAAAGGATGGAGGCTGGTAAAGAGCTTTCCACCC
ACGGGGTCCCCCAAAACCGCGGAGTTGGGCTCCTGGGCTGTACTCTTAGCTTTCTGGGAA
TGGAGGTGAGGCTGTTGCTGGCTGGGTAGGTCAGGGCCAGAATCCTCTCTTCCGGCAACT
AACATTTCCATCTCCCTTTGTCCTGTCTAGTTTTGGACACTTCTTGCTTGAGAGCCCTGG
TGGGGTGAGAAGGGAGTGGTGGGACTGGGGGCGGGGCAGGAGTCTCGGGTTGGTTGGTTG
GTTGATTGATTTTGGTCTTTTTAACCATAAAACCCTGATGTGTAGCTAGACTTGGTGGTG
TGTGCTTTGATGTCCCAGCATTGGCAGGTAGTGGCAGGAGAGTCAAGAGTTTGTCACCCT
CAGTAACAGAGTGAGTTTGAGGCCAGTCTGGGCTCTACAAGACCCTGTCTCAGAAACCCA
GCAGAAGACCAGACCTTTACAATGTAGCAGTTACCACTACTGTATATGGCTCTGAAGTCA
TAAAGTTAAACACCCAGACCTCCTCAAAGCCACCTACCCCAAACCTGTAACTCTAGCTTC
ACTCGCTCATTCTGGTCAGTGCCCACCCCCACCCCCACCCTTTCTATACACGCTGCTGCC
AGGGGAGGGGAGGAGACTTCCAAAAGAGCAGGGGTAAATCACCCCAGACTGGAGCAGGAC
GACCACTGGGGGCTCAGGCCTACTCTGGGCTCACTGATGTTTTTCTTGTGACCTGAGCTC
TGGACAGTGCCCTCCTTGGTTGTGTGTCTGTTCAGCTTGTGTGTGCGGGATGCTTGCTAA
CCCCCCCCCCCCCAATCTGGAATTACAGACAGTTGCCAGCTGCCCTGGAAATGCTAGGAA
CCAGTCTGGGCCCTTTAGAAGAGCACCCGGTGCTATTAACTGCTGAGCCATCTCTCTAGC
TCCATGACTTGTATAAACTGTGTCCCAGACAGGCACCAGATGACAGCAGGAAGATGTCAA
GGGGCCGGCAAGTGTTCATGTTTGTGTTTGGGTAACTTGATGCTGGCTTCTGGGCCTGGA
TCCCCTTACACAGCATGTGGGAGGTGTGTTCTCCCTGCCCCAATCCAGCATGTTCCTTGG
AACTCATGGGATCCTGCCTAGATATTGCCCCATTGCTCAAGGGGATTTCCAAAGTGACCT
CACCATCTGTGCCGTTGGGAGCAGCTCCTAGTTTCCTATCCAGCTCAGACAGGCTGGGGG
AGGAGTGCTGGCTGACCTCAGCAGCCAGCATGGCCCTGGGACAGGGACGCTGCCAGGCCG
GGCAGCTCTTGGCACACAGGCAACCTCTCAAGAGGAGCCAGCCACGCCTGCCCCGTTGTT
GGGAGGAAGGGACTGCCCCAAAGTGTCCCTGGCCTTCCCAGGCAGCAGACCAGCATAAGG
GAAATCTCTTCTCATCTTCAGAGAGCTATGGAGAGTCACTGGGCACCATGCTCCCTTCAC
CAGATTTATTCAGGGACCCAGATGTAAGCATTTGGGTTTCAGCAGCTGCTGAAAGGGACT
GTCAGCTTCACTGTCCTGGCTCCCCGCTTAGTGGTTTGAGCCAAGTGAGTTCTGGCAGGG
TGTGGGATGATAGACACCATGGTTGGCTAGAGGGGCAGGTGATTCCGCGCTCAAGGCCCA
GGAGGGACGGTCCTGGGGCCAGCAGACTTGAGTCTGCAAGAAGGGTGGGGGTCACTTGAG
TAGGCTTGCCTCAGTTTCTACATAGCTGTGTAGTGGAGTCATTTAAGTAGACTAACCTTG
GTTTCTCCACTATACCGCGGAGGATTGAGGTGGCCTGTGGACATGTGGGTGTGACTGGTG
GAGATACTGAAATGAGCCATCATTGCAGCAAGACCGCTGTGCCTGGCACAGGCTTGGGTC
ATGGGAGAAACCCTGCCTCGTAGGCTGCTGGGATATCAGAACAGAAAGCATGTCAGGGTG
ATCTGAACTCTACAAGGAGCAGTTTCTGCGGTAGAGAACCCCCTGGGGCTGGGAGTGAGC
GTTTTATTGCAAAGCCCTGCTCCCCTGCCTCAGTAGGTGGCCACTGAACCCATGTCCACA
GTGTCACGGTGATGTAGGGGAGCTACCCGCCCCTGGTCCCTGGCACAGGGTGCCTTTTCC
CTCTCCAGGTGGGTCCCTGAAGGGTGCAGAACCTGTTGTGGTATTCTGGGTAGACACACG
ACTGAAAACTGAGGGCTCAGCTGGGCCCTTAGGGACTTTATCTGTCAGGCTGCATAATGA
CAGTGGCCTCCGCCCTCCCTCAGTTAATGAGATAATGCTTGTGAAAGCTCTCTGATAGCT
GAGTGGCTACACCGGCAGTAGGTGTTGCCACTCCTCGATCCTCGTGGATGCTAGAGGAAA
GCTTTCTGGAGCCGAAGGCATTTGCACCCCAACTACACAGATGGAGCAGCCACTCTAGAC
ACGCCCTTTTGCCGCTTGGCTTTTCATGTTTCAAGAGGGGTAGGTTTAAGAACTAGTGGA
GAGCCGGCTCAGGGGCTGAGAGCACACTAGCTGCTCTTGCAGAGGACTTGGATTCAATTC
CCAGCAACCACATGCTGGTTTGCAAACATCTGACATTCTGTTTCCAGGGTACCCAACACT
CTCCCCTGGCCTCCACGGACACTGTACGTGGGGTATAGAAATACACACAGATAAAAATAC
CTGTACACACAGAGAAAAAAAAAATAAAAGTAATTCTTTAAAATATAAAAAAAACCTCAG
GACCCAAGCAGGCTACAGCCCCAGCGCCTCCGGAGGTAGCGCAGAGACTGAACATCAGTG
AGCCTTGATTTATAACTCAAACTGTCAGGGAGAATCTTGTCACGTGGCTCTTGGCTCGTA
GGCTGAGTGGGTGGGTCATGGTGCCAAGGGGGAAAAGCCATTAGTTAGCTACTGCTGGTG
CACCCGGCATTGTACATGCAGGATCTGCCTGCCATCCATGTAGTGACTGTCACCCCATCT
CACAGAGGAAACCGTGACAGACCACACGTGAGCCTTGACTCTGACTCCCAGCATGCCATG
AGAGTCCTACACTGCCCCAAAGTAGCAGATAGCATTGAAGGTTCACCTGCTGTGGGCACA
GCTTGGATGGCCCTGTGTCCAAGTTCCCTTGGCACCCACTGTGACCGGGACCAGCCGCTT
CCTGGGGAAGTAGGGTGCCCCAGGGCAGAGTTCTGGAAAATCAAGTTTATTAGCTTCTTA
ATGACGGTTTACAAGCCAAAAAGTGTTTAGCTGGCGTGTTCCCTTCCTGCTGAGAGGCAT
CAACCCTGCAGAAGGAGATCTGGGCAGGATGCCGAGCGCCCTGTCCTGAGTATGCTGAGG
AAAACATCTCAGGGCAACCTGCAATCCTTTGGTGCTTTAGAGCGCTCATACCTGACCTGG
ACACTGCCTGTTTCTGGCTGAAGGACCCACACTATGCAAGGACTCTGTAGGATAGGGTGA
GACTTTGTCCCTCAGTGGAACATGCTGTATGATAGATGAGCCCTAGTTATTGGTGACAGA
AACACCCAGGTCTCAAGAGCCCACATAAACAACACATTGAGTTAGAGGTATTAAGTAGCT
TGCTCACAATCACACAGTAAAGCTGGCCCCGTGACCCTGCTTCTCCTAGTCAGGCAGTTC
CTATCTCTGTAGCCACGCTCCAAGATGGCGGATGGAAATTGGGTGGGCGATTGGCTTGCC
AGTATCAGGCGAAACCCCAGCTGTGTGGTGTGTCACATCTTGGTGTCAGGGAAGCCTAGA
GAAATAAAATCACCACGTTACTCAGCCTTCCCTGCACCCACCCCCTCCCAGGCTACACCG
CATCCTGCCCTGACCACTGGGACTGTACTCTTCTGGTTTTGAAGACTGATGAAAACCCAA
TTCCCAGTAGCCAATGTTCCTATGACAGGCAGAGCTTCCATATGAAAGCAAGCTCATGTT
GATGTGGTGCCCAGGGTAAACCGCTCCCTCTGTGGGAGCCTCCTTTGACCTGGCGTCTTG
ACCCCCAGGGAGGGGCAGCAGAAGTGGGACAGAATATATAGTCTTCTCTGGAGACCAGAC
GGCTAGACAAGCAGCCTAGAACCCAGCCGCCTATTGGCTGTCTCTGTACTGTTCCCTAAA
GCTGTGGTCAGCATCCCAGCCTGCAGCCCTGCCACATGACCTGCTGTTGCTGCCTGAGGT
GTGAGAGGGCTCAGTTCTACTCAGAGCACCTGCATCTCTGTGACTGGAGGGGACATGCCT
CGCCAGCAGCTTCTAGAGTCATGAAGTTTCCAGAGGAGCTTTGAGCTGCAGCTCCTAGCT
GCATACCCATTTCCCAGTATGTTAGCATCTTGTGTGTGTGTGTGCGTGTGCGTGTGCGTG
TGTGTGTGTATGGAATCCAAAGCCTTACGCATGCCGAGCCAGCTTTCTGCTACTGAGCTA
CACCCATGCCCAATTGTTGTATCTTGAGCTCTGGGGATACAGAACCTGTCCCCTGCCTCA
ACTGCTCCTAGGACAAGAGTCCCCAGCAGAGGCGGAAGGCATTTGCACCTCTGCTGATCT
GGCTGGCCAGTAGTCCAGAGAAAGGCCAGATAGATGGGGAGAGGGGGAACCCGGGCTGGA
CCTCTCTAAGAGCAGGGTGGCACACCCACACAGACAAGCTCACATATGCTCTGCTCTTGG
CAGCTCCCTGGAACCTCGTGCCCACTTCCCGTGCCTGTGGTGGGCCGTGATGCCCAAGCT
GCTCTAGTTGGGTCAGAGGGAACCGAGAGCGTAGGGTCTGAATGTGGAATACCCCCAAAG
AAGCCAGCAGGCTGCCAAGTCCTTGAGCCTGCTCCTGTAGGTCTGGTCTTGTGAAATAAG
TTTGGCACAAGCCCTGGGAAGAAGGGGAGAATGAAGTGAGGGCTCTGTGGGGGAGGCCTG
TTGCTTGTGAGGTCTGTGCAGCGTGTGAACCCACAAGCCAGTTTTCTTTTCTTGTGGGTG
GCCACCAGGCCTGTGCAGGCCAGCAGAGCGTGGTGCCTTGGCATACATAAGGCTTTGGAT
TCCATACAGCCATACACACACACACACACACACACACACACACACACACACACACGCGCG
CCACATTTGTGCTACCACCTTTGGCCCATACCTCTGAGGAAAAGCACTTCAGGAGCACAG
AGCCCTCTGTCCTTTCTCCGTGGTCCTTGCAGCTTCCATTCGATGCATCCTGCTGCTAGA
CCGCAACCCCCCCTTCTGTATTTTGTGACCTTTTCTGGGTCCTGGCCCCAGACCTGGCCT
CTTCACCTCTCTGCCTTTGGCTCCATCATTCCTGCCTCTTTCCAGGGCCAGGAGATGCTC
TGCAGTGAGTCACCAATTTGGAGACTTCTTAGATGACAGTCTGTCTTACATGGGTTAGCC
TTCCACGGCCACCAGGGCTGTCAAGCTTTGGGAGGCCAGCAACAGAAACTAACTTTCTTT
CTTTTTTTCTTTCTTTCTTTCTTTCTTTCTTTCTTTCTTTCTTTCTTTCTTTCTTTCTTT
CTTTCTTAGCGGGATTTCAACAGTTGAGGGTTCCACTTCCTCTCTTCAGCTTTCGAAAAA
CTCAGAGGTTCCCAGCAGGGCCACAGGAGGGAAGATGCAGTTTTGGTTTCTGTGGCAGGG
AAACCGGGGAGAGAAGTTGCTTGCTTGCCCCCAGCCCACACTGTCATCATCCTGTGACTC
AGCAAGGACATGGCATATGGTCTGTCTGCCACCACCCCCTTCTCTGCCCGCTCTCTGCAT
GTCCCAGGACGTGGCTGAGTGAGGCTTCTGGGGCAGGTGTAGTTTATTAAGCCCTCTGTG
ACTCGTAACACCCCCCTTTGGCCATATCCAGTTGTGAATAATGGTCTTCCCCCTTTCCAT
GGAGGGGAGAGGGCTACCCACTTCTTGTCCATTGGTCCCTCCTGCTGCCCACATTTCTGG
CCCATATGTGACCTTGGCTATTACATCAGGCGCCTGCTTGTTTGGGAATACAGGCTCCCT
CAATGTCCTGTTTCCTCAAGTCTACAGAGCTTACGGGTGTAGGGAATCATTAATAACGCT
GGGCTTCCAGGATAGTGGGGGTGCGTCAGGTCAGACCTGTGGGTACAGGTCTACCCCTTC
TTGGAGCAGAGAGGGAGGAATCTAAGCATATGCCACCTGCATGGGAAGGGAGGGCACCCT
GCCTAGAGTGGTTGGGGGAGCACCCCCTGGGGGTGACCTTGCCTACCATGTTATCATGCC
TGGATTGGACCTGCCTCATTTGGGAGTGGCTGGGCCCCACCCTTTGTGGGGAAACCGGAT
TCTGTGGGACATTCTAGGTAGTCTCAGAGCCTTTGTTTCTGTGTGCCTAATCTGGGCCAC
CACTCTGTGGGTGGTGCTATCCGGAAGGCTCTCTGGAGAGCACGCTGACCCCTAGTGGAG
GCCATCTACCAAGGGCTCCTGGGGCGGGGGGTGGGGGTGAGGGAGGCGGGGGGTGGGGGG
GAAATCTCAGCTTTGCACTGCATTCTTCTGCTGTCTGCCCCACCCCCACCTCTTGGTAGA
CACCACCCTAGAGGAAGAGGAAACACTGACACCCCTCCGCCCTGGGTTCCGGGTAAATAT
TCTTGTGAGAAAGACTTCTCCCTAGAAGTTAGACCAGGATCCAACTTGAGCAGGTGGCAG
AGACCATAATCCTGGCTTTGGTCCCTGTCACTCCCACCCATGGGCCAGCTGGGCAGCAAG
TCTAGGGTCAGAGAGCTCGGCGGATATGACTGTGGGCAAGAGGAAGCTTGCTCCCAGGAT
GTCACAATTTCCTTTGCAATGAGCAAGCACCTCTGGGGGCGGGAGATAAGTGGCGGGCGC
GGAGGGCGGCTCCTGCAATTTTTTTTTTTTTTTGGTCTAGTTTCTGTGGCCTTTCATAGA
CGTAGGTCACACGGGGGGACTAGAGCAAATCTGATCATTCCCACCACTGTGCCAGGCAGC
TGCCAGCCCGCCCAGTCCAGAGAAGGGTTGGACTTTACCCCCCCCCCCCAAGGTTGGGGC
AGAGGGGTCAGGAGAAGTTCTGGGAGAAAGTATCAGAGAAAAACCTTCATTATCCAGGCT
GTCTGTGGTCAGCAGGTCTGAGCCATCACCTTGTCCAACTGCCAGTGGACAGGAAGGGAA
ACTGAGGCACAGGGATGTAGCTTAGGGACCATCATTCAAAGCTCGGTACTGGCTTCGCAG
ATGGTGGTAGAGAGTTTCCTGGCACGTGACTCCTTACAGTCTCTGACCCACCCCTGCGGT
CGGACTTCTTTCTAGGGGACCCTGAGATTGTCCCAAACTTCCCCTGTCCTGTGGCCTTAA
GTCATGTCTGCCTTTTCCATGTGGAGATTGGTTAGGGGGGTGGGGTGGGAGGGATCCTGG
GTATATTTGGGGGAGGGGTTGTCACCAGCTGGAATGGCCTGGGCTCTAGGGGCAGCTCCT
CAGAGGGGTAAGAGTGTTTGTGGACAACCAGGGAACTAGGCCACCAGGCCTCATCCTCAG
CCAGCACCCAGAGACCTCCATCCCAGCCCTGCAGAGGGACTGGGCAAGGTGAGTGACTTC
CAGAGCCAAGTTCAGAGTTTGAGAGACAGCCTCAGCCCAGCATCCATGCAGTCCGTCCCC
AGCTGCACAGACTCCTGCCCTCACCAGACTCATCCAGCTAGGACTTGATGGGCAGTGATT
GGGTGGGAGATTAGAGTAAGGGGCTCCTGCTGTCAGGAGCTGTGGGATGCAGCGTGAGAC
AGAGCAGGGTGGTGAGAGGCTTCCTGGAGGTGACACGCCTTAGAAGCGCAGTAAATACAA
AGGTTGCTCCCAGGGCACTGGCAAGGCAGCTGATGCGGAAGGCAGGGTGGCATGTCACAG
CCCGGAGCCTGCACAGGTCCTACCCCTGACACACTGCCAGCCGCATGATCACCTAGACTC
TGAGCAACAAGAAGACACCAGAGATGAACAATGGTTCATTTTCTGGGTTAAGGAAAGACC
TCTGAGGTCCATGGTCTGTGCTGCCGCCAGAGACCATTGTTACAGTCTGCCGTCCATGIT
ATCTATGACCATTGCTGTCACCAGAGGCCATGTGGATGCATGTGAATGTCCCTGATCCCT
GGCCAAGCTTCTGTGGGCAGAAAAGCTTCCTCTGCAGTGATATTAATGTCTGTAGAGTCA
TAACTGAGAGTGAGCGACATTGAGGGGCTTCTGTGACAACCGCCCCCCACCCCTGAGAGA
GAGAGAGAGAGGGAGAGAGAGAGAGAGAGAAAGGAGCTATTGAAGAGAGTCTGTAAAAAT
TGTCCAGGCAGTGGAGCCTTTAATCCCAGCACTGGGGAGGCTGAGCCAGGTGGATCTCTG
GTGTGTGTGTGTGTGTGTGTGTGTGTGTGTGTGTGTGTGCGCGCACGTGCGCGCGTGTGC
CTTIGTGTTTTCCAGGACAGCCTATCTCAAACAAACAAACAAAAAGTGATAAAGATGCTA
AGTGTGGCTCTTCACAGTTGGTGGCTTCTGGCAGGAATGTGGATGGGGGAAGACTCAGTT
CTCTTAAGGGGCTGGCCACTGAGAGTCTGACGATGCTCCAGTGAGTACATGGGTAACATA
AATTGAACTTGGTAGATGTGGAAGGAACACAAGGGGAGAAAGGTGTICTGGAGGCGTGGG
AAGCGAGAGGGGTAGGAATACATTTTATAAAATTCCCAAATAATTAATAAAAATATTTTG
TTGGGGAAAAAGAAGCAGCAGAGGTTGGCCAGGTTGACAAGGCTGGCATAAGATTGCAGG
GCCTCAGAGACAGGAGGTGGGAGCTTGGCAGAGGTGCAGGGGAGAGATCTGGGTCTTAGT
AACTGTTCTCTTGCTGTGAAGAGACACCATGACCACGCAGCTTATAGAAGAAAGCATGAG
TTGAGGAGTTGCTTACGGTATCAGAGGGTGAGGCCATGACCATCATGGCAGGGAGCTTGG
TAGCAGCAGGCATGGTTGCTGGAGAAGTAGGTGAATCTTTACATTCTGGTCTGTAGGCAG
CAGACAGACAGAGAAAGAAACAAACCTGGCCTGGTGCAGGCTCTTGAAACCTCAAAGCTC
ACTGCCACAACACACCTCCTTCGACAAGGCCACACCTCTCAATCCTTCCCAACCAGTTCA
CTGCTTCACACACATGAGCCTGTGGGGCCATTGGCATTCAAACCATGACAGTCTGGCTGG
CTGAGTGGGGCAAGCCGGCCCTGCTGACTCCTGGGCAGGGCCTTCAACTCCTCATTCCAG
AAGGCTCTCATCGCTGTCAGCTAGAGAGCCACGGGCGGTGTTCTCAGGGTGCAGAAAGTT
GGACCCATCTCTGCTGGTGACCTTCTTTGCCCCCCCCCCCTCTCTCTCTCTCTCTCTCTC
TGCCTGCCGTTCTCCCTTGGTGCCAGTTTCTCAGAGTAATGCTCTCTCCCTGCACCCTGG
TCCCAGGCCAGTTGAGGAGCTTCTATGTGGAGGCCCCAGGAGAGGCTGTTGTCCTGGAGC
TCTCCCCTGGCAGTGTCTACGTGTGACTAGCAGGGCTCCTCAGAGTCTGCCAGTCCAGCC
CCTAGTGTCTTCCATCTGTGATCTCAGAGGGCTGAGGGACAGGACTCTTGGCATGATGGA
GCCAAAGCTAAGGCCTTTGGTCCCACTTAAAGAAGCAGTAATGATCTGGGCCCGCTCTGC
CTACTCCCCACCTAATGCTCTGGGTCTGTAAAATGGAGAGTTTTACAGGCCTCAGTACAG
CAAAGCCTAGAAGGGTCTGCAGATGGGATCACCTGGGCCTACAGCAGCCCCTGCTTCCAG
TTCTCCCATGGTAGGCCGCCTCTGGGCAGCCTAGGCCTCTGCCAGTCATGCTTTGAGAGT
CACTATAATGCTAGACCATAGCTCCCTGTGTCTAAGATAAGAGTCTTCCAGCCCCAGTGT
ACCCTGACTTTAGGAAAGGAGGGGCCCAGACTCCTAAGGTGGCTCACACCCACTGTCAGA
TGACTTGGATCCTAGGGGCACCCCTTCCCTGCTGTGTGAGCTTGGTTAAGTTAATCAACC
CCTCTGAGCCTACAAATCAGATGCATAGCAACATCTCTTCTGGAAGGATTTGAGGGGCAG
TGGGTGTGCGTAGGGCTGAGCCCGGCCTGCCACTCATGGCACTAATTGTGCCCGGATACT
GTACCACTTCCCAGTTGCTCCTGGAGCTCACTCCAAGGAGAACCTGCTGTGCTCCTGTGC
CTTGTTTGTAGTATACGCCTGCACAGGGCTTTCTACAACACAGGCACCGTGGTGTTCTCT
TTCAAATGGTTAATGTTCTGTTGTGTGGATTCTACATCAGTAAATTATTAAGAAAAAGCA
AAAACAAACAAATGGCAGGAGGGGTCTCCATTGCCCTAGTACCAGGCCACCTCATGGAAA
TTAAAGTTTGTGTTTGTAGCGCTGGAGGTTGAAGCCAAGGCCTCCTGAATACGTGGAAGG
CACCTGACATCGAGCTATAGCCTTGGCCCCTTTCCTTTCCTCTTTCACTTGTTCACACTC
GCATCAGTGGGGTGTGTCTGTCGTGGCGCGGGTAGAGGCCAGAAAACAGCCTCGCTTGCT
GTGTGTACACCAGGCTTCCTGCCCCTAGGGCTTTTGAAGGTCCTCTGTCCCATTGGAGGC
ATGTGGGGTTCCAGGTGCTGCTGTTGGTGGGTCTGCATGGGTTCTTCACACCTCCATGGC
AAGCACTGTATCTACTAACTCACCGCTAGCTCTAGACCCAGCCCTTTTTCCATCTGTGTG
TGTGTGTGTGTGTGTGTGCGTGTGTGTGTGTGTGTGTGTGTGTGTGTGTGTGTGTGTGTA
CAACCTTGGGTGTCGTTCTCAGGGATCATACACACACCTTTCTTGTTTTCAGATAGGGTC
CCTCAATGCCTTGGAACTTGACCTCTGGGCAACCAGCCCCATGTATCAATTCCTGCCTCC
ATTGATCCGGGGCTGGGATTACAAATCCGTAATGCCATGCTCAGCTCTGTTGTTGTTGTT
GCTGCTGCTGCTGCGAATAAGGATACTGGAAATCAAACTCAGGCCTGCGTGTTCTATGGC
AAGCATGTTAGGACTGACCCATCTCTCGATCCTCAGAAATCCCAGCACTCGGGAGGCAGA
GGCAGGCAGATTTCTGAGTTGGAGGCCAGCCTGGTCTACAAAGTGAGTTTCAGGACAGTC
AAGGCTATACAGAGAAACCCTGTCTCGAAAAAAACCAAAAAAAAAAAAAAAAAAAAAGAA
GAAGAAGAAGAAGTAGTCAGGGTAAAGTAGGTGGGTCGGCGGGTAAAAGTGCTTGTGGTT
GGGGCTGGAGACATGGCTGAGTGGTCCAGTTCCCAGCACCTACACGGCCACTCACAAACA
TTGTAACTCCAATTCCAGGGCATCCAACGCCCTTTTCAGGCCTCTGTGGGCATCAGGAAT
GCATGTGAGGCACGGGCATACATTCAGGCAAAGCACCTACACACGTTCTTCCCAAAAGTG
CTTGCCACGGAGGCTTGATGAGCTGCTGCCCACAAGGGAGGAGAGATTAATTAATTTTTT
AAAAAAAGCCAACAACGCTGGGCGTGGTGGCGCACGTCTTTAATCTCAGCACTTGGGAGG
CAGAGGCAGGTGAATTTCTGAGTTCGAGGCCAGCCTGGTCTACAAAGTGAGTTCCAGGAC
AGCCAGAGCTACACAGAGAAACCCTGTCTCGAAAAAAAAAACCAAAAAAACAAAAAAAAA
AAAAAAAAAAAAAAAAAAAAGCCAACAACAATTCCTGGATTTGTGAATTGAATATTTCTA
GTAAAGCGATAGTGAACTGGGAGTAGTGGCTCACGCCCGTCATCCTAGTGCTTATGAGGT
AGAGCAGAAAGATCAGAAGTACAAGACCATCCTCAGCTACATAGCAGGTTCAAGGCCAGT
CTAGATGCATGAGACCGTATTTCTAAAGGCTAAAATGCTTTTTAAAGGTAATATTAGAAA
CAGAATAGGATAGGGCAGAGTTTCATGCCTGGTTCCATGTCTGAGTGGTGGCAGGTCAAC
ACTCCAGGGTCACTGCCACCTGGATCAACACTACGAGGCGCACACACAGGGTCTCCTGCC
CCTATGGGAGATGGGCGGATACATGGAGAGACTAAGGTCCAAAAAATGGGAAGATTTGGC
AGTTGGCCTGACCCCCTCACCTGCGTGTCTGTTCTCACAGGTTGGCCTGCTTGGGCGGCT
GCCCTGCCTGACCCACAGCCATCTGTCAGTCCATCTGTCCGCCTTGACCCCAGGAGACC
C
AGAGATCGAGGAAAGGATCGGAGCATGGTGTAGACACCCTCAGCCAGCCCAGGGAGCC
CG
GCCCCGCACATCTGAGGGAAGGAAGTGGCTGGCCAGGTGAGTGGTCTCGAGACCTCTACT
CCTCTATTCGGTTTGGGGACAATGGCAGGCAGTGGGGACAGGTGCATCTCAGGGAGCAGG
GAGAGCCTCTCTGGGGAAGTGGCATCCAGGTAGAAGCCAGAAGGTGCTCTCTGCCACATG
GTGGTCAGCAGTCAGAGTGACCGGTGCAGGCAGAGACCCAGCATCTGTGGGCGCCTGATG
GAGATAGAACCCAGGACTGTGAACACGCCAGACCATGTGCTGACCTCTGACCTCTGTCTC
AGCTTCTATTTGTTACCCTTTTTATGTGAGATAGGACTTTGCTTTTATAGCTCTGGCAAT
CTGCCTGCCTCTACTTCCTGACTGCTGGGCTTAAAGCCTTGTACCCACACCTCTGGTTTT
TGTTTTTTTTGTTTTAATGTGTGCATTCTTTGTATGTGTGGGTCACAACCTGGGTCAGCA
CCGTGATCCCTTTGTATTGAGTGGATATCAGGATGAGATACCATATGGATCCGCACTGTC
TGTCTGTCTGTCTGTTCAATCCTCTGGTAGCTGTCACCTTCTATCTCTGGTGAGCACGCT
ACTGGGATGTTCCAGTAGTGTGTGAGGCCGCGCTGGAGGGATGCTTTGTGGGTTCTTTGC
GCATAGAGTATCTTGTCTTTGTTTCATGGGAGGAAGCCTGAGTGACGGTGATTCTGGGCT
GGCACTGTCTCTCTGTGTGTCTGTCTGTCTGTCTGTCTCTCTCTCTCTGTCTCTCTCTCT
CTCGCTGAGGCAAGCCAAAACTATCACAGGTCAAGCTGGGGGCACCCACAGTCTGCTGAG
GGGGTGGATAGTCGGGACAAAGCTGGGGGAGGGGAGCTCATGTGGCTGGAGGCCACATGG
AGGAGCTCAGCCTTAAAGAATAGGAAGTCAGGCTGGAGAGATGGCTCAGCGGTTAAGAGC
ACTGACTGTTCTTCCAGAGGTCCTGAGTTCAATTCCCAGCAACTACATGGTGGCTCACAA
CCATCTGTAATGGGATCTGATGCTGTCTTCTGCTGTGTCTGAAGACAGTGACAGAAGGGT
GAAGGTGGAGAGAGCTAGGGGCCGGAGCAATGGCTCAGCCGTTAAGAGCTCTTGTTCCTA
CAGAGGACCTTAGTGTCAGTCCAGCATGCCCGTGGTGGCAAGCAGCTTTCTGTGACTTCA
GTTCAGGGATCTGTTGCCCTCTTCTACCCTCCATAGGCACCAGGCATACCTACTGTGCAC
TAATAATGTTCATGTGCAGTCACCAGGCATGCACACAGAGTACATACACACATGCAGACG
AGCTGGACATAGCCTCCGTGTGGTGGCCCAGGCACCACCCACTGGCACTTTGCTGGTCAT
TGACTCGTGAGCTTCCCAGAGCTGCAGGGAGAGAAACAGACAGCACATGCCTGGGAGACA
TCAGAAGGGAGTGGGGCTCAGTGGGGTTCAGTGGCTCGAGCAGGGAAAGGTGGCCTTCTC
TTTACAGGACTGTCTGTCCAGATCCACCGAAATGGCCCACCGCCTTGCTCTTCCCAGCCT
CCCACTTCTAGATCTTTGTCTTAACACGCCTCACAGCATGATCAACAGTAGACAAGACAG
CCTGTGTGCCCATCCATCAGGAATGGAGTCTTGGTGTGTTCCATTTAGTGAGGCCTTTCC
TGTCAAAGGCAAAGACGCCAGTCTGTGTGTCCTCAAGGGAAAGACGCCCAGACCAGACCA
GAGAACCTCAGCTGTTTTAAAAAACCTGTGCTGTTTCCAGGAGAGAGCGGCCGCCCTGAG
GGATGCTCAATGTCTGCTTTGCCACACTCAGTCTTTTTGTTTAGTGACTGGGATATGGCT
GATTTGGGGGAGCGCTTACCTTGCAGGCATGAAGCCCTATGCTTGATTTCCAGCACCACA
TTCACAGCTGTAATCCCAGAGGCAGGAGAATGAGGAATTCAAGGTCCCCCTTAGATACAG
CAGGTTCAAGGCCAGCCTGAGACGGAAGGAGAAAATGAGAAATCCCAGGCTAGTGGTAGT
TTCTGTTATCTTTCTCGTTCCTTCGCTTCTTTCTAACAGATGTAGCCCAGGCTGGTCCAG
ACTGTCCTCAAACTCACAATCTTCTGTCCTTGGTCTTCCAAGTACTGGGATCAGAGGCTT
GTACACAGAGGCCTGTTATGTTGCTAGGCTAGCCTTGAACCCCACCCCACCTCCGGAACC
AAACAGCCCTCCCTCCCTCAGCCTCCTGAGCAGCTGGGATCACAGGTGCACACCACTAGA
AGTGGCTTCCGCTTAACTCCAAGGAGGGTCTAGGCACACTTATGGGACAGAGGACCTGCA
TGGTGGGTTTCTCTTCCCTCTGGGAATTAGACTGAGCTACCACTTCCTGTGCATAACTCT
AGGCCTGCTTCCCCAGGAAATGCACCTGCCCTGCAGGGTTGATCGCTGGTCACAGCAGCT
GATGCCTGCCCAGCAACACCAAGAGCACTTTATTGGCAGTAGTGTGTCTGGTTGTCCCTT
GAGCCTCTCCCCCAGCACCAAGGAAGGGCCCTTTGTCTGTGCCCAACACCCTGGCCACTG
ACTTGCTCCGACCACACCCACTTCTTTCTGCTCCTTTGGTGGTTCGGTTCTCACCGAAGC
AGAGAGACCGACCACCAAGGGACTAAGGCAAAAGTCAAGGTCTTCCTCACCCCGGCGAGT
CTGAGATGTAAATCAAAGTCAGAAATAGACCCCAAGATCCTCCCCCCTCACCCCACCCCC
CCCACCCCCCCAACCCCCCAGCCCCCCCACCCCCACCCAGGTGTGGTCTAGCCACATCTC
TCTGGAGCTGGGCCTGAGACCACACGGGGCAGGCTGGTGCCGGTGCCGGTGCCAAGTGGC
CTTCCCTAGTGCCAAGGTCTCCATCCCCTCAGGTCCTGCGGCCCCTCTGGATGCCCGTGC
TGGCCTCTCCTCTAGCATCGTGCCCACCATGGCCTCAGCTGACAAGAATGGCAGCAACC
T
CCCATCTGTGTCTGGTAGCCGCCTGCAGAGCCGGAAGCCACCCAACCTCTCCATCACCA
T
CCCGCCACCAGAGAGCCAGGCCCCCGGCGAGCAGGATAGCATGCTTCCTGAGGTGAGG
GG
CTGCCCGCCACAGGCCACAGATGTGACTGCCCACACAGCAACTAGACACACTCTTCCATC
TCAGTCTACTGACAGTCCTGCAGCCTCAACATACGCCTGAAAGATTGCAGGGAGCAAGGC
TCTGAACTCTGAACTTGGGAGTTCTGTTGCCTGGTGACCAAGGGACAAGGAACACTGTCC
CTGAGAAAAGGCCTTGTTGGGAAGCCTGGGCTCTGGCTGTTTACAGTTCCCCTCCCTGGC
CCTGCTGTGGGCTGTCTGTGGAGGCCTGAGTGGGCATGGGGTCCCACAGTGCCAGCAGCC
ACCCCTACAGCGCCTTGTTCTTGGGGGGATGGGTCATTGTTTACGTCCATCTGGGACTCT
TGCCCCATAAAGAGCTCATTGAAAACAGGCCCCAAATAGCGTGGGCTTTGAACACAGCCT
GAAACATAAAGGGGAGCTATGTTGGGGCCACCCGCTCACCCTGAGAGTACTCTAAGACAC
GTGAGAAGAGGCAGGACATCCTGGTATATGGGCATATCATGGGTAGGGAGTGTTTGCACT
GAGGTTTGTACAGTGGAAAACTTGTGTTTTGTTTGTTGAGACAGGGTCTCGTGTAGCCCA
GGTTAGCCTCGAACTTGCTCTATAGTCAACAAGTACTTTGAACTGCTGGTCCTTCTTGCT
CTACTTCCTGGGTAGAAAGGTGCTGAGTGGCAAGTGTTCACCGCCGCACCGGGGGGCGGT
GTGTAGCGCTGGGACAGGAACCCAGGGCTTTAGGCGTGCTGGGCACACTGCCAACCTGAG
CTGTATCCCCAGACCTTGCTAGAAAATGCTCACTTTGGGTTCAGAATGAGGGCCTATGGA
GGACTGAGTGCAGCCAGCAGGCTAGCGTGTTAAAGGGTCTGGGCTATGTCCTCAACTCTG
CAATAAAAAGGGCTGAATGGGAGCCCAAAAGCAGCCAACAGGTGAGTGGCCTAGACACTC
ATTCATGTCCTCCTTGCCTCCCCACAGAGGCGCAAGAACCCAGCCTACCTGAAGAGTGTC
AGCCTACAGGAGCCCCGGGGACGATGGCAGGAGGGCGCAGAGAAGCGCCCCGGCTTCC
GC
CGCCAGGCCTCCCTGTCCCAGAGCATCCGCAAGTGAGCACCTGAGCCCTGCCTGGTCAC
C
CCAGCGGCCTGGCCTTTCCCGGGGCCTGAGCCCTGTGTCCCCCTTTCCAGAGGTGTAGA
C
AATCAGGGAGTAGCATCTCCCCGTGGATGTGGAAGGCCAGAATCTGAAGTGATGGTGTT
A
ATGGGATGAGAAGGGGCTGGGGCTGGGCCTGTGCACCTGACAGGAAGTTCCACAGCAC
GC
TGCAGAGGGGCTCTCCAGCCTTCCCAGTCCCACCCATCATGGGGTAGCCTTCACTCTCA
G
CCTGGTCGCTGGGCTGTAGCTGGGAACTCAGGGGGGTGTAGGGAAGACTCACTGATTC
CC
TAGGGCCTTGAAGATACATGGCAGGGTCCATGGTCCATTGGTTACCCTGTATACACACA
G
GAACACACATGCCTTACCCTCAAGTCCCCTCTCCCCACACACACCGCCATCCCCTAGAC
C
ACCTTCCATAAACACGAGCACCATGGCCTCCCGTCACCTCATACCCGTGTGCCTCGTGT
A
CCCGCAACTCACACATCACCCTCCCAACACACACACATGTACCGCCATCCCCCTGCCTC
C
TCCTTTAACACATGTACCTTCGCCCTCACCTCCTCGTGCACACGCGTGCACCAGGACAG
G
TGCGTGCACAGGTTTCTGCCCACACCGTATCTGTTCTGCAGGAGCACAGCCCAGTGGTT
T
GGGGTCAGCGGCGACTGGGAGGGCAAGCGACAAAACTGGCATCGTCGCAGCCTGCACC
AC
TGCAGCGTGCACTATGGCCGCCTCAAGGCCTCGTGCCAGAGAGAACTGGAGCTGCCCA
GC
CAGGAGGTGCCATCCTTCCAGGGCACTGAGTCTCCAAAACCGTGCAAGATGCCCAAGGT
G
GGCCCCCTGGAGGTGATGGGCAGCAAGCGGCTCTCCCAGGGTCTGGGCAACATTGTTCAC
CCACATCTCTTGCAGATTGTGGATCCACTGGCTCGGGGTAGGGCCTTCCGCCATCCAGAT
GAGGTGGACCGGCCTCACGCTGCCCACCCACCTCTGACTCCAGGGGTCCTGTCTCTCAC
A
TCCTTCACCAGTGTCCGCTCTGGCTACTCCCATCTGCCCCGCCGCAAGAGGATATCTGT
T
GCCCATATGAGCTTTCAGGCAGCCGCCGCCCTCCTCAAGGTAAGGTCTGCATTGAAGGAT
GTATGTCCCCGTCCAGGGTAGCCACTCCACTCACCTACATGTCTACCCTCTGTGTCAGAG
TAGTGAATGCATGCACACCCTAAAGGCCAGAACTTATGTCCTTCAGGCCCACAGTTCCCC
TGTGAGCCCTCAGCGCCTCCTCTGCTATGGGTTATGGGAGAAGTGGTGGGGGGGGGGGAG
GTATTGGATGTAGATGCCTTGAGTGCCGGGTGCCAACTGTTCCCGTTAGCAGGCAAGAGC
TCGTGAGGGAAGAGCTGAGAGCTGATCCTGATTACTGGAAGGAGAGGCTGTGTAGGGCCG
GCTGCAGCCAGAGCCCCATCCTGGCCTGCTCTGAGTACTTCCTTCAGCCCGTCTACTTAT
CCAATGTCCTGTGCCTTTGCCAACCTGGTCCTGGGGTTCTGAGCCCTCCTTGACGCTTCT
GTTCCATGCCCAGGGGCGTTCCGTGCTAGATGCGACTGGGCAGCGGTGCCGGCATGTCA
A
ACGCAGCTTCGCTTACCCCAGCTTCCTGGAGGAGGATGCTGTCGATGGAGCTGACACCT
T
CGACTCCTCCTTTTTTAGTAAGGCAAGCATGGGGTGTGGACTCTGGGCGGGGGATGGGGT
GGCCTGAGAGCTAGGAGGAGTGACCTTGGCCTTGACTGTGGCTTGTGGGATCCCAGGAAG
GTCCAGAGGCAGGGTGGGGTATGGCCTTTGTGAGCCATTGGTCTGGGCTCCCTTAGAAGG
GGGTGTGGAGTGGAGCACGAACCCGCTGGGAAAGTTAGGAGTGACAAGCATGCGGCTGCC
CTTCCATCCTGAAGAGACTGTATAGCTTTGCCTTGCCCTTGATTGGAGTGCTACGATGGT
CCTTGGGGCAGTGGCTCCTCACACTGTCCTTGGTGTCCCTCTACTCCAGGAAGAAATGAG
CTCCATGCCTGACGATGTCTTTGAGTCCCCCCCACTCTCTGCCAGCTACTTCCGAGGTG
T
CCCACACTCTGCCTCCCCGGTCTCCCCGGATGGAGTGCACATCCCGCTGTGAGTACAGG
G
TGGGACTCTCCAGCCCCTGCTGAGCCCTGGCAGGGTCCTCAAGCTAAGAGCCTCTGTCAT
CACCAGAAAAGAATACAGCGGTGGCCGAGCCCTGGGTCCCGGGACCCAGCGTGGCAAA
CG
CATTGCCTCCAAAGTAAAGCACTTTGCATTTGACCGGAAGAAGAGGCACTACGGCCTGG
G
CGTCGTGGGTAACTGGCTCAACCGAAGCTATCGACGCAGCATCAGCAGCACCGTGCAG
CG
GCAGCTGGAGAGCTTCGATAGCCACCGGTGAGCCCCCAGGAGCCAGTCTGAGCAGAGG
AC
TAAGTGGCTAGAGTTTCCAAACCTCTCAGGCCTCTTCATCCCAAACATGGCCCCTTCTCA
GTCAGTAAGCATTCTGAGAGCGCCTCCTGCAGGTCCTGCAGGTAATGACAAGCTCCTGG
G
CACACACATGCTCAGGGAGCGCTCTGTAGCCTGGAGAGAGCCAGCTGACACTGCTACCT
T
ATCCTCAACATGGCCCCCAGACAGCAGTCAGTCAGTCAGTCAGTCAGTCAGTCAACAAA
C
ACAGATCAAGGACAACTATAGCAGTCTCTGGACACAGCCCAGAGAGAGGGGCATTGCG
CC
TGGACAGGTGCCCTGGGGCTGGCAAGGCGGTTCAGTGAGGCTAGGGATGAGTTTGATC
CC
TGGAACCAATAAAAAGAAAGGAGAGAAGCGACTCCACAGAGTTGTCGCCTGACGTCCACA
TGCCTGCGAGTCATGTTGGGGACACACACACACACAACTCTAATAATTTTAAAATGATCT
TTTAAGCCCAGCATGCTGGCACATGCTTTTAGTTCCAGTATTCAGGAGGCAGAGGCAGAC
AGAGGTCTATGAGTTCAAAGCTAGCCCGGTCTAAATAGTGACTACTAGCTAATGCTCCAT
AGAGAAACGTTCTCTCAAAGCCAAAAGGCGGTTAAAAGAGAACAAAGGGAGTTGGCGCCT
GGAGTGAGGGGTCCTGGGGGGAGTGGACACAGAGATGTTCAGGGAAACTGGAGATTAAAG
AGTGATAGCCAAAGTGTGGTTGACAGTGTGACCTGATAGGCCTCGGACTCCAAATTACAC
AAAGTAAGAAAGGGCAGGAGTCGAAGTAAGAAAGGGCAGGAGTGGTGGAGTGGGTGGGGA
GCGGGTGGGGGACTTTTGGGATAGCATTGGAAATGTAAATGAAATAAATACCTAATAAAT
AAAGTAAAGAAAAAGAAAGGGCAGGAGTGGTGGGCAAGCGGCTAGGGTGGAGGGGTGCTG
TTAGGGTGGGATGCCGTGAGCAGAGGTGTGGGCCCCCCCAGGTGATGTGAAGTGTGCACA
AAGGTCCTGGGGCTTCAGGGGCCGTGGCCTGGTCCAGGAAGCACAAGCAGAGGAGGTG
AG
CAGCAATTGCAGGTGAGCACAGGCAGGCTTGCCAGTTATCAGCAGGGGATGGCTTTGA
TC
TTCACACAGTAGGCAGTGGGAAGCCATCGTGGGCTTTGAACTGAGAGAGACACTGGGG
AT
GCACCTTCCAAGTTTCTGTGGGGAAGGCTGAGCTGGGGGAATGCCTGCCATGCCCAGG
TG
AAGGGCGTGAGGAGCGGGGCATGAGCTCTGCCAGGCTGACTGGAAGCCTCCCCCGGCA
GG
CCCTACTTCACCTACTGGCTGACGTTCGTTCACATCATCATCACCTTGCTGGTGATCTGC
ACCTATGGCATCGCACCTGTGGGCTTTGCCCAGCACGTTACCACCCAGCTGGTGAGTAG
G
GTTCCTCCTGGGGGGTCCCCGGCCTCTCCCAAGGAGCTTTGGCACAGTTGGCACCAAGTA
TCTCCCACCACAGTACCCTGGCCCAAGTTGGAGATGCCTGAGGCTCATAGCCTCTTTCTA
GAGGCCCTTTTCTGGGGATGCCCCCACCCCCGTCCCTTTCTCTTCTCACGCCGAGGGCTC
TGGCTCCTCTAATGCCACAAACTACCTTCTTGATAGGTGCTGAAGAACAGAGGCGTGTAT
GAGAGCGTGAAGTACATCCAGCAGGAGAACTTCTGGATTGGCCCCAGCTCGGTGAGGCC
C
AGGATGCCCGGGAGCCCTGTATCCTGCCACTCCACACTGGGCAGAGGGGTGGATGGTAGG
GTGCCCCGCCCACTGCTTCCGAGTATGGGGCACTGGCTCACAGTCCCCCCCCCCCCCACT
CCAGATTGACCTCATTCACCTGGGAGCAAAGTTCTCGCCCTGCATCCGGAAGGACCAGC
A
AATTGAGCAGCTGGTACGGAGGGAGCGCGACATTGAGCGCACCTCTGGCTGCTGTGTC
CA
GAATGACCGCTCGGGCTGCATCCAGACCCTGAAGAAGGACTGCTCGGTGGGTCCTGCCC
C
TACCCCTGCACCTGCCCCTGCCCCTGCCAGCCTCCTTCCTTCCCCGGCCCAAAGCTGGGC
TTGGAAAGCTGGACAGTAGTTCCTAGGAAGTACCCAAGTCCTGGGGAAATGGGAAAAGCC
CTTGTCCTGCGGGGTTCGCTGCCCACACCATCTGACTGACACGGCCTGACCACCTGTCCA
CCCTCTAGGAGACTTTAGCCACGTTCGTAAAGTGGCAGAATGATACTGGGCCCTCAGAC
A
AGTCTGACCTGAGCCAGAAGCAGCCATCGGCGGTTGTGTGCCACCAAGACCCCAGGTAC
A
GCCAAGAGCCTCCTATGGGTCCAGAGTGGCACCCAGCTGTTGGAGCTGCCATCTGTGATG
CTGGTGGGTGGGTGTGGCAGGCTGGGACATCCCAGCCTTTTATTTTGCTCAATCTGACCC
CTGCTTCTAGGACCTGTGAAGAGCCTGCCTCCAGTGGGGCCCACATCTGGCCTGATGAC
A
TTACCAAGTGGCCGGTGAGTAGGAGATCCATGGAGAAAGGTCTGGGATGAGGGTGGGGAC
AGCTGGCTTTCCGTCATGAGGCCCACTGCCATTGGTCCCCTGTCTCTTAGATCTGCACAG
AGCAGGCTCAGAGCAACCACACGGGCTTGTTGCACATAGACTGTAAGATCAAAGGCCG
CC
CCTGCTGCATCGGCACCAAGGGCAGGTGAGCCGGTGCCTCCAACCAACCCCTGCAGGCTG
ATGGACCTCTGTGACTGTCTTCTGTTCTCTGCTCTTGGGTGATGGGGGGAGGCAGGGATT
GAGGGGCAGTGATATAGAAAGGAGCTTGTCCCATCCTGCCTGCCATGCCCAGGGACTGGG
CTCCCCAGCTGATGCTCTTTAGAACGCTGACAGCCGTCTGCTICCTGTCCACAGCTGCGA
GATCACCACTCGGGAGTACTGTGAGTTCATGCATGGCTATTTCCATGAAGACGCGACGC
T
GTGTTCCCAGGTAGTGGAAGCTGTAGGGATTCTGAGGGCCACTGGGGTCCTTACCCMG
GAAGCCTCAGACTTGGCTTGCCTCCCAGGGCAGAACTACTGACCCTGTGTCTCCCACCCA
GGTGCACTGTTTAGACAAGGTGTGTGGGCTCCTGCCTTTCCTCAACCCTGAGGTCCCTG
A
CCAGTTCTACCGGATCTGGCTGTCTTTATTCCTGCATGCTGGGTAAGAGGCACCCTGTT
G
CCCCATGCTCAGACTCCCATGTCTCCCCTCTTGGGTGCCGGAGAAAAGGGCTTCCAGAC
C
GAGCACACTGGCTCAACCTGCTAGTGCTAGACTGCGCTGTGGTGTGCTCTGCGGGTGAC
C
ATGGGCACACAGGAAAGGCTGATGGTGCTCATGTGCCTACCCCAGCATAGTGCACTGCC
T
TGTGTCTGTGGTCTTCCAAATGACCATCCTGAGGGACCTAGAGAAGCTGGCCGGCTGGC
A
CCGCATCTCCATCATCTTCATCCTTAGTGGCATTACAGGCAACCTGGCCAGCGCCATCT
T
CCTCCCCTACCGGGCAGAGGTACAAACTTGGGAGACAAGGGCAGAGAGGGTGGGATGAGC
CCTTCCTTTTGGATCTAAAGCTTTATAACATATGGGGAGGACCATTGTAGCCTGTGGGGA
GGACCATTGTGGCTTGTCAGGAGGACTATTGTGGCCTGTGGGGAGGATCACTGTGGCCTA
TGGGAAGGACCAAACCTGCTGCTTCTTGCTCTGGTTCCACCCCAGAATCTGCAACAAGGG
CAGAGTCCCTGTTGTGTCAAGCTTACCTATAGATGGGCAGCTAAGGTAGAACCTATTGAT
TAGCCTCTTAATTCATGACAGGAGGGAACAGAGATACTCTTGAGTCCCCAGAGATCCTTG
CTCCTTGTTCTGTGAAACCCTACATTTGGCTCCTCTCCACCCTCAGGAGGAGAGGTCTTG
AGTCTGTTGCTCCTTCTGGCCTGCGATCTCTCCACTGCCTGTAGTCTCCTAGGACAGGCT
GGCTTGTGCTAAGCACGGGGTTAGAGCACACCCAGGTTTGCTGCAGGGTTGGACAGAGCA
GGGCCAGCAGCTCCTGTGGCATCCTCCGAGTGGGAGATGTGCCCCACAGCTGGTACCTGG
CACCCAGCATCAGTGGCGACATCTCTCCTCCCTGACCCCAGGTGGGCCCAGCCGGGTCGC
AGTTCGGCCTCCTCGCCTGCCTCTTCGTGGAGCTGTTCCAGAGCTGGCAGCTGTTGGAG
C
GGCCGTGGAAGGCCTTCTTCAACCTGTCGGCCATTGTGCTTTTCCTCTTCATCTGTGGC
C
TCCTGCCCTGGATAGACAACATCGCCCACATCTTCGGGTTCCTCAGCGGCATGCTTCTG
G
CCTTCGCCTTCCTGCCTTACATTACCTTCGGCACCAGCGACAAGTACCGCAAGCGAGCC
C
TCATCCTCGTGTCGCTGCTGGTCTTTGCTGGGCTCTTTGCTTCCCTGGTGCTGTGGCTG
T
ACATCTACCCCATCAACTGGCCCTGGATCGAGTACCTCACCTGCTTTCCCTTCACCAGC
C
GCTTCTGTGAGAAGTACGAGCTAGACCAGGTGCTACACTAACTGCAGAGATTGTGTGTC
T
GCCCTGGGCCGTGTGTCTATGAACCGGTGGGGCCCTGAGCCCAGCAGCTCGGTCCACA
CT
CAAGGCTGACTCCAGATGAGACGGGCGGTAAAGGCAGGCTCCCCAGGGAGATGACTCC
TC
CTTTCTCAGGTCTGAATGTTCCTGACCCAAGTCTGGGGGACATCCAGGACACTTGCTTC
T
CTGAGGCTCAGGTCCCAGGCCCTGCCTGCCTCTCTGGCTCTATAAAGATGATAACTTTT
C
TTGGGCCTCTGGCCTCTGCGGGTGCTGTCTCCCCACTGACACTGTGACTGTGACCTCGC
C
AGACACACAGCTGCTCTCTCAGTTGTCCCCAGGGTTGAGGTCCTTACCATGCTGCTATG
A
CCCCGTTTTCCTGTTTCTCCTCTCCTCCCTGTCCTTCCTGTGTTTGTCCGTGGACTCGTG
AGCCTGCTCCTGAGGCTCCTGGACATAGAGCATTGTGGGGAAGGCTCTGGCTGTTGTCT
A
TGGGGGATGACAAGCAAGGAGAGATGGCTATACAGGGATGCTAGGGGCTTTTGTTAAG
CA
AAGAAGCCAGCTCTCCTAAGCCCATCAGCTGCCCTAGCTCCAGCATGTGTCTGGCTGCA
C
AGGTTGGCTCTGATCCCAGGATGCCCCTGCCACCTGCCCTCACTCCTGCGTGGCCGTGG
G
CCGAGCCGCCTTGAGGACTAGCTCCCAGAGGAGGCCTGAGGCCCAGACTGGTGGGTTT
TT
TGGTTTTGTTTTGTTTTTTTTTTTCCAATTTATATTATGGTCCTAATTTTGTAAAGTAAC
GCTAACTTTGTACGGATGATGTCTCAAGTTTATTAAATGACATTCTTTATTAAAATGCTG
CCTTTTGCCTTAGACCTCCAAGAGAAGGAAAGCTAGAACTTGGGGCTACAGAGATGGCTC
ACTTCCTATAGAGAACCTGGGTTCAGTTCAGAGATCCGCCTGCCTCTGCCTCCCAAGTAC
TGGGATTAAAGGTGTGCGCTACCACCGCCCCAGCCAAAACAATGTTTTTATTTGAAAGAG
AAAGCCCCGGGGCCTTGAGGCTGAAGCAGACCGGAACACTTGGGATCTCTGTCATTTAGT
TGAACTAGAAACCAGATTGAGATCTCAGCAGAGCCCCCAAGGACCCTGAGAGAACTGGGT
TACGTGTAGAGCCCTGAGACCTCAACCCCAGCTGCTCTGCCTTTGCTCCAGGGACATCAG
GGGCTGATGGGCAGCAGGAGTGGCCTGTTCCAGCAGAGGTGGACCAGCGGGGAGGAGGCC
ACGTGTTTGCTCCAGTCAGCTCGAAGAAGACCCTACACACACCCATGAGGCACAGCCACT
GGGGACATAGCTACTTTATTCGTGGGCAGAGGTGCCAGTCCTGTGGCTGGTGGGGGAACC
AGGGAGGCAGGGGGGTGGGCCGGCACATTGGTGGCTGACTGCAGTTTGGTGTGGC
Mouse iRhom1 protein sequence full-length
SEQ ID NO: 3
MSEARRDSTSSLQRKKPPWLKLDIPAAVPPAAEEPSFLQPIIRRQAFLRSVSMPAE
TARVPSPHHEPRRLVLQRQTSITQTIRRGTADWFGVSKDSDSTQKWQRKSIRHCS
QRYGKLKPQVIRELDLPSQDNVSLTSTETPPPLYVGPCQLGMQKIIDPLARGRAF
RMADDTADGLSAPHTPVTPGAASLCSFSSSRSGFNRLPRRRKRESVAKMSFRAA
AALVKGRSIRDGTLRRGQRRSFTPASFLEEDMVDFPDELDTSFFAREGVLHEEMS
TYPDEVFESPSEAALKDWEKAPDQADLTGGALDRSELERSHLMLPLERGWRKQ
KEGGPLAPQPKVRLRQEVVSAAGPRRGQRIAVPVRKLFAREKRPYGLGMVGRL
TNRTYRKRIDSYVKRQIEDMDDHRPFFTYWLTFVHSLVTILAVCIYGIAPVGFSQ
HETVDSVLRKRGVYENVKYVQQENFWIGPSSEALIHLGAKFSPCMRQDPQVHSF
ILAAREREKHSACCVRNDRSGCVQTSKEECSSTLAVWVKWPVHPSAPDLAGNK
RQFGSVCHQDPRVCDEPSSEDPHEWPEDITKWPICTKSSAGNHTNHPHMDCVIT
GRPCCIGTKGRCEITSREYCDFMRGYFHEEATLCSQVHCMDDVCGLLPFLNPEV
PDQFYRLWLSLELHAGILHCLVSVCFQMTVLRDLEKLAGWHRIAIIYLLSGITGN
LASAIFLPYRAEVGPAGSQFGILACLFVELFQSWQILARPWRAFFKLLAVVLFLF
AFGLLPWIDNFAHISGFVSGLFLSFAFLPYISFGKFDLYRKRCQIIIFQVVFLGLLA
GLVVLFYFYPVRCEWCEFLTCIPFTDKFCEKYELDAQLH

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A genetically modified NOD.Cg-Prkdc^{scid} Il2rg^{tm1Wjl}/SzJ (NSG) mouse,
wherein the genome of the mouse comprises a mutated Rhbdf2 gene such that the mouse expresses mutant iRhom2 protein which differs from wild-type mouse iRhom2 protein due to one or more mutations in the N-terminal region of iRhom2 selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein.

2. The genetically modified NSG mouse of claim 1, further comprising a xenogeneic tumor cell.

3. The genetically modified NSG mouse of claim 2, wherein the xenogeneic tumor cell is obtained from a tumor of a human subject.

4. The genetically modified NSG mouse of claim 2, wherein the xenogeneic tumor cell is a tumor cell obtained from a breast tumor of a human subject.

5. The genetically modified NSG mouse of claim 1, wherein the mouse is a NOD.Cg-Prkdc^{scid}Il2rg^{tm1Wjl}Rhbdf^{P159L}/SzJ mouse.

6. A method for producing a mouse model system for assessment of xenogeneic tumor cells, comprising:
providing a genetically modified NSG mouse, wherein the genome of the mouse comprises a mutated Rhbdf2 gene such that the mouse expresses mutant iRhom2 protein which differs from wild-type mouse iRhom2 protein due to one or more mutations in the N-terminal region of iRhom2 selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein;
providing a xenogeneic tumor cell; and
administering the xenogeneic tumor cell to the genetically modified NSG mouse, thereby producing a mouse model system for assessment of xenogeneic tumor cells.

7. The method of claim 6, wherein the xenogeneic tumor cell is obtained from a tumor of a human subject.

8. The method of claim 6, wherein the xenogeneic tumor cell is a tumor cell obtained from a breast tumor of a human subject.

9. The method of claim 5, wherein the mouse is a NOD.Cg-Prkdc^{scid}Il2rg^{tm1Wjl}Rhbdf^{P159L}/SzJ mouse.

10. A method for identifying an anti-tumor activity of a test substance, comprising:
providing a genetically modified NSG mouse, wherein the genome of the mouse comprises a mutated Rhbdf2 gene such that the mouse expresses mutant iRhom2 protein which differs from wild-type mouse iRhom2 protein due to one or more mutations in the N-terminal region of iRhom2 selected from the group consisting of: p.I156T, p.D158N and p.P159L, wherein the genetically modified NSG mouse is characterized by hairless phenotype and increased growth of a xenogeneic tumor compared to a mouse of the same genetic background which expresses wild-type iRhom2 protein;
providing a xenogeneic tumor cell;
administering the xenogeneic tumor cell to the genetically modified NSG mouse, producing a genetically modified NSG mouse comprising a xenogeneic tumor cell;
administering a test substance to the genetically modified NSG mouse comprising a xenogeneic tumor cell;
assaying a response of the xenogeneic tumor cell to the test substance following administration of the test substance to the genetically modified NSG mouse comprising the xenogeneic tumor cell; and
comparing the response to a standard to determine the effect of the test substance on the xenogeneic tumor cell, wherein an inhibitory effect of the test substance on the xenogeneic tumor cell identifies the test substance as having anti-tumor activity.

11. The method of claim 10, wherein the xenogeneic tumor cell is obtained from a tumor of a human subject.

12. The method of claim 10, wherein the xenogeneic tumor cell is a tumor cell obtained from a breast tumor of a human subject.

13. The method of claim 10, wherein the mouse is a NOD.Cg-Prkdc^{scid}Il2rg^{tm1Wjl}Rhbdf^{P159L}/SzJ mouse.

14. The method of claim 10, wherein the test substance is an antibody.

15. The method of claim 10, wherein the test substance is an anti-cancer agent.


SEQUENCE LISTING (9 sequences)

SEQ ID NO: 1 - protein, 827 aa, Mus musculus
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKIVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 2 - DNA, 30055 bp, Mus musculus
acagaagcaggcagatctctgagttcaaggatagtctggtctacagggcaagttccaagactagacagagaaaccctgtatggaaaaacaaacaaacaaacaaaaaagttgcaggccagctgggcgtgctggttcacatctttaacctcagctcccaggaggcagaggcagagacaggtgaatctctgtgaatttgagaccagtctgggctagatagtaaattccaggccagccaaggctacatagtgaaactttgtctcaagacaagataagggaataaaaaaatattcaagccagtctgctttacagagcaaagtactgtctcaattttttcagtatttatttcaattttttacagttttttttaagttataaaaacatagcaagtgtatgcattccactttttgtttttctactcaagggcattttggagataattccctatcaacacacagttacctcattgcttttaacagctgcaatgtgacattcccacacgttacacccaactgtttctcactctcccacaccaggcaggagaggtggtttactgcagccaagtctgtgatctctccatctccagggctagggcggcatggcacaagtctctgatgtcaccgaagcatcatgcatattctcgtccttggccgtacaggggtgaggacacagcccagccgtccagcagtgaggacacagcccaaccgtccagcagtgaggacacagcccagcccagcccagcccaggtgccagagtagcatctgccaggctgagaggtggattgggcctgaatggttccagcagccccacggtgcctcttctgcctgatgctctttgctgccatgggacagacagaatgatcctcacatgatggaactcacacgcatgccaggcaagtccagcctcccacctgcaccccaacccccagggctggttcttttgccgctctcagcatggtttcctaaggatatgtggaggggtcactaaatggctccctgcctgtgcttcagagaaacagctcccaagtttggggttattatctgtctttgccccagccttgcctttccgtggctggagactgggaaggagaagtttgccaccctgcctaataggaagtaactaaagctctaagcatgtggtgcacacacattttatttatttatttttacatgaattagtgttttgcctgtgtcatatatgtatgtatgtatgtatgtatgtatgtgttaagtatgtattatgatgtacatgcctgtggaggtataagaagggcatttgatctctgaaactgagttaaggctgtctgcaagatcccaagtgcatgctgggaactgaacctgggtctctgcaacatcagcaaaagcttccaaccatataattatttcccctgccccacttttttttttttttttttaagatttatttattgccgggcagtggtggcacacacctttaatcccagcacttgggaggcaggaggatttctgagttggaggccagcctggtctacagagtgagtttcaggacagccagggctacacagagaaacactatcttgaaaataaaaaaataaaataaattttaaaaattcttttatttatatgagtccactatagctgtcagacacaccagaagagggtatcagatcccattacagatggttgtgagccaccatgtggttgctgggagttgaactcaggacctctggaagagcagtcagtgcttttaacctctgctctacccaacccaccaacgtggcagactgggggcagggtggttaagagcaacaagagcccagagacctggctcacctctgaaagcagctctgctgcagcgccccctggtggtggtctctccatactctctggctgggcgaggacttcagaaagaaagactgaggccattggtgcaggggctgaggatagggactccagacctgggggtacaggtctagttcgttcctctgccatttcctgcctgccgtaagtttccacatcaaatcccaagtgaggggctaacccaggccctaggcatctgtatcagtggcaccccctgcccttctccggcctgctgttcttgttcaggagctgacaggtccggcgagtgctcgtaggtgggagcatgggagtgttggacagggtgtcataaatgtaggccttcgtacagggctaggtacgcgaacatgaagagtggtactctaccaggaagtgggtaagaacatcacaagatggcaccacccagagccgagttaagggagggatatctgggtccaggagggagatgaggaggcacagcccagctcctatgggctcaaggtggctagagacgtgggctaaggaggataaaagcctgtggcttaaacttgagggagggccggccgtcaccactactaataatagcaaagataacagcagctgctagttagagccaggggccccacaaatgctccctgttactgctaccacacagagaggggaaagctgagaccgaggaggcttaggggattcatctaagaccacaggagcagtcaatggcagatcaggatttgaacccccggctctgttagctggagtcatatatagttatttctcattacagccaacaaagcaagttacttggtcagtattggccaggctagagtatccaaagcctgggcctgggggctgttcatgacacggagatgtggagggccttcctctgcatctattgccagttactgtgagagccagctttcagccttagcaagaagcctctgtgtccttgggtgcagagcaacattcacagtttcctaagggacagtcccaagaactagcatatacctcggttgcctttccaactgctctgtgtttagtcctggggtggttaaggggacaagacccaacttccagcaaggacccggtctggcctagaagggatgccaggcctgaggagagatcattctaatggacgaaggagagacagcagctagagaaggccaaggcttcctagacgatgtagctgcagcgatcgatcggggattctggaaaggatgcaagcctagtcgaggctcctggagtcaaaggagcctgaggtcacacgagaaagagaaggggaaattgagtggtttttgcttggttgttatgggggtgtgtccgtgtgagcgcgtgtgttgctggagatcgaacctaagatctgtgtgctaggcaagtgtttaaatgtggccataagtggagcagcaggagctacaagcccaggcagaagccgcgggccgaccacgccccccgaagcaccgcccacacaccaagaagccccgcctcaatgaggccccgcccacacccagtccccgcccctgcgccctcgcgcaggtagggaagaggcggagcgctggcgctcagccttgtagccgccgccccgccgctgcccactctgctctcagccgcttcccgggacgtggggcctccgagaggtgagcacggggaattgggtgcggcggagctcgggtccgctaggccgcgggtggccagggattcacggggctgccccgttcggccgcggggaaggtcgggggctgtgcgccccgcagagcgccctagaggccgaggctggactctgtgcccgcgggaccgctggatccctcccgcagatccttggcctctgctgggaccaggacgcctaaaggggttccccggggcacagtcaccagatgtctgggcgcgtggtttgcgcaaaagtttaaaagcccagagaggaagagagggcactgcccagtgttggaacatgcgactccgcctccaaccagaaatcccttttagaccttaagactatattcccctgtctcccgggtgaacttccaaagtcctcgggcagcgttttgtgcgtggagcttcgccgccgtggtagtaacaggtgcgggggtgggggatggggaaggctcacaccgccagagtagtccgcggctcagaaagtgtactcaggagtcctggcttaaggaccgaggggtttggagagttgggcccccagctgatgtttctgcattggattgaaagttagggagcgaaaggtctgtagggcccaggtctctaccacaaccccagggcagaagggaagccaggtccataccttgattcaactccaggaaacaccgagccgcgagtctgtagggccgggacatagagagcgaaggtgaggtgtacctgagggattgcctcataggtggagcggttgctgtttctcaaccagctgcattgggggttccagtgtgggtgacatcttgtggtgaaatactgtccccagcatctatgttgtgctgttgtgattgtagtcagggagaagaaaatgaaacttggtttcaagcagtggttctcagcttaggggtgcttttgcccgctaaggatatttgacaatccctggagacacttggccatcacaggtctgccccacagtacagaatctggcccctcaaaagtcaacattgagtgaccgtcctatgaccaccagcccactagggtacttcttgtgaatttctctccctacctaagtttctccaggggctgggaagggccaggacgaatctcagtgagagataaagagataggtgggcaggcttcgccctggctggccactggccctgtggaggagaagctgggaacagtggctctctcgaagcacaggtctgtagtactttacccaggatagcttcagacacaggataagctcaaggtaagccagagtagccctggggatggaggaagggcaggaccaagctctgttcccatggagtttcccaaagctggatgagctgaggtctcccggatagcggaatgccatgtgaccaactggaattttccttctgaacacagaactgactcccctagctatttacaccaggaaagtaacatccaaggaataacgggtccactgattccctgtggctcctctctttccttccaacaggatgggttgcccctggggcggtaggaatcctggtcagggtgagctcaggccctgctgtagtatttgctgagtgacagtaaaggatggaggctggtaaagagctttccacccacggggtcccccaaaaccgcggagttgggctcctgggctgtactcttagctttctgggaatggaggtgaggctgttgctggctgggtaggtcagggccagaatcctctcttccggcaactaacatttccatctccctttgtcctgtctagttttggacacttcttgcttgagagccctggtggggtgagaagggagtggtgggactgggggcggggcaggagtctcgggttggttggttggttgattgattttggtctttttaaccataaaaccctgatgtgtagctagacttggtggtgtgtgctttgatgtcccagcattggcaggtagtggcaggagagtcaagagtttgtcaccctcagtaacagagtgagtttgaggccagtctgggctctacaagaccctgtctcagaaacccagcagaagaccagacctttacaatgtagcagttaccactactgtatatggctctgaagtcataaagttaaacacccagacctcctcaaagccacctaccccaaacctgtaactctagcttcactcgctcattctggtcagtgcccacccccacccccaccctttctatacacgctgctgccaggggaggggaggagacttccaaaagagcaggggtaaatcaccccagactggagcaggacgaccactgggggctcaggcctactctgggctcactgatgtttttcttgtgacctgagctctggacagtgccctccttggttgtgtgtctgttcagcttgtgtgtgcgggatgcttgctaacccccccccccccaatctggaattacagacagttgccagctgccctggaaatgctaggaaccagtctgggccctttagaagagcacccggtgctattaactgctgagccatctctctagctccatgacttgtataaactgtgtcccagacaggcaccagatgacagcaggaagatgtcaaggggccggcaagtgttcatgtttgtgtttgggtaacttgatgctggcttctgggcctggatccccttacacagcatgtgggaggtgtgttctccctgccccaatccagcatgttccttggaactcatgggatcctgcctagatattgccccattgctcaaggggatttccaaagtgacctcaccatctgtgccgttgggagcagctcctagtttcctatccagctcagacaggctgggggaggagtgctggctgacctcagcagccagcatggccctgggacagggacgctgccaggccgggcagctcttggcacacaggcaacctctcaagaggagccagccacgcctgccccgttgttgggaggaagggactgccccaaagtgtccctggccttcccaggcagcagaccagcataagggaaatctcttctcatcttcagagagctatggagagtcactgggcaccatgctcccttcaccagatttattcagggacccagatgtaagcatttgggtttcagcagctgctgaaagggactgtcagcttcactgtcctggctccccgcttagtggtttgagccaagtgagttctggcagggtgtgggatgatagacaccatggttggctagaggggcaggtgattccgcgctcaaggcccaggagggacggtcctggggccagcagacttgagtctgcaagaagggtgggggtcacttgagtaggcttgcctcagtttctacatagctgtgtagtggagtcatttaagtagactaaccttggtttctccactataccgcggaggattgaggtggcctgtggacatgtgggtgtgactggtggagatactgaaatgagccatcattgcagcaagaccgctgtgcctggcacaggcttgggtcatgggagaaaccctgcctcgtaggctgctgggatatcagaacagaaagcatgtcagggtgatctgaactctacaaggagcagtttctgcggtagagaaccccctggggctgggagtgagcgttttattgcaaagccctgctcccctgcctcagtaggtggccactgaacccatgtccacagtgtcacggtgatgtaggggagctacccgcccctggtccctggcacagggtgccttttccctctccaggtgggtccctgaagggtgcagaacctgttgtggtattctgggtagacacacgactgaaaactgagggctcagctgggcccttagggactttatctgtcaggctgcataatgacagtggcctccgccctccctcagttaatgagataatgcttgtgaaagctctctgatagctgagtggctacaccggcagtaggtgttgccactcctcgatcctcgtggatgctagaggaaagctttctggagccgaaggcatttgcaccccaactacacagatggagcagccactctagacacgcccttttgccgcttggcttttcatgtttcaagaggggtaggtttaagaactagtggagagccggctcaggggctgagagcacactagctgctcttgcagaggacttggattcaattcccagcaaccacatgctggtttgcaaacatctgacattctgtttccagggtacccaacactctcccctggcctccacggacactgtacgtggggtatagaaatacacacagataaaaatacctgtacacacagagaaaaaaaaaataaaagtaattctttaaaatataaaaaaaacctcaggacccaagcaggctacagccccagcgcctccggaggtagcgcagagactgaacatcagtgagccttgatttataactcaaactgtcagggagaatcttgtcacgtggctcttggctcgtaggctgagtgggtgggtcatggtgccaagggggaaaagccattagttagctactgctggtgcacccggcattgtacatgcaggatctgcctgccatccatgtagtgactgtcaccccatctcacagaggaaaccgtgacagaccacacgtgagccttgactctgactcccagcatgccatgagagtcctacactgccccaaagtagcagatagcattgaaggttcacctgctgtgggcacagcttggatggccctgtgtccaagttcccttggcacccactgtgaccgggaccagccgcttcctggggaagtagggtgccccagggcagagttctggaaaatcaagtttattagcttcttaatgacggtttacaagccaaaaagtgtttagctggcgtgttcccttcctgctgagaggcatcaaccctgcagaaggagatctgggcaggatgccgagcgccctgtcctgagtatgctgaggaaaacatctcagggcaacctgcaatcctttggtgctttagagcgctcatacctgacctggacactgcctgtttctggctgaaggacccacactatgcaaggactctgtaggatagggtgagactttgtccctcagtggaacatgctgtatgatagatgagccctagttattggtgacagaaacacccaggtctcaagagcccacataaacaacacattgagttagaggtattaagtagcttgctcacaatcacacagtaaagctggccccgtgaccctgcttctcctagtcaggcagttcctatctctgtagccacgctccaagatggcggatggaaattgggtgggcgattggcttgccagtatcaggcgaaaccccagctgtgtggtgtgtcacatcttggtgtcagggaagcctagagaaataaaatcaccacgttactcagccttccctgcacccaccccctcccaggctacaccgcatcctgccctgaccactgggactgtactcttctggttttgaagactgatgaaaacccaattcccagtagccaatgttcctatgacaggcagagcttccatatgaaagcaagctcatgttgatgtggtgcccagggtaaaccgctccctctgtgggagcctcctttgacctggcgtcttgacccccagggaggggcagcagaagtgggacagaatatatagtcttctctggagaccagacggctagacaagcagcctagaacccagccgcctattggctgtctctgtactgttccctaaagctgtggtcagcatcccagcctgcagccctgccacatgacctgctgttgctgcctgaggtgtgagagggctcagttctactcagagcacctgcatctctgtgactggaggggacatgcctcgccagcagcttctagagtcatgaagtttccagaggagctttgagctgcagctcctagctgcatacccatttcccagtatgttagcatcttgtgtgtgtgtgtgcgtgtgcgtgtgcgtgtgtgtgtgtatggaatccaaagccttacgcatgccgagccagctttctgctactgagctacacccatgcccaattgttgtatcttgagctctggggatacagaacctgtcccctgcctcaactgctcctaggacaagagtccccagcagaggcggaaggcatttgcacctctgctgatctggctggccagtagtccagagaaaggccagatagatggggagagggggaacccgggctggacctctctaagagcagggtggcacacccacacagacaagctcacatatgctctgctcttggcagctccctggaacctcgtgcccacttcccgtgcctgtggtgggccgtgatgcccaagctgctctagttgggtcagagggaaccgagagcgtagggtctgaatgtggaatacccccaaagaagccagcaggctgccaagtccttgagcctgctcctgtaggtctggtcttgtgaaataagtttggcacaagccctgggaagaaggggagaatgaagtgagggctctgtgggggaggcctgttgcttgtgaggtctgtgcagcgtgtgaacccacaagccagttttcttttcttgtgggtggccaccaggcctgtgcaggccagcagagcgtggtgccttggcatacataaggctttggattccatacagccatacacacacacacacacacacacacacacacacacacacacacgcgcgccacatttgtgctaccacctttggcccatacctctgaggaaaagcacttcaggagcacagagccctctgtcctttctccgtggtccttgcagcttccattcgatgcatcctgctgctagaccgcaacccccccttctgtattttgtgaccttttctgggtcctggccccagacctggcctcttcacctctctgcctttggctccatcattcctgcctctttccagggccaggagatgctctgcagtgagtcaccaatttggagacttcttagatgacagtctgtcttacatgggttagccttccacggccaccagggctgtcaagctttgggaggccagcaacagaaactaactttctttctttctttctttctttctttctttctttctttctttctttctttctttctttctttctttctttcttagcgggatttcaacagttgagggttccacttcctctcttcagctttcgaaaaactcagaggttcccagcagggccacaggagggaagatgcagttttggtttctgtggcagggaaaccggggagagaagttgcttgcttgcccccagcccacactgtcatcatcctgtgactcagcaaggacatggcatatggtctgtctgccaccacccccttctctgcccgctctctgcatgtcccaggacgtggctgagtgaggcttctggggcaggtgtagtttattaagccctctgtgactcgtaacacccccctttggccatatccagttgtgaataatggtcttccccctttccatggaggggagagggctacccacttcttgtccattggtccctcctgctgcccacatttctggcccatatgtgaccttggctattacatcaggcgcctgcttgtttgggaatacaggctccctcaatgtcctgtttcctcaagtctacagagcttacgggtgtagggaatcattaataacgctgggcttccaggatagtgggggtgcgtcaggtcagacctgtgggtacaggtctaccccttcttggagcagagagggaggaatctaagcatatgccacctgcatgggaagggagggcaccctgcctagagtggttgggggagcaccccctgggggtgaccttgcctaccatgttatcatgcctggattggacctgcctcatttgggagtggctgggccccaccctttgtggggaaaccggattctgtgggacattctaggtagtctcagagcctttgtttctgtgtgcctaatctgggccaccactctgtgggtggtgctatccggaaggctctctggagagcacgctgacccctagtggaggccatctaccaagggctcctggggcggggggtgggggtgagggaggcggggggtgggggggaaatctcagctttgcactgcattcttctgctgtctgccccacccccacctcttggtagacaccaccctagaggaagaggaaacactgacacccctccgccctgggttccgggtaaatattcttgtgagaaagacttctccctagaagttagaccaggatccaacttgagcaggtggcagagaccataatcctggctttggtccctgtcactcccacccatgggccagctgggcagcaagtctagggtcagagagctcggcggatatgactgtgggcaagaggaagcttgctcccaggatgtcacaatttcctttgcaatgagcaagcacctctgggggcgggagataagtggcgggcgcggagggcggctcctgcaatttttttttttttttggtctagtttctgtggcctttcatagacgtaggtcacacggggggactagagcaaatctgatcattcccaccactgtgccaggcagctgccagcccgcccagtccagagaagggttggactttaccccccccccccaaggttggggcagaggggtcaggagaagttctgggagaaagtatcagagaaaaaccttcattatccaggctgtctgtggtcagcaggtctgagccatcaccttgtccaactgccagtggacaggaagggaaactgaggcacagggatgtagcttagggaccatcattcaaagctcggtactggcttcgcagatggtggtagagagtttcctggcacgtgactccttacagtctctgacccacccctgcggtcggacttctttctaggggaccctgagattgtcccaaacttcccctgtcctgtggccttaagtcatgtctgccttttccatgtggagattggttaggggggtggggtgggagggatcctgggtatatttgggggaggggttgtcaccagctggaatggcctgggctctaggggcagctcctcagaggggtaagagtgtttgtggacaaccagggaactaggccaccaggcctcatcctcagccagcacccagagacctccatcccagccctgcagagggactgggcaaggtgagtgacttccagagccaagttcagagtttgagagacagcctcagcccagcatccatgcagtccgtccccagctgcacagactcctgccctcaccagactcatccagctaggacttgatgggcagtgattgggtgggagattagagtaaggggctcctgctgtcaggagctgtgggatgcagcgtgagacagagcagggtggtgagaggcttcctggaggtgacacgccttagaagcgcagtaaatacaaaggttgctcccagggcactggcaaggcagctgatgcggaaggcagggtggcatgtcacagcccggagcctgcacaggtcctacccctgacacactgccagccgcatgatcacctagactctgagcaacaagaagacaccagagatgaacaatggttcattttctgggttaaggaaagacctctgaggtccatggtctgtgctgccgccagagaccattgttacagtctgccgtccatgttatctatgaccattgctgtcaccagaggccatgtggatgcatgtgaatgtccctgatccctggccaagcttctgtgggcagaaaagcttcctctgcagtgatattaatgtctgtagagtcataactgagagtgagcgacattgaggggcttctgtgacaaccgccccccacccctgagagagagagagagagggagagagagagagagagaaaggagctattgaagagagtctgtaaaaattgtccaggcagtggagcctttaatcccagcactggggaggctgagccaggtggatctctggtgtgtgtgtgtgtgtgtgtgtgtgtgtgtgtgtgtgtgcgcgcacgtgcgcgcgtgtgcctttgtgttttccaggacagcctatctcaaacaaacaaacaaaaagtgataaagatgctaagtgtggctcttcacagttggtggcttctggcaggaatgtggatgggggaagactcagttctcttaaggggctggccactgagagtctgacgatgctccagtgagtacatgggtaacataaattgaacttggtagatgtggaaggaacacaaggggagaaaggtgttctggaggcgtgggaagcgagaggggtaggaatacattttataaaattcccaaataattaataaaaatattttgttggggaaaaagaagcagcagaggttggccaggttgacaaggctggcataagattgcagggcctcagagacaggaggtgggagcttggcagaggtgcaggggagagatctgggtcttagtaactgttctcttgctgtgaagagacaccatgaccacgcagcttatagaagaaagcatgagttgaggagttgcttacggtatcagagggtgaggccatgaccatcatggcagggagcttggtagcagcaggcatggttgctggagaagtaggtgaatctttacattctggtctgtaggcagcagacagacagagaaagaaacaaacctggcctggtgcaggctcttgaaacctcaaagctcactgccacaacacacctccttcgacaaggccacacctctcaatccttcccaaccagttcactgcttcacacacatgagcctgtggggccattggcattcaaaccatgacagtctggctggctgagtggggcaagccggccctgctgactcctgggcagggccttcaactcctcattccagaaggctctcatcgctgtcagctagagagccacgggcggtgttctcagggtgcagaaagttggacccatctctgctggtgaccttctttgccccccccccctctctctctctctctctctctgcctgccgttctcccttggtgccagtttctcagagtaatgctctctccctgcaccctggtcccaggccagttgaggagcttctatgtggaggccccaggagaggctgttgtcctggagctctcccctggcagtgtctacgtgtgactagcagggctcctcagagtctgccagtccagcccctagtgtcttccatctgtgatctcagagggctgagggacaggactcttggcatgatggagccaaagctaaggcctttggtcccacttaaagaagcagtaatgatctgggcccgctctgcctactccccacctaatgctctgggtctgtaaaatggagagttttacaggcctcagtacagcaaagcctagaagggtctgcagatgggatcacctgggcctacagcagcccctgcttccagttctcccatggtaggccgcctctgggcagcctaggcctctgccagtcatgctttgagagtcactataatgctagaccatagctccctgtgtctaagataagagtcttccagccccagtgtaccctgactttaggaaaggaggggcccagactcctaaggtggctcacacccactgtcagatgacttggatcctaggggcaccccttccctgctgtgtgagcttggttaagttaatcaacccctctgagcctacaaatcagatgcatagcaacatctcttctggaaggatttgaggggcagtgggtgtgcgtagggctgagcccggcctgccactcatggcactaattgtgcccggatactgtaccacttcccagttgctcctggagctcactccaaggagaacctgctgtgctcctgtgccttgtttgtagtatacgcctgcacagggctttctacaacacaggcaccgtggtgttctctttcaaatggttaatgttctgttgtgtggattctacatcagtaaattattaagaaaaagcaaaaacaaacaaatggcaggaggggtctccattgccctagtaccaggccacctcatggaaattaaagtttgtgtttgtagcgctggaggttgaagccaaggcctcctgaatacgtggaaggcacctgacatcgagctatagccttggcccctttcctttcctctttcacttgttcacactcgcatcagtggggtgtgtctgtcgtggcgcgggtagaggccagaaaacagcctcgcttgctgtgtgtacaccaggcttcctgcccctagggcttttgaaggtcctctgtcccattggaggcatgtggggttccaggtgctgctgttggtgggtctgcatgggttcttcacacctccatggcaagcactgtatctactaactcaccgctagctctagacccagccctttttccatctgtgtgtgtgtgtgtgtgtgtgtgcgtgtgtgtgtgtgtgtgtgtgtgtgtgtgtgtgtgtgtgtacaaccttgggtgtcgttctcagggatcatacacacacctttcttgttttcagatagggtccctcaatgccttggaacttgacctctgggcaaccagccccatgtatcaattcctgcctccattgatccggggctgggattacaaatccgtaatgccatgctcagctctgttgttgttgttgctgctgctgctgcgaataaggatactggaaatcaaactcaggcctgcgtgttctatggcaagcatgttaggactgacccatctctcgatcctcagaaatcccagcactcgggaggcagaggcaggcagatttctgagttggaggccagcctggtctacaaagtgagtttcaggacagtcaaggctatacagagaaaccctgtctcgaaaaaaaccaaaaaaaaaaaaaaaaaaaaagaagaagaagaagaagtagtcagggtaaagtaggtgggtcggcgggtaaaagtgcttgtggttggggctggagacatggctgagtggtccagttcccagcacctacacggccactcacaaacattgtaactccaattccagggcatccaacgcccttttcaggcctctgtgggcatcaggaatgcatgtgaggcacgggcatacattcaggcaaagcacctacacacgttcttcccaaaagtgcttgccacggaggcttgatgagctgctgcccacaagggaggagagattaattaattttttaaaaaaagccaacaacgctgggcgtggtggcgcacgtctttaatctcagcacttgggaggcagaggcaggtgaatttctgagttcgaggccagcctggtctacaaagtgagttccaggacagccagagctacacagagaaaccctgtctcgaaaaaaaaaaccaaaaaaacaaaaaaaaaaaaaaaaaaaaaaaaaaaaagccaacaacaattcctggatttgtgaattgaatatttctagtaaagcgatagtgaactgggagtagtggctcacgcccgtcatcctagtgcttatgaggtagagcagaaagatcagaagtacaagaccatcctcagctacatagcaggttcaaggccagtctagatgcatgagaccgtatttctaaaggctaaaatgctttttaaaggtaatattagaaacagaataggatagggcagagtttcatgcctggttccatgtctgagtggtggcaggtcaacactccagggtcactgccacctggatcaacactacgaggcgcacacacagggtctcctgcccctatgggagatgggcggatacatggagagactaaggtccaaaaaatgggaagatttggcagttggcctgaccccctcacctgcgtgtctgttctcacaggttggcctgcttgggcggctgccctgcctgacccacagccatctgtcagtccatctgtccgccttgaccccaggagacccagagatcgaggaaaggatcggagcatggtgtagacaccctcagccagcccagggagcccggccccgcacatctgagggaaggaagtggctggccaggtgagtggtctcgagacctctactcctctattcggtttggggacaatggcaggcagtggggacaggtgcatctcagggagcagggagagcctctctggggaagtggcatccaggtagaagccagaaggtgctctctgccacatggtggtcagcagtcagagtgaccggtgcaggcagagacccagcatctgtgggcgcctgatggagatagaacccaggactgtgaacacgccagaccatgtgctgacctctgacctctgtctcagcttctatttgttaccctttttatgtgagataggactttgcttttatagctctggcaatctgcctgcctctacttcctgactgctgggcttaaagccttgtacccacacctctggtttttgttttttttgttttaatgtgtgcattctttgtatgtgtgggtcacaacctgggtcagcaccgtgatccctttgtattgagtggatatcaggatgagataccatatggatccgcactgtctgtctgtctgtctgttcaatcctctggtagctgtcaccttctatctctggtgagcacgctactgggatgttccagtagtgtgtgaggccgcgctggagggatgctttgtgggttctttgcgcatagagtatcttgtctttgtttcatgggaggaagcctgagtgacggtgattctgggctggcactgtctctctgtgtgtctgtctgtctgtctgtctctctctctctgtctctctctctctcgctgaggcaagccaaaactatcacaggtcaagctgggggcacccacagtctgctgagggggtggatagtcgggacaaagctgggggaggggagctcatgtggctggaggccacatggaggagctcagccttaaagaataggaagtcaggctggagagatggctcagcggttaagagcactgactgttcttccagaggtcctgagttcaattcccagcaactacatggtggctcacaaccatctgtaatgggatctgatgctgtcttctgctgtgtctgaagacagtgacagaagggtgaaggtggagagagctaggggccggagcaatggctcagccgttaagagctcttgttcctacagaggaccttagtgtcagtccagcatgcccgtggtggcaagcagctttctgtgacttcagttcagggatctgttgccctcttctaccctccataggcaccaggcatacctactgtgcactaataatgttcatgtgcagtcaccaggcatgcacacagagtacatacacacatgcagacgagctggacatagcctccgtgtggtggcccaggcaccacccactggcactttgctggtcattgactcgtgagcttcccagagctgcagggagagaaacagacagcacatgcctgggagacatcagaagggagtggggctcagtggggttcagtggctcgagcagggaaaggtggccttctctttacaggactgtctgtccagatccaccgaaatggcccaccgccttgctcttcccagcctcccacttctagatctttgtcttaacacgcctcacagcatgatcaacagtagacaagacagcctgtgtgcccatccatcaggaatggagtcttggtgtgttccatttagtgaggcctttcctgtcaaaggcaaagacgccagtctgtgtgtcctcaagggaaagacgcccagaccagaccagagaacctcagctgttttaaaaaacctgtgctgtttccaggagagagcggccgccctgagggatgctcaatgtctgctttgccacactcagtctttttgtttagtgactgggatatggctgatttgggggagcgcttaccttgcaggcatgaagccctatgcttgatttccagcaccacattcacagctgtaatcccagaggcaggagaatgaggaattcaaggtcccccttagatacagcaggttcaaggccagcctgagacggaaggagaaaatgagaaatcccaggctagtggtagtttctgttatctttctcgttccttcgcttctttctaacagatgtagcccaggctggtccagactgtcctcaaactcacaatcttctgtccttggtcttccaagtactgggatcagaggcttgtacacagaggcctgttatgttgctaggctagccttgaaccccaccccacctccggaaccaaacagccctccctccctcagcctcctgagcagctgggatcacaggtgcacaccactagaagtggcttccgcttaactccaaggagggtctaggcacacttatgggacagaggacctgcatggtgggtttctcttccctctgggaattagactgagctaccacttcctgtgcataactctaggcctgcttccccaggaaatgcacctgccctgcagggttgatcgctggtcacagcagctgatgcctgcccagcaacaccaagagcactttattggcagtagtgtgtctggttgtcccttgagcctctcccccagcaccaaggaagggccctttgtctgtgcccaacaccctggccactgacttgctccgaccacacccacttctttctgctcctttggtggttcggttctcaccgaagcagagagaccgaccaccaagggactaaggcaaaagtcaaggtcttcctcaccccggcgagtctgagatgtaaatcaaagtcagaaatagaccccaagatcctcccccctcaccccaccccccccacccccccaaccccccagcccccccacccccacccaggtgtggtctagccacatctctctggagctgggcctgagaccacacggggcaggctggtgccggtgccggtgccaagtggccttccctagtgccaaggtctccatcccctcaggtcctgcggcccctctggatgcccgtgctggcctctcctctagcatcgtgcccaccatggcctcagctgacaagaatggcagcaacctcccatctgtgtctggtagccgcctgcagagccggaagccacccaacctctccatcaccatcccgccaccagagagccaggcccccggcgagcaggatagcatgcttcctgaggtgaggggctgcccgccacaggccacagatgtgactgcccacacagcaactagacacactcttccatctcagtctactgacagtcctgcagcctcaacatacgcctgaaagattgcagggagcaaggctctgaactctgaacttgggagttctgttgcctggtgaccaagggacaaggaacactgtccctgagaaaaggccttgttgggaagcctgggctctggctgtttacagttcccctccctggccctgctgtgggctgtctgtggaggcctgagtgggcatggggtcccacagtgccagcagccacccctacagcgccttgttcttggggggatgggtcattgtttacgtccatctgggactcttgccccataaagagctcattgaaaacaggccccaaatagcgtgggctttgaacacagcctgaaacataaaggggagctatgttggggccacccgctcaccctgagagtactctaagacacgtgagaagaggcaggacatcctggtatatgggcatatcatgggtagggagtgtttgcactgaggtttgtacagtggaaaacttgtgttttgtttgttgagacagggtctcgtgtagcccaggttagcctcgaacttgctctatagtcaacaagtactttgaactgctggtccttcttgctctacttcctgggtagaaaggtgctgagtggcaagtgttcaccgccgcaccggggggcggtgtgtagcgctgggacaggaacccagggctttaggcgtgctgggcacactgccaacctgagctgtatccccagaccttgctagaaaatgctcactttgggttcagaatgagggcctatggaggactgagtgcagccagcaggctagcgtgttaaagggtctgggctatgtcctcaactctgcaataaaaagggctgaatgggagcccaaaagcagccaacaggtgagtggcctagacactcattcatgtcctccttgcctccccacagaggcgcaagaacccagcctacctgaagagtgtcagcctacaggagccccggggacgatggcaggagggcgcagagaagcgccccggcttccgccgccaggcctccctgtcccagagcatccgcaagtgagcacctgagccctgcctggtcaccccagcggcctggcctttcccggggcctgagccctgtgtccccctttccagaggtgtagacaatcagggagtagcatctccccgtggatgtggaaggccagaatctgaagtgatggtgttaatgggatgagaaggggctggggctgggcctgtgcacctgacaggaagttccacagcacgctgcagaggggctctccagccttcccagtcccacccatcatggggtagccttcactctcagcctggtcgctgggctgtagctgggaactcaggggggtgtagggaagactcactgattccctagggccttgaagatacatggcagggtccatggtccattggttaccctgtatacacacaggaacacacatgccttaccctcaagtcccctctccccacacacaccgccatcccctagaccaccttccataaacacgagcaccatggcctcccgtcacctcatacccgtgtgcctcgtgtacccgcaactcacacatcaccctcccaacacacacacatgtaccgccatccccctgcctcctcctttaacacatgtaccttcgccctcacctcctcgtgcacacgcgtgcaccaggacaggtgcgtgcacaggtttctgcccacaccgtatctgttctgcaggagcacagcccagtggtttggggtcagcggcgactgggagggcaagcgacaaaactggcatcgtcgcagcctgcaccactgcagcgtgcactatggccgcctcaaggcctcgtgccagagagaactggagctgcccagccaggaggtgccatccttccagggcactgagtctccaaaaccgtgcaagatgcccaaggtgggccccctggaggtgatgggcagcaagcggctctcccagggtctgggcaacattgttcacccacatctcttgcagattgtggatccactggctcggggtagggccttccgccatccagatgaggtggaccggcctcacgctgcccacccacctctgactccaggggtcctgtctctcacatccttcaccagtgtccgctctggctactcccatctgccccgccgcaagaggatatctgttgcccatatgagctttcaggcagccgccgccctcctcaaggtaaggtctgcattgaaggatgtatgtccccgtccagggtagccactccactcacctacatgtctaccctctgtgtcagagtagtgaatgcatgcacaccctaaaggccagaacttatgtccttcaggcccacagttcccctgtgagccctcagcgcctcctctgctatgggttatgggagaagtggtgggggggggggaggtattggatgtagatgccttgagtgccgggtgccaactgttcccgttagcaggcaagagctcgtgagggaagagctgagagctgatcctgattactggaaggagaggctgtgtagggccggctgcagccagagccccatcctggcctgctctgagtacttccttcagcccgtctacttatccaatgtcctgtgcctttgccaacctggtcctggggttctgagccctccttgacgcttctgttccatgcccaggggcgttccgtgctagatgcgactgggcagcggtgccggcatgtcaaacgcagcttcgcttaccccagcttcctggaggaggatgctgtcgatggagctgacaccttcgactcctccttttttagtaaggcaagcatggggtgtggactctgggcgggggatggggtggcctgagagctaggaggagtgaccttggccttgactgtggcttgtgggatcccaggaaggtccagaggcagggtggggtatggcctttgtgagccattggtctgggctcccttagaagggggtgtggagtggagcacgaacccgctgggaaagttaggagtgacaagcatgcggctgcccttccatcctgaagagactgtatagctttgccttgcccttgattggagtgctacgatggtccttggggcagtggctcctcacactgtccttggtgtccctctactccaggaagaaatgagctccatgcctgacgatgtctttgagtcccccccactctctgccagctacttccgaggtgtcccacactctgcctccccggtctccccggatggagtgcacatcccgctgtgagtacagggtgggactctccagcccctgctgagccctggcagggtcctcaagctaagagcctctgtcatcaccagaaaagaatacagcggtggccgagccctgggtcccgggacccagcgtggcaaacgcattgcctccaaagtaaagcactttgcatttgaccggaagaagaggcactacggcctgggcgtcgtgggtaactggctcaaccgaagctatcgacgcagcatcagcagcaccgtgcagcggcagctggagagcttcgatagccaccggtgagcccccaggagccagtctgagcagaggactaagtggctagagtttccaaacctctcaggcctcttcatcccaaacatggccccttctcagtcagtaagcattctgagagcgcctcctgcaggtcctgcaggtaatgacaagctcctgggcacacacatgctcagggagcgctctgtagcctggagagagccagctgacactgctaccttatcctcaacatggcccccagacagcagtcagtcagtcagtcagtcagtcagtcaacaaacacagatcaaggacaactatagcagtctctggacacagcccagagagaggggcattgcgcctggacaggtgccctggggctggcaaggcggttcagtgaggctagggatgagtttgatccctggaaccaataaaaagaaaggagagaagcgactccacagagttgtcgcctgacgtccacatgcctgcgagtcatgttggggacacacacacacacaactctaataattttaaaatgatcttttaagcccagcatgctggcacatgcttttagttccagtattcaggaggcagaggcagacagaggtctatgagttcaaagctagcccggtctaaatagtgactactagctaatgctccatagagaaacgttctctcaaagccaaaaggcggttaaaagagaacaaagggagttggcgcctggagtgaggggtcctggggggagtggacacagagatgttcagggaaactggagattaaagagtgatagccaaagtgtggttgacagtgtgacctgataggcctcggactccaaattacacaaagtaagaaagggcaggagtcgaagtaagaaagggcaggagtggtggagtgggtggggagcgggtgggggacttttgggatagcattggaaatgtaaatgaaataaatacctaataaataaagtaaagaaaaagaaagggcaggagtggtgggcaagcggctagggtggaggggtgctgttagggtgggatgccgtgagcagaggtgtgggcccccccaggtgatgtgaagtgtgcacaaaggtcctggggcttcaggggccgtggcctggtccaggaagcacaagcagaggaggtgagcagcaattgcaggtgagcacaggcaggcttgccagttatcagcaggggatggctttgatcttcacacagtaggcagtgggaagccatcgtgggctttgaactgagagagacactggggatgcaccttccaagtttctgtggggaaggctgagctgggggaatgcctgccatgcccaggtgaagggcgtgaggagcggggcatgagctctgccaggctgactggaagcctcccccggcaggccctacttcacctactggctgacgttcgttcacatcatcatcaccttgctggtgatctgcacctatggcatcgcacctgtgggctttgcccagcacgttaccacccagctggtgagtagggttcctcctggggggtccccggcctctcccaaggagctttggcacagttggcaccaagtatctcccaccacagtaccctggcccaagttggagatgcctgaggctcatagcctctttctagaggcccttttctggggatgcccccacccccgtccctttctcttctcacgccgagggctctggctcctctaatgccacaaactaccttcttgataggtgctgaagaacagaggcgtgtatgagagcgtgaagtacatccagcaggagaacttctggattggccccagctcggtgaggcccaggatgcccgggagccctgtatcctgccactccacactgggcagaggggtggatggtagggtgccccgcccactgcttccgagtatggggcactggctcacagtcccccccccccccactccagattgacctcattcacctgggagcaaagttctcgccctgcatccggaaggaccagcaaattgagcagctggtacggagggagcgcgacattgagcgcacctctggctgctgtgtccagaatgaccgctcgggctgcatccagaccctgaagaaggactgctcggtgggtcctgcccctacccctgcacctgcccctgcccctgccagcctccttccttccccggcccaaagctgggcttggaaagctggacagtagttcctaggaagtacccaagtcctggggaaatgggaaaagcccttgtcctgcggggttcgctgcccacaccatctgactgacacggcctgaccacctgtccaccctctaggagactttagccacgttcgtaaagtggcagaatgatactgggccctcagacaagtctgacctgagccagaagcagccatcggcggttgtgtgccaccaagaccccaggtacagccaagagcctcctatgggtccagagtggcacccagctgttggagctgccatctgtgatgctggtgggtgggtgtggcaggctgggacatcccagccttttattttgctcaatctgacccctgcttctaggacctgtgaagagcctgcctccagtggggcccacatctggcctgatgacattaccaagtggccggtgagtaggagatccatggagaaaggtctgggatgagggtggggacagctggctttccgtcatgaggcccactgccattggtcccctgtctcttagatctgcacagagcaggctcagagcaaccacacgggcttgttgcacatagactgtaagatcaaaggccgcccctgctgcatcggcaccaagggcaggtgagccggtgcctccaaccaacccctgcaggctgatggacctctgtgactgtcttctgttctctgctcttgggtgatggggggaggcagggattgaggggcagtgatatagaaaggagcttgtcccatcctgcctgccatgcccagggactgggctccccagctgatgctctttagaacgctgacagccgtctgcttcctgtccacagctgcgagatcaccactcgggagtactgtgagttcatgcatggctatttccatgaagacgcgacgctgtgttcccaggtagtggaagctgtagggattctgagggccactggggtccttaccctttggaagcctcagacttggcttgcctcccagggcagaactactgaccctgtgtctcccacccaggtgcactgtttagacaaggtgtgtgggctcctgcctttcctcaaccctgaggtccctgaccagttctaccggatctggctgtctttattcctgcatgctgggtaagaggcaccctgttgccccatgctcagactcccatgtctcccctcttgggtgccggagaaaagggcttccagaccgagcacactggctcaacctgctagtgctagactgcgctgtggtgtgctctgcgggtgaccatgggcacacaggaaaggctgatggtgctcatgtgcctaccccagcatagtgcactgccttgtgtctgtggtcttccaaatgaccatcctgagggacctagagaagctggccggctggcaccgcatctccatcatcttcatccttagtggcattacaggcaacctggccagcgccatcttcctcccctaccgggcagaggtacaaacttgggagacaagggcagagagggtgggatgagcccttccttttggatctaaagctttataacatatggggaggaccattgtagcctgtggggaggaccattgtggcttgtcaggaggactattgtggcctgtggggaggatcactgtggcctatgggaaggaccaaacctgctgcttcttgctctggttccaccccagaatctgcaacaagggcagagtccctgttgtgtcaagcttacctatagatgggcagctaaggtagaacctattgattagcctcttaattcatgacaggagggaacagagatactcttgagtccccagagatccttgctccttgttctgtgaaaccctacatttggctcctctccaccctcaggaggagaggtcttgagtctgttgctccttctggcctgcgatctctccactgcctgtagtctcctaggacaggctggcttgtgctaagcacggggttagagcacacccaggtttgctgcagggttggacagagcagggccagcagctcctgtggcatcctccgagtgggagatgtgccccacagctggtacctggcacccagcatcagtggcgacatctctcctccctgaccccaggtgggcccagccgggtcgcagttcggcctcctcgcctgcctcttcgtggagctgttccagagctggcagctgttggagcggccgtggaaggccttcttcaacctgtcggccattgtgcttttcctcttcatctgtggcctcctgccctggatagacaacatcgcccacatcttcgggttcctcagcggcatgcttctggccttcgccttcctgccttacattaccttcggcaccagcgacaagtaccgcaagcgagccctcatcctcgtgtcgctgctggtctttgctgggctctttgcttccctggtgctgtggctgtacatctaccccatcaactggccctggatcgagtacctcacctgctttcccttcaccagccgcttctgtgagaagtacgagctagaccaggtgctacactaactgcagagattgtgtgtctgccctgggccgtgtgtctatgaaccggtggggccctgagcccagcagctcggtccacactcaaggctgactccagatgagacgggcggtaaaggcaggctccccagggagatgactcctcctttctcaggtctgaatgttcctgacccaagtctgggggacatccaggacacttgcttctctgaggctcaggtcccaggccctgcctgcctctctggctctataaagatgataacttttcttgggcctctggcctctgcgggtgctgtctccccactgacactgtgactgtgacctcgccagacacacagctgctctctcagttgtccccagggttgaggtccttaccatgctgctatgaccccgttttcctgtttctcctctcctccctgtccttcctgtgtttgtccgtggactcgtgagcctgctcctgaggctcctggacatagagcattgtggggaaggctctggctgttgtctatgggggatgacaagcaaggagagatggctatacagggatgctaggggcttttgttaagcaaagaagccagctctcctaagcccatcagctgccctagctccagcatgtgtctggctgcacaggttggctctgatcccaggatgcccctgccacctgccctcactcctgcgtggccgtgggccgagccgccttgaggactagctcccagaggaggcctgaggcccagactggtgggttttttggttttgttttgtttttttttttccaatttatattatggtcctaattttgtaaagtaacgctaactttgtacggatgatgtctcaagtttattaaatgacattctttattaaaatgctgccttttgccttagacctccaagagaaggaaagctagaacttggggctacagagatggctcacttcctatagagaacctgggttcagttcagagatccgcctgcctctgcctcccaagtactgggattaaaggtgtgcgctaccaccgccccagccaaaacaatgtttttatttgaaagagaaagccccggggccttgaggctgaagcagaccggaacacttgggatctctgtcatttagttgaactagaaaccagattgagatctcagcagagcccccaaggaccctgagagaactgggttacgtgtagagccctgagacctcaaccccagctgctctgcctttgctccagggacatcaggggctgatgggcagcaggagtggcctgttccagcagaggtggaccagcggggaggaggccacgtgtttgctccagtcagctcgaagaagaccctacacacacccatgaggcacagccactggggacatagctactttattcgtgggcagaggtgccagtcctgtggctggtgggggaaccagggaggcaggggggtgggccggcacattggtggctgactgcagtttggtgtggc

SEQ ID NO: 3 - protein, 856 aa, Mus musculus
MSEARRDSTSSLQRKKPPWLKLDIPAAVPPAAEEPSFLQPLRRQAFLRSVSMPAETARVPSPHHEPRRLVLQRQTSITQTIRRGTADWFGVSKDSDSTQKWQRKSIRHCSQRYGKLKPQVIRELDLPSQDNVSLTSTETPPPLYVGPCQLGMQKIIDPLARGRAFRMADDTADGLSAPHTPVTPGAASLCSFSSSRSGFNRLPRRRKRESVAKMSFRAAAALVKGRSIRDGTLRRGQRRSFTPASFLEEDMVDFPDELDTSFFAREGVLHEEMSTYPDEVFESPSEAALKDWEKAPDQADLTGGALDRSELERSHLMLPLERGWRKQKEGGPLAPQPKVRLRQEVVSAAGPRRGQRIAVPVRKLFAREKRPYGLGMVGRLTNRTYRKRIDSYVKRQIEDMDDHRPFFTYWLTFVHSLVTILAVCIYGIAPVGFSQHETVDSVLRKRGVYENVKYVQQENFWIGPSSEALIHLGAKFSPCMRQDPQVHSFILAAREREKHSACCVRNDRSGCVQTSKEECSSTLAVWVKWPVHPSAPDLAGNKRQFGSVCHQDPRVCDEPSSEDPHEWPEDITKWPICTKSSAGNHTNHPHMDCVITGRPCCIGTKGRCEITSREYCDFMRGYFHEEATLCSQVHCMDDVCGLLPFLNPEVPDQFYRLWLSLFLHAGILHCLVSVCFQMTVLRDLEKLAGWHRIAIIYLLSGITGNLASAIFLPYRAEVGPAGSQFGILACLFVELFQSWQILARPWRAFFKLLAVVLFLFAFGLLPWIDNFAHISGFVSGLFLSFAFLPYISFGKFDLYRKRCQIIIFQVVFLGLLAGLVVLFYFYPVRCEWCEFLTCIPFTDKFCEKYELDAQLH

SEQ ID NO: 4 - DNA, 199 bp, Artificial Sequence
  donor oligonucleotide for introduction of a mutation in wild-type Rhbdf2 gene
cgtgcaagatgcccaaggtgggccccctggaggtgatgggcagcaagcggctctcccagggtctgggcaacattgttcacccacatctcttgcagattgtggatctactggctcggggtagggccttccgccatccagatgaggtggaccggcctcacgctgcccacccacctctgactccaggggtcctgtctctcac

SEQ ID NO: 5 - protein, 7 aa, Artificial Sequence
  nuclear localization signal from SV40 large T-antigen
PKKKRKV

SEQ ID NO: 6 - protein, 14 aa, Artificial Sequence
  nuclear locaization signal from nucleoplasmin
RPAATKKAGQAKKK

SEQ ID NO: 7 - DNA, 17 bp, Artificial Sequence
  sequence encoding sgRNA for introduction of a mutation in the mouse Rhbdf2 gene
gcagattgtggatccac

SEQ ID NO: 8 - DNA, 20 bp, Artificial Sequence
  PCR primer for identifying a point mutation in the mouse Rhbdf2 gene
acacacacatgtaccgccat

SEQ ID NO: 9 - DNA, 20 bp, Artificial Sequence
  PCR primer for identifying a point mutation in the mouse Rhbdf2 gene
ttctggcctttagggtgtgc